(19) United States
(12) Patent Application Publication
(10) Pub. No.: US 2016/0144021 A1
(43) Pub. Date: May 26, 2016

(54) Vaccine Composition And Method Of Use

(71) Applicant: MEDIMMUNE, LLC, Gaithersburg, MD (US)

(72) Inventors: Stacie Lynn Lambert, Redwood City, CA (US); Elizabeth Ann Stillman, San Jose, CA (US); Roderick Tang, San Mateo, CA (US); Jennifer Chui Ling Woo, Los Altos, CA (US); Gary Van Nest, Seattle, WA (US)

(21) Appl. No.: 14/782,840

(22) PCT Filed: Apr. 4, 2014
(86) PCT No.: PCT/US14/32938

§ 371 Date: Oct. 7, 2015

Related U.S. Application Data
(60) Provisional application No. 61/809,563, filed on Apr. 8, 2013.

(51) Int. Cl. A61K 39/39; A61K 39/155; C12N 7/00; A61K 9/107

(52) U.S. Cl. CPC A61K 39/39; A61K 9/107; A61K 39/155; C12N 7/00; A61K 2039/55572

(57) ABSTRACT

Described herein is a vaccine composition and methods of use. In one embodiment, the vaccine composition includes RSV-F protein in combination with an adjuvant. In a more particular embodiment, the vaccine composition includes RSV soluble F protein in combination with a lipid toll-like receptor (TLR) agonist. In a more particular embodiment, the adjuvant comprises Glucopyraonsyl Lipid A (GLA). In a further embodiment, the adjuvant comprises GLA in a stable oil-in-water emulsion (GLA-SE).

### CLAIM OF PRIORITY

This application claims the benefit of prior U.S. Provisional Application No. 61/809,563, filed on Apr. 8, 2013, which is incorporated by reference in its entirety.

### REFERENCE TO SEQUENCE LISTING SUBMITTED ELECTRONICALLY

The content of the electronically submitted sequence listing in ASCII text file (Name: RSVFseqlist.txt; Size: 46,202 bytes; and Date of Creation: Apr. 3, 2014) filed with the application is incorporated herein by reference in its entirety.

### FIELD OF THE INVENTION

The invention relates generally to vaccines which provide protection or elicit protective antibodies to viral infection. More specifically, vaccine preparations against Respiratory Syncytial Virus (RSV), and more particularly, human Respiratory Syncytial Virus Fusion protein (RSV-F) are described.

### BACKGROUND

Respiratory syncytial virus (RSV) is the leading cause of serious lower respiratory tract disease in infants and children (Feigen et al., eds., 1987, In: Textbook of Pediatric Infectious Diseases, WB Saunders, Philadelphia at pages 1653-1675; New Vaccine Development, Establishing Priorities, Vol. 1, 1985, National Academy Press, Washington D.C. at pages 397-409; and Ruuskanen et al., 1993, Curr. Probl. Pediatr. 23:50-79). The yearly epidemic nature of RSV infection is evident worldwide, but the incidence and severity of RSV disease in a given season varies by region (Hall, C. B., 1993, Contemp. Pediatr. 10:92-110). In temperate regions of the northern hemisphere, it usually begins in late fall and ends in late spring. Primary RSV infection occurs most often in children from 6 weeks to 2 years of age and uncommonly in the first 4 weeks of life during nosocomial epidemics (Hall et al., 1979, New Engl. J. Med. 300:393-396). Children at increased risk from RSV infection include preterm infants (Hall et al., 1979, New Engl. J. Med. 300:393-396) and children with bronchopulmonary dysplasia (Groothuis et al., 1988, Pediatrics 82:199-203), congenital heart disease (MacDonald et al., New Engl. J. Med. 307:397-400), congenital or acquired immunodeficiency (Ogra et al., 1988, Pediatr. Infect. Dis. J. 7:246-249; and Pohl et al., 1992, J. Infect. Dis. 165:166-169), and cystic fibrosis (Abman et al., 1988, J. Pediatr. 113:826-830). The fatality rate in infants with heart or lung disease who are hospitalized with RSV infection is 3%-4% (Navas et al., 1992, J. Pediatr. 121:348-354).

RSV infects adults as well as infants and children. In healthy adults, RSV causes predominantly upper respiratory tract disease. It has recently become evident that some adults, especially the elderly, have symptomatic RSV infections more frequently than had been previously reported (Evans, A. S., eds., 1989, Viral Infections of Humans. Epidemiology and Control, 3^{rd} ed., Plenum Medical Book, New York at pages 525-544). Several epidemics also have been reported among nursing home patients and institutionalized young adults (Falsey, A. R., 1991, Infect. Control Hosp. Epidemiol. 12:602-608; and Garvie et al., 1980, Br. Med. J. 281:1253-1254). Finally, RSV may cause serious disease in immunosuppressed persons, particularly bone marrow transplant patients (Hertz et al., 1989, Medicine 68:269-281).

Treatment options for established RSV disease are limited. Severe RSV disease of the lower respiratory tract often requires considerable supportive care, including administration of humidified oxygen and respiratory assistance (Fields et al., eds, 1990, Fields Virology, 2^{nd} ed., Vol. 1, Raven Press, New York at pages 1045-1072). The antiviral agent ribavirin has been approved for treatment of infection (American Academy of Pediatrics Committee on Infectious Diseases, 1993, Pediatrics 92:501-504). It has been shown to be effective in the treatment of RSV pneumonia and bronchiolitis, modifying the course of severe RSV disease in immunocompetent children (Smith et al., 1991, New Engl. J. Med. 325:24-29). However, ribavirin has had limited use because it requires prolonged aerosol administration and because of concerns about its potential risk to pregnant women who may be exposed to the drug during its administration in hospital settings.

One major obstacle to vaccine development is safety. A formalin-inactivated vaccine, though immunogenic, unexpectedly caused a higher and more severe incidence of lower respiratory tract disease due to RSV in immunized infants than in infants immunized with a similarly prepared trivalent parainfluenza vaccine (Kim et al., 1969, Am. J. Epidemiol. 89:422-434; and Kapikian et al., 1969, Am. J. Epidemiol. 89:405-421). As such, despite over 50 years of research, no suitable vaccines against RSV have been developed. Thus, there remains a compelling unmet medical need for a safe and efficacious vaccine against RSV.

### SUMMARY OF THE INVENTION

A vaccine composition is described herein. In particular, the vaccine composition includes RSV-F protein. In one embodiment, the vaccine composition includes RSV soluble F protein. In one embodiment, the RSV soluble F protein lacks a C-terminal transmembrane domain. In a more particular embodiment, the RSV soluble F protein lacks a cytoplasmic tail domain. In one embodiment, the RSV soluble F protein comprises amino acids 1-524 of RSV soluble F protein from human strain A2 (SEQ ID NO: 2). In another embodiment, the RSV soluble F protein comprises SEQ ID NO. 7.

In a more particular embodiment, the vaccine composition includes RSV soluble F protein in combination with an adjuvant. In one embodiment, the adjuvant is a lipid toll-like receptor (TLR) agonist. In one embodiment, the adjuvant is a (TLR)4 agonist. In one embodiment, the adjuvant is a synthetic hexylated Lipid A derivative. In a more particular embodiment, the adjuvant includes Glucopyraonsyl Lipid A (GLA). In one embodiment, the adjuvant includes a compound having a formula:

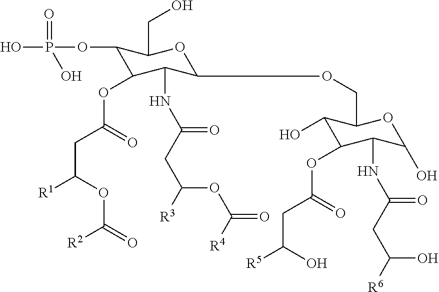

wherein R1, R3, R5 and R6, are C11-C20 alkyl; and R2 and R4 are C12-C20 alkyl. In one embodiment, the adjuvant includes GLA in a stable oil-in-water emulsion (GLA-SE). In another embodiment, the adjuvant includes GLA in a stabilized squalene based emulsion.

In one embodiment, at least about 1 μg and up to about 200 μg RSV-F protein is included in the vaccine composition. In one embodiment, RSV-F protein includes soluble RSV-F protein. In one embodiment, at least about 1 μg and up to about 20 μg adjuvant is included in the vaccine composition. In one embodiment, the adjuvant includes GLA. In a more particular embodiment, the adjuvant includes GLA-SE. In a more particular embodiment, the adjuvant includes GLA in a stabilized oil-in-water emulsion having a concentration of at least about 1% and up to about 5%. In one embodiment, the adjuvant includes GLA in a stabilized oil-in-water emulsion having a mean particle size of at least about 50 nm and up to about 200 nm. In one embodiment, the vaccine composition also includes a pharmaceutically acceptable carrier, diluent, excipient, or combination thereof. The vaccine composition can be formulated for parenteral administration, for example intramuscular or subcutaneous administration. In one embodiment, the vaccine composition has a volume of between about 50 μl and about 500 μl.

In another embodiment, a method of preventing respiratory syncytial virus (RSV) infection in a mammal is provided. In one embodiment, the method includes administering to the mammal a therapeutically effective amount of a vaccine composition as described herein. In another embodiment, a method of inducing an immune response in a mammal, wherein the method includes administering to the mammal, an effective amount of a vaccine composition described herein. In another embodiment, a method for enhancing a Th1 biased cellular immune response in a mammal that has been previously exposed to RSV, wherein the method includes administering to the mammal an effective amount of a vaccine composition described herein. In one embodiment, the cellular immune response of the mammal includes a Th1 cellular immune response and a Th2 cellular immune response at a ratio of at least about 1.2:1. In another embodiment, a method of inducing neutralizing antibodies against RSV in a mammal, wherein the method includes administering to the mammal an effective amount of a vaccine composition described herein. In one embodiment, the RSV neutralizing antibody titers are greater than 10.0 Log 2. In one embodiment, RSV neutralizing antibody titers after administration of the vaccine composition include serum IgG titers that are at least about 4 fold compared to serum IgG titers before administration. In one embodiment, RSV neutralizing antibody titers after administration of the vaccine composition include serum IgG titers that are at least about 10 fold and up to about 200 fold greater compared to serum IgG titers before administration. In one embodiment, a method of reducing RSV viral titers in a mammal, wherein the method includes administering to the mammal an effective amount of a vaccine composition described above. In one embodiment, RSV viral titers following infection are reduced between about 50 and about 1000 fold. In another embodiment, RSV viral titers are less than 2 log 10 pfu/gram after administration of the vaccine composition. In a more particular embodiment, the RSV viral titers are less than 2 log 10 pfu/gram between about 1 week and 1 year after administration of the vaccine composition.

In one embodiment, the mammal is a human. In another embodiment, the mammal is an elderly human. In a more particular embodiment, the mammal is an elderly human that has attained a chronological age of at least about 50 years old. In one embodiment, the mammal is RSV seropositive.

In one embodiment, the vaccine composition is administered in a single dose regimen. In another embodiment, the vaccine composition is administered in a two dose regimen that includes a first and a second dose. In one embodiment, the second dose is administered at least about 1 week, 2 weeks, 3 weeks, 1 month or 1 year after the first dose. In another embodiment, the vaccine composition is administered in a three dose regimen.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIGS. 1A-F are graphs showing immune responses to adjuvanted RSV sF vaccines in naïve BALB/c mice. Mice (N=7 per group) were immunized at days 0 and 14 with the indicated vaccines and challenged with 6 log₁₀ PFU of RSV at day 28. Representative data shown from 1 of 2 experiments run with all groups. (A) Lung Viral Titers. Residual virus in the lungs of animals 4 days post challenge was quantified by plaque assay. Individual results are presented in log₁₀, along with a bar representing the group geometric mean. Individuals with undetectable titers were scored at the assay limit of detection (LOD), ˜1.4 log₁₀. (B) Serum RSV-GFP Neutralizing Titers. Individual Day 28 sera results are presented as the log₂ dilution of serum that provides 50% reduced fluorescent focus units (FFU) of virus, with a bar representing the group geometric mean. Individuals with undetectable titers were scored at the assay limit of detection (LOD) of 3.3 log₂, indicated by a dashed line. Significant differences (by 1 way ANOVA) are indicated by ***. (C) F-specific CD4 T-cell Cytokine Responses. Splenocytes (n=3 per group) were harvested 4 days post challenge and restimulated 72 hours. Shown are the specific IFNγ, IL-5, IL-13, and IL-17 responses to an immunodominant MHC II restricted RSV-F peptide pool calculated by subtracting media control values from test values in multiplexed cytokine analysis. The group means and SEM are shown. (D) Serum F-specific IgG1 and IgG2a Titers. Day 28 sera (n=7 per group) were evaluated for F-specific IgG1 and IgG2a isotypes by endpoint titer ELISA. Data is presented as the log₂ reciprocal serum endpoint dilution with a limit of detection (LOD) of 5.64 log₂. Shown is the group geometric mean with 95% confidence interval, with significant differences between groups (by 1 way ANOVA) indicated by ***. (E) IFNγ ELISPOT. Splenocytes harvested at 4 days post challenge were restimulated with an immunodominant RSV-F-derived MHC I restricted peptide to evaluate CD8 T cell responses. Significant differences between groups (by 1 way ANOVA) are indicated by ***. Individual mouse results are shown, along with a bar representing the group mean, for 3 animals/group in a representative experiment (repeated 2-7 times). (F) Granzyme B ELISPOT. Splenocytes were harvested and treated as for the IFNγ ELISPOT. Significant differences between groups (by 1 way ANOVA) are indicated by ***. Individual mouse results are shown, along with a bar representing the group mean, for 3 animals/group.

FIGS. 2A and B are graphs showing antigen dose titration effects on IFNγ for a composition including RSV-sF with fixed and varying amounts of GLA-SE. (A) Antigen dose titration effects on IFNγ ELISPOT. Individual mouse results are shown, along with a bar representing the group mean, for 5 animals/group given indicated doses of RSV sF in a fixed amount of GLA-SE. (B) Adjuvant dose titration effects on IFNγ ELISPOT. Individual mouse results are shown, along with a bar representing the group mean, for 3-4 animals/group given the indicated doses of GLA-SE with a fixed 0.3 μg amount of RSV sF.

FIGS. 3A-D are graphs showing F-specific CD4 and CD8 T-cell Induction and Priming. Mice were immunized intramuscularly at days 0 and 14 with 10 μg of RSV sF alone or formulated with the indicated GLA-SE or SE adjuvants. Splenocytes (n=5 per group) were harvested and restimulated with the indicated F peptides in an IFNγ ELISPOT at the indicated timepoints, either at day 28 (14 days post boost), or at day 32, 4 days following a challenge with 6 log₁₀ RSV A2. Individual animal results for one of two representative experiments are shown along with group means, with significant differences between groups (by 1 way ANOVA) indicated by ***. (A) 14 day post boost CD4 responses. IFNγ ELISPOT responses to a MHC II-restricted (CD4) F peptide pool at 14 days post boost. (B) 14 day post boost CD8 responses. IFNγ ELISPOT responses to an immunodominant MHC I-restricted (CD8) F peptide at 14 days post boost. (C) 4 day post challenge CD4 responses. IFNγ ELISPOT responses to a MHC II-restricted (CD4) F peptide pool at 4 days post challenge. (D) 4 day post challenge CD8 responses. IFNγ ELISPOT responses to an immunodominant MHC I-restricted (CD8) F peptide at 4 days post challenge.

FIGS. 4A and B are graphs showing recall CD8 T-cell responses to RSV in the Lung. Mice were immunized with the indicated vaccine formulations at days 0 and 14 (using 0.3 μg of RSV sF per immunization), then challenged with 6 log₁₀ pfu of RSV at day 28. Lungs were harvested 4, 7, or 12 days post challenge (n=3 for each group and timepoint) and restimulated 6 hours with either (A) an RSV-F-derived H-2K^{d} restricted peptide or (B) an RSV M2-derived H-2K^{d} restricted peptide. Cells were surface stained for CD3 and CD8, intracellularly stained for IFNγ, TNFα, and IL-2, and analyzed on an LSR2 for the frequency of responding CD8 T cells. The group mean is shown with significant differences between groups (by 1 way ANOVA) indicated by ***. Representative data from 1 of 2 experiments is presented.

FIGS. 5A-F are graphs and histology specimens showing lung responses to RSV challenge in naïve BALB/c mice. Mice (n=7 per group) were immunized at days 0 and 14 with the indicated vaccines and challenged with 6 log₁₀ pfu of RSV at day 28. Lungs were harvested 4 days post challenge. Representative data shown from 1 of 2 experiments run with all groups. (A-F) Cytokines in Lung Homogenates. Levels of IL-5, IL-13, IFNγ, IL-17, and eotaxin in clarified lung homogenates were quantified by multiplexed cytokine analysis and calculated as the amount per gram of lung harvested. Individual mouse results are shown, along with a bar representing the group mean. To calculate the IFNγ to IL-5 ratio, values were first zero-adjusted by adding 1 to each value before calculating.

FIG. 6A-F Pulmonary Cellular Infiltration. Formalin-fixed lung sections were H&E stained and evaluated for inflammatory markers. Shown are representative 10× field views for each group.

FIGS. 7A-F are graphs showing immune responses to adjuvanted RSV sF vaccines in naïve cotton rats. Animals were immunized at day 0 and day 21 with the indicated vaccine formulations (using 0.3 μg of RSV sF per immunization) and challenged at day 42 with 6 log₁₀ pfu of RSV. (A) Lung Viral Titers. Lungs were harvested at 4 days post challenge from individual animals (n=8 per group) with residual virus quantified by plaque forming assay. Individual results are shown in log₁₀, along with a bar representing the group mean. The dotted line indicates a 3 log₁₀ diminishment in residual virus compared to the control PBS+GLA-SE group. Significant differences (by 1 way ANOVA) between individual groups and the Live RSV A2 group are indicated by ***. (B) Nose Viral Titers. Noses and nasal turbinates were harvested at 4 days post challenge from individual animals (N=8 per group) with residual virus quantified by plaque forming assay. Individual results are shown in log₁₀, along with a bar representing the group mean. The dotted line indicates a 3 log₁₀ diminishment in residual virus compared to the control PBS+GLA-SE group. Significant differences (by 1 way ANOVA) between individual groups and the Live RSV A2 group are indicated by ***. (C) Serum RSV Neut Titers. Day 42 sera (N=5 per group) were heat inactivated and tested for neutralization of RSV-GFP infection of target cells by fluorescent focus assay. Data is presented as the log₂ dilution of serum that provides 50% reduced fluorescent focus units (FFU) of virus with a limit of detection (LOD) of 3.3 log₂ indicated by a dashed line. Individual results are shown, along with a bar representing the group mean and 95% confidence interval. Significant differences (by 1 way ANOVA) between individual vaccine groups are indicated by ***. (D) Serum IgG Titers specific for RSV sF. Day 42 sera (N=5 per group) were tested for binding of RSV sF by endpoint ELISA. Data is presented as the log₂ dilution of serum that generates an OD>3× background with a limit of detection (LOD) of 3.3 log₂ indicated by a dashed line. Individual results are shown, along with a gray bar representing the group mean and 95% confidence interval. Significant differences (by 1 way ANOVA) between individual vaccine groups are indicated by ***. (E) IFNγ ELISPOT. Splenocytes (N=4-5 per group) harvested 4 days post challenge were restimulated with either media or with RSV sF protein in an IFNγ ELISPOT. F-specific responses were quantified by subtracting the media control values from the test values. Significant differences (by 1 way ANOVA) between individual vaccine groups are indicated by ***. (F) Ratio of IFNγ to IL-4 ELISPOT responses. F-specific IL-4 ELISPOT responses were evaluated and the ratio of IFNγ to IL-4 spot forming units was calculated following a zero-adjustment of values by adding 1 to each value.

FIGS. 8A-F are histologic samples showing lung responses to RSV challenge in cotton rats. Cotton Rats were immunized at days 0 and 21 with the indicated vaccines and challenged with 6 log₁₀ pfu of RSV at day 42. Lungs were harvested 4 days post challenge. Formalin-fixed lung sections were H&E stained and evaluated for inflammatory markers. Shown are representative 10× field views from each group (n=5 per group).

FIGS. 9A and B are photographs of a gel analysis of affinity-purified RSV sF protein. Purified sF protein was resolved in a 10-12% polyacrylamide gel under reducing (lane a) and non-reducing (lane b) conditions and visualized with Sypro Ruby. Molecular mass markers are shown in the margins.

FIGS. 10A and B are graphs showing mouse serum anti-sF antibody titers. Animals were immunized at day 0 and day 14 with the indicated doses of RSV sF without adjuvant or with GLA-SE and challenged at day 28 with 6 log₁₀ pfu of RSV. (A) Day 28 sera were evaluated for F-specific IgG by endpoint titer ELISA. Log₂ reciprocal serum dilutions for individual animals are shown with a bar representing the group geometric mean. The assay limit of detection (LOD) was 5.64 log₂ indicated by the dotted line. (B) Day 32 sera were evaluated for F-specific IgA by endpoint titer ELISA. Log₂ reciprocal serum dilutions for individual animals are shown with a gray bar representing the group geometric mean. The assay limit of detection (LOD) was 4.32 log₂ indicated by the dotted line.

FIGS. 11A and B are graphs showing the determination of an optimal in vivo dose of RSV sF antigen in naïve BALB/c mice. Animals were immunized at days 0 and 14 with the indicated doses of RSV sF (0.01-1.5 μg) without adjuvant or with 5 μg GLA-SE and challenged at day 28 with 6 log₁₀ pfu of RSV. (A) Plaque assay for residual viral titers in the lung measured in log₁₀ pfu/gram, 4 days post challenge. (B) RSV serum neutralizing titers in log₂ reciprocal serum dilutions, day 28.

FIG. 12 is a graph showing intracellular cytokine staining. Mice (n=3-5 per group) were immunized at days 0 and 14 with the indicated vaccines and challenged with 6 log₁₀ pfu of RSV at day 28. Splenocytes were harvested at Day 32, 4 days post challenge and restimulated with an immunodominant RSV-F-derived MHC I restricted peptide to evaluate CD8 T cell responses. Quantitation of polyfunctional IFNγ, TNFα, IL-2+CD8+ T cells by intracellular cytokine staining and flow cytometric analysis.

FIG. 13 is a table showing cross-neutralization of multiple RSV isolates by immune sera from naïve BALB/c mice immunized at day 0 and day 14 with PBS or with RSV sF+GLA-SE. Day 28 sera was tested for neutralization of RSV clinical isolates from a wide US geographical distribution (NY, CO, CA, NM/AZ) obtained over the last 10 years.

FIGS. 14A and B are graphs showing serum F-specific IgG endpoint titers for post vaccination timepoints in BALB/c mice made seropositive by a single infection with RSV 28 days prior to vaccination with the indicated RSV sF doses (0.4, 2, or 10 μg) without or with GLA-SE (5 μg in 2%). Sera were evaluated at each indicated timepoint for F-specific IgG by endpoint titer ELISA with a cutoff value of A₄₅₀>3×mean background. Data is presented in log₂ with a limit of detection (LOD) of 5-5.64. (A) Individual animal results are shown to illustrate seropositivity at Day 0, along with a bar representing the group mean, n=8-9 per group. (B) The group mean F-specific IgG titer at each time point post vaccination for n=6-9 animals is shown, with error bars depicting the 98% confidence intervals.

FIG. 15 is a graph showing a time course of serum RSV neutralizing titers following vaccination of 1× seropositive BALB/c mice. Sera from individual mice at each timepoint were heat inactivated and tested by fluorescent focus assay for neutralization of RSV-GFP infection of target cells in the absence of complement. Data is presented as the log₂ dilution of serum that reduced fluorescent focus units (FFU) by 50%. Values <the limit of detection (LOD) of 3.32 are reported as 2.32 for calculation purposes. The group geometric mean at each time point for n=6-9 animals is shown, with error bars depicting the 95% confidence intervals. Groups with p<0.05 by one-way ANOVA versus the PBS (seronegative) group are marked by *.

FIG. 16 is a graph showing serum F-specific IgA at day 14 following vaccination of 1× seropositive BALB/c mice. Serum endpoint antibody titers in animals 14 days post vaccination were quantified by ELISA using 3 fold serial dilutions, N=5-6 per group. Data is presented in log 2, with an LOD of 4.32 for the assay. Individual mouse results are shown, along with group means and error bars representing 95% confidence interval. Significant differences (p<0.05) compared to seropositive group vaccinated with PBS are indicated by *.

FIGS. 17A and B are graphs showing serum F-specific IgG1 and IgG2a titers at Day 0 and Day 42 following vaccination of 1× seropositive BALB/c mice. Sera were evaluated for F-specific IgG1 and IgG2a isotypes by endpoint titer ELISA with a cutoff value of A₄₅₀>3× mean of the blank. Data is presented in log₂. Bars represent the group geometric mean with 95% confidence interval. (A) N=8-9 animals/group with a limit of detection (LOD) of 4.05 for IgG1 and 4.5 for IgG2a. (B) N=5-6 animals with a LOD of 5.0 for both assays.

FIGS. 18A-C are graphs showing serum site specific competition ELISA at day 42 following vaccination of 1× seropositive BALB/c mice. Sera from individual animals 42 days post vaccination were evaluated over a dilution range of 1:25 to 1:2×10⁶ for RSV-F site-specific antibodies by competition ELISA with site-specific mAb 1121, 8599, and 1331H that bind to site A, B and C respectively, N=6 per group. In this assay, lower detected absorbances are indicative of greater competition by the polyclonal serum to binding of site-specific mAb to RSV sF. The percent competition (100×[1-{seraOD/mAbODmean}]) at a representative dilution of 1:125 is shown for individual mouse sera with bars representing the group mean. Significance (p<0.05) compared to the paired unadjuvanted group is indicated by **.

FIGS. 19A and B are graphs showing CD4 T-cell cytokine responses to RSV sF in vaccinated 1× seropositive BALB/c mice at Day 10 and Day 73 following vaccination. Splenocytes were harvested and restimulated either with media or with RSV sF protein to evaluate CD4 T cell responses, N=3 per group. IFNγ, IL-10, IL-5, and IL-17 in supernatants following 72-hour restimulation was measured by Bioplex multiplexed cytokine analysis. F-specific responses were calculated by subtracting the media control values from the test values. The group means with error bars representing the standard deviations are shown. A) Day 10 post vaccination, n=3 per group. B) Day 73, 4 days post RSV challenge, n=3-5 per group.

FIGS. 20A and B are graphs showing CD8 T-cell response to an immunodominant RSV-F peptide in 1× seropositive BALB/c mice at Day 10 following vaccination. Splenocytes were harvested 10 days post vaccination and restimulated with an immunodominant RSV-F-derived MHC I restricted peptide to evaluate CD8 T-cell responses, N=3 per group. (A) IFN-γ ELISPOT. Individual results are shown, along with a bar representing the group mean. (B) Polyfunctional IFNγ, TNFα, IL-2+CD8+ T cells as a percent of total CD8+ T cells following 6 hr restimulation measured by flow cytometric analysis of intracellular cytokine staining. The group means and standard errors are shown.

FIGS. 21A and B are graphs showing CD8 T-cell responses to an immunodominant RSV-F peptide in 1× seropositive BALB/c mice at Day 73 following vaccination. Splenocytes were harvested 4 days post challenge and restimulated with an immunodominant RSV-F-derived MHC I restricted peptides to evaluate CD8 T cell responses, N=3-5 per group. (A) IFN-γ ELISPOT. Individual results are shown, along with a bar representing the group mean. (B) Polyfunctional IFNγ, TNFα, IL2+ CD8+ T cells in selected groups as a percent of total CD8+ T cells following 6-hour restimulation measured by flow cytometric analysis of intracellular cytokine staining. The group means and standard errors are shown.

FIG. 22 is a graph showing cytokine responses in lung homogenates harvested from 1× seropositive BALB/c mice vaccinated prior to re-challenge with RSV. Cytokines in the lungs of animals 4 days post challenge with 6 log₁₀ pfu of RSV were quantified by multiplexed cytokine analysis of lung homogenates, N=5-6 per group. Individual mouse results are shown for the two most important cytokines (IFNγ and IL-5), along with a bar representing the group mean and 95% confidence interval.

FIG. 23 is a graph showing serum F-specific IgG by ELISA in 1× seropositive BALB/c mice prior to vaccination with sF vaccines. Data was quantified by ELISA in comparison to a reference standard and is presented in mg/mL equivalents. Individual animal results are shown, along with a bar representing the group mean, n=8-9 per group.

FIG. 24 a graph showing serum F-specific IgG1 and IgG2a isotypes by ELISA 2 weeks following vaccination of 1× seropositive BALB/c mice with RSV sF vaccines. Data was quantified by ELISA in comparison to a reference standard and is presented in mg/mL equivalents, with a limit of detection (LOD) of 8 mg/mL. Bars represent the group geometric mean with 95% confidence interval of N=6-7 animals/group.

FIG. 25 is a graph showing a timecourse of serum RSV neutralizing titers following vaccination of 1× seropositive BALB/c mice. Sera from individual mice at each timepoint were heat inactivated and tested by fluorescent focus assay for neutralization of RSV-GFP infection of target cells in the absence of complement. Data is presented as the log 2 dilution of serum that reduced fluorescent focus units (FFU) by 50%. Values <the limit of detection (LOD) of 3.32 are reported as 2.32 for calculation purposes. The group geometric mean at each time point for n=6-9 animals is shown, with error bars depicting the 95% confidence intervals.

FIGS. 26A and B are graphs showing CD8 T-cell responses to an immunodominant RSV F peptide in 1× seropositive BALB/c mice at 10 days following vaccination. Splenocytes were harvested 10 days post vaccination and restimulated with an immunodominant RSV F-derived MHC I restricted peptide, N=3-4 animals per group. A) IFN-γ ELISPOT. Individual results are shown, along with a bar representing the group mean. B) Polyfunctional IFNγ, TNFα, IL-2+CD8+ T cells as a percent of total CD8+ T cells following 6 hr restimulation measured by flow cytometric analysis of intracellular cytokine staining. The group means and standard errors are shown.

FIG. 27 is a graph showing a time course of serum RSV neutralizing titers following vaccination of 1× seropositive BALB/c mice with 10 μg RSV sF without or with various adjuvants. Sera from individual mice at each timepoint were heat inactivated and tested by fluorescent focus assay for neutralization of RSV-GFP infection of target cells in the absence of complement. Data is presented as the log₂ dilution of serum that reduced fluorescent focus units (FFU) by 50%. Values <the limit of detection (LOD) of 3.32 are reported as 2.32 for calculation purposes. The group geometric mean at each time point for n=6-9 animals is shown, with error bars depicting the 95% confidence intervals. Groups with p<0.05 by one-way ANOVA versus the PBS (seronegative) group are indicated.

FIGS. 28A-B are graphs showing lung responses to RSV challenge in naïve BALB/c mice. Mice (n=7 per group) were immunized at days 0 and 14 with the indicated vaccines and challenged with 6 log₁₀ pfu of RSV at day 28. Lungs were harvested 4 days post challenge. Representative data shown from 1 of 2 experiments run with all groups. (A-B) Cytokines in Lung Homogenates. Levels of eotaxin and IL-13 in clarified lung homogenates were quantified by multiplexed cytokine analysis and calculated as the amount per gram of lung harvested. Individual mouse results are shown, along with a bar representing the group mean.

FIGS. 29A and B are graphs showing CD8 T-cell response to an immunodominant RSV-F peptide in 1× seropositive BALB/c mice at Day 10 following vaccination with 10 μg RSV sF without or with various adjuvants. Splenocytes were harvested 10 days post vaccination and restimulated with an immunodominant RSV-F-derived MHC I restricted peptide to evaluate CD8 T-cell responses, N=3 per group. (A) IFN-γ ELISPOT. Individual results are shown, along with a bar representing the group mean. (B) Polyfunctional IFNγ, TNFα, IL-2+CD8+ T cells as a percent of total CD8+ T cells following 6 hr restimulation measured by flow cytometric analysis of intracellular cytokine staining. The group means and standard errors are shown.

FIGS. 30A and B are graphs showing RSV replication kinetics in unvaccinated naïve Sprague Dawley rats. Naive 5-6 week old female Sprague Dawley rats received 2×10⁶ pfu of RSV A2 at Day 0 by intranasal inoculation. RSV titers were quantified by plaque assay using serial dilutions of clarified lung or nose homogenates, N=5 per time point. Individual results along with a bar representing the group geometric mean RSV titer are shown. The highest limit of detection (LOD) is indicated by the solid line. Individuals with titers <LOD were given a value of 4.0 for graphing and statistical calculations.

FIGS. 31A-D are graphs showing serum cytokines, 6 hours post vaccination of naïve Sprague Dawley rats. Sera were evaluated for rat IL-6, MCP-1, MIP-1β and GRO/KC by multiplexed bead-based ELISA. Quantitation in μg/mL was determined by comparison to standard curves. Bars represent the group geometric mean with 95% confidence interval, N=5-6 animals/group. The lower limit of detection is indicated by a dashed line. Individuals with titers <LOD were given a value equal to the LOD for graphing and statistical calculation.

FIGS. 32A-C are graphs showing RSV sF specific serum IgG titers following vaccination of naïve Sprague Dawley rats. Endpoint titers of RSV-F-specific IgG by ELISA are presented in log₂. Bars represent the group geometric mean with 95% confidence interval from n=3 animals/group at Day 14 and n=4-6 animals/group at Days 22 and 42. Samples below the assay limit of detection (LOD) of 5.64 (indicated by a dashed line) were given a value of 5.64 for graphing. * indicates p<0.05 vs PBS group and ** indicates p<0.05 versus matched RSV sF group using 1-way ANOVA with Tukey's post test. A) Day 14 post vaccination. B) Day 22 post vaccination C) Day 42 post vaccination.

FIG. 33 is a graph showing serum RSV sF-specific isotypes, Day 42 following vaccination of naïve Sprague Dawley rats. RSV sF-specific IgG1, IgG2a, and IgG2b isotypes were measured by endpoint ELISA. Bars represent the group geometric mean from n=4-6 animals/group. Samples less than the assay limit of detection (LOD) of 5.64 (indicated by a dashed line) were assigned a value of 5.64 to allow for graphing. * indicates p<0.05 versus PBS group and ** indicates p<0.05 versus matched RSV sF group by 1-way ANOVA with Tukey's post test.

FIGS. 34A and B are graphs showing RSV neutralizing titers following vaccination of naïve Sprague Dawley rats. Sera from individual rats vaccinated with the indicated vaccines and controls were heat inactivated and tested by fluorescent focus assay for their ability to neutralize RSV-GFP infection of target cells in the absence of complement. Data is presented as the log₂ dilution of serum that reduced fluorescent focus units (FFU) by 50%. Samples less than the limit of detection (LOD) of 3.32 were given a value of 3.30 for graphing and statistical calculations. Individual results are shown for n=4-6 animals per group with the group geometric mean displayed along with error bars depicting the 95% confidence intervals. * indicates p<0.05 versus PBS group and ** indicates p<0.05 versus matched unadjuvanted sF group by 1-way ANOVA with Tukey's post test.

FIG. 35 is a graph showing RSV F-specific IFNγ ELISPOT response in splenocytes from vaccinated naïve Sprague Dawley rats. Splenocytes were harvested 4 days post RSV A2 challenge and evaluated by IFNγ ELISPOT for responses to RSV sF protein restimulation, N=4-6 per group. Individual results as well as the group mean and standard deviations are displayed for each treatment group. * indicates p<0.05 compared to PBS and ** indicates p<0.05 compared to paired unadjuvanted sF by 1-way ANOVA using Bonferroni's multiple comparison post test.

FIGS. 36A and B are graphs showing RSV A2 titers post challenge in vaccinated naïve Sprague Dawley rats. At Day 42, all vaccine groups were challenged IN with 2×10⁶ pfu of RSV A2. RSV titers were quantified by plaque assay at 4 days post challenge using serial dilutions of clarified lung or nose homogenates, N=4-6 per group. Individual results along with a bar representing the group geometric mean RSV titer are shown. The limit of detection (LOD) is indicated by the solid line (8.7 pfu/gram for lungs, 4.0 pfu/gram for nose), and samples below the LOD were assigned the LOD value for graphing and statistical calculations.

FIGS. 37A and B are graphs showing the weight change over time in naïve rodents given RSV sF vaccines. (A) Naïve cotton rats were vaccinated at Day 0 and Day 21 with 0.3 μg RSV sF without or with adjuvants GLA-SE (5 μg/2%), GLA (5 rig), SE (2%), or alum (100 μg) and tracked for their percent weight change from day 0 through day 25. (B) Naïve Sprague Dawley rats were vaccinated at Day 0 and Day 21 with 10-100 μg RSV sF without or with GLA-SE (2.5 μg/2%) and tracked for their percent weight change from day 0 through day 42.

FIG. 38 is a graph showing the neutralization titers in RSV seropositive mice. Mice were dosed with 1×106 PFU RSVA2 via an intranasal route on day 0 and day 35. Neutralizing Ab titers on Day 28 (following 1 dose of live RSVA2) and Day 56 (28 days post second dose of live RSVA2) were quantified by microneutralization assay with a lower LOD of 3.3. Titers of naive mouse subset are also shown. Titers were calculated as log 2 of the closest dilution that resulted in a 50% reduction in FFU. If the first serum dilution (1:10) did not provide the fluorescent focus unit count <=50% of the input virus, the titer was reported as 10 and a value of 3.3 log 2 was use for analysis. N=8 for naive mice and N=35 for Day 28 and Day 56. Mean with SD is shown.

FIG. 39 is a graph showing the neutralizing antibody responses 14 days post immunization. Neutralizing Ab titers at 14 days post immunization (Day 70) were quantified by microneutralization assay with a lower LOD of 3.3. Titers were calculated as log 2 of the closest dilution that resulted in a 50% reduction in FFU. If the first serum dilution (1:10) did not provide the fluorescent focus unit count <=50% of the input virus, the titer was reported as 10 and a value of 3.3 log 2 was imputed for analysis. N=4 mice with mean and SD shown.

FIG. 40 is a graph showing Neutralizing Antibody Responses over the Duration of the Study. Neutralizing Ab titers at Day 56, Day 70 and Day 84 were quantified by microneutralization assay with a lower LOD of 3.3. Titers were calculated as log 2 of the closest dilution that resulted in a 50% reduction in FFU. If the first serum dilution (1:20) did not provide the fluorescent focus unit count <=50% of the input virus, the titer was reported as 10 and a value of 3.3 log 2 was imputed for analysis. N=8 mice for Day 56 and N=4 mice for Day 70 and Day 84 with mean and SEM shown.

FIG. 41 is a graph showing Baseline RSV F specific IgG Responses in Seropositive Mice prior to vaccination. Total anti-F IgG serum titers were quantified by ELISA on RSV sF coated plates for individual mouse sera. The monoclonal antibody 1331H (Beeler and van Wyke Coelingh, 1989) was used to generate a standard curve. N=8 mice for naive group and N=35 mice with 2 serial infections of RSV. Mean and SD are shown.

FIG. 42 is a graph showing Total RSV F Specific IgG Titers 14 Days Post Immunization. Total anti-F IgG serum titers were quantified by ELISA on RSV sF coated plates for individual mouse sera and the log 2 of the titer is graphed. The purified monoclonal antibody 1331H (Beeler and van Wyke Coelingh, 1989) was used to generate a standard curve. N=4 with mean SD shown. Statistical analysis by 1-way ANOVA and Tukey post-test.

FIG. 43 is a graph showing Total RSV F Specific IgG Titers Over the Duration of the Study. Total anti-F IgG serum titers were quantified by ELISA on RSV sF coated plates for individual mouse sera and the log 2 of the titer is graphed. The purified monoclonal antibody 1331H (Beeler and van Wyke Coelingh, 1989) was used to generate a standard curve. N=4 with mean SD shown. N=4 with mean SD shown.

FIG. 44 is a graph showing RSV F-specific IgG1 and IgG2a Responses. Anti-F IgG1 or IgG2a serum levels at Day 84 (28 days post-immunization) were quantified by ELISA on RSV sF coated plates for individual mouse sera. The purified monoclonal antibody 1331H or 1308 (Beeler and van Wyke Coelingh, 1989) was used to generate a standard curve. N=4 with mean and SEM shown.

FIGS. 45A-B are graphs showing Lung Cytokine Titers 4 Days Post-RSV Challenge: IFNγ and IL-5Lung cytokine titers in supernatants from lung homogenates isolated at 4 days post challenge were measured by Bioplex multiplexed cytokine analysis. N=4 mice with SEM shown.

FIGS. 46A-C are graphs showing Lung Cytokine Titers 4 Days Post-RSV Challenge: Eotaxin (FIG. 46A), IL-13 (FIG. 46B) and RANTES (FIG. 46C). Lung cytokine titers in supernatants from lung homogenates isolated at 4 days post challenge were measured by Bioplex multiplexed cytokine analysis. N=4 mice with SEM shown.

FIGS. 47A-B are graphs showing CD8 T-Cell F-Peptide Splenocyte Restimulation by ELISPOT. Spleens were isolated either at 11 days post immunization (FIG. 47A) or 4 days post-challenge (FIG. 47B). Splenocytes were stimulated with an F-specific CD8 T-cell epitope and the number of IFNγ secreting cells was determined by ELISPOT assay. Group means of 4 mice are shown. Statistical analysis by 1-way ANOVA and Tukey post-test.

FIGS. 48A-D are graphs showing the Total RSV F IgG Serum Responses in seropositive cotton rats. Total anti-F IgG serum levels were compared by ELISA on RSV sF coated plates for individual mouse sera at a dilution of 1:1000. Group means for N=8 animals for Days 28 and 38 and group means for N=5 animals on Days 49 and 56 with SD is shown. The cotton rat positive control serum was pooled from cotton rats that received 4 repeated serial immunizations of 1×10⁶ PFU RSV A2 intranasally at 2 week intervals. The cotton rat negative control serum was pooled from naive animals.

FIGS. 49A-B are graphs showing Neutralizing Antibody Response in seropositive cotton rats. Neutralizing Ab titers at Day 28 and Day 49 were quantified by microneutralization assay with a lower LOD of 3.3. Titers are the log 2 of the EC50 calculation of the dilution that generates a 50% reduction in FFU. Group means for N=8 animals for Day 28 and N=5 animals for Day 49 and the SD are shown. If the first serum dilution (1:10) did not provide the fluorescent focus unit (FFU) count <50% of the input virus, the titer was reported as 10, and a value of 3.3 [log 2(10)] was imputed for analysis.

FIGS. 50A-C are graphs showing Fold Rises in Neutralizing Antibody Titers in seropositive cotton rats. Neutralizing Ab titers at Day 28, 38, 49 and 56 were quantified by microneutralization assay. EC50 values were calculated as the dilution that generates a 50% reduction in FFU of the input virus. Fold rises were calculated by dividing the EC50 value at the indicated day by the EC50 value at Day 28 for each cotton rat. Group geometric means for N=8 animals for Day 38 and N=5 animals for Day 49 and 56 with the 95% confidence intervals are shown. A value of one indicates no boost in neutralizing titers.

FIGS. 51A-C are graphs showing Site Specific Antibody Responses at Day 56 in seropositive cotton rats. Sera from individual animals at Day 56 were evaluated over a dilution range of 1:25 to 1:2×10⁶ for RSV F site-specific antibodies by competition ELISA with Synagis®, 1112, and 1331H that bind to site A, B and C respectively. The percent competition (100×[1−{seraOD/mAbODmean}]) at a representative dilution of 1:125 is shown for individual sera. The group mean for N=5 animals with SD is shown.

FIGS. 52A-B are graphs showing Total RSV F IgG Serum Responses in seropositive cotton rats. Total anti-F IgG serum levels were quantified by endpoint dilution ELISA on RSV-sF coated plates for individual mouse sera with a LOD of 6.6 log 2. Endpoints were calculated as the log 2 of the highest dilution that resulted in an OD greater than 2 times the mean of the blank. If the first serum dilution (1:100) was not higher than 2 times the mean of the blank the titer was reported as 100, and a value of 6.6 (log₂100) was use for analysis. Group means for N=11 animals with SD is shown. The cotton rat positive control serum was pooled from cotton rats that received 4 repeated serial immunizations of 1×10⁶ PFU RSV A2 intranasally at 2 week intervals. The cotton rat negative control serum was pooled from naive animals. Statistical analysis by 1-way ANOVA and Tukey post-test.

FIG. 53 is a graph showing Fold Rise in RSV F specific IgG Titers in seropositive cotton rats. RSV sF-specific IgG titers were quantified on Day 28 and 3. The fold rise was calculated by raising 2 to the power of the value obtained by subtracting the log 2 endpoint titer at Day 28 from the log 2 endpoint titer on Day 38 for each cotton rat. Group geometric means for N=11 animals with the 95% confidence intervals are shown. Dotted lines are at Y=1 and Y=4. A value of one indicates no boost in neutralizing titers. The cotton rat positive control serum was pooled from cotton rats that received 4 repeated serial immunizations of 1×10⁶ PFU RSV A2 intranasally at 2 week intervals. The cotton rat negative control serum was pooled from naive animals.

FIGS. 54A-B are graphs showing RSV Neutralizing Antibody Response in seropositive cotton rats. Neutralizing Ab titers at Day 28 and Day 38 were quantified by microneutralization assay with a lower LOD of 3.3. Titers are the log 2 of the EC50 calculation of the dilution that generates a 50% reduction in FFU. Group means for N=11 animals with SD are shown. If the first serum dilution (1:10) did not provide the fluorescent focus unit (FFU) count ≦50% of the input virus, the titer was reported as 10, and a value of 3.3 [log 2(10)] was imputed for analysis. The cotton rat positive control serum was pooled from cotton rats that received 4 repeated serial immunizations of 1×10⁶ PFU RSV A2 intranasally at 2 week intervals. The cotton rat negative control serum was pooled from naive animals. Statistical analysis by 1-way ANOVA and Tukey post-test.

FIG. 55 is a graph showing Fold Rises in RSV Neutralizing Antibody Titers in seropositive cotton rats. Neutralizing Ab titers at Day 28 and 38 were quantified by microneutralization assay. EC50 values were calculated as the dilution that generates a 50% reduction in FFU of the input virus. Fold rises were calculated by dividing the EC50 value at the indicated day by the EC50 value at Day 28 for each cotton rat. Group geometric means for N=11 animals with the 95% confidence intervals are shown. The dotted lines are at 1 and 4. A value of one indicates no boost in neutralizing titers.

FIGS. 56A-C are graphs showing Site Specific Antibody Responses at Day 38 in seropositive cotton rats. Sera from individual animals at Day 56 were evaluated over a dilution range of 1:25 to 1:2×10⁶ for RSV F site-specific antibodies by competition ELISA with Synagis®, 1112, and 1331H that bind to site A, B and C respectively. The percent competition (100×[1−{seraOD/mAbODmean}]) at a representative dilution of 1:125 is shown for individual sera. The group mean for N=11 animals with SD is shown. The cotton rat positive control serum was pooled from cotton rats that received 4 repeated serial immunizations with 1×106 PFU of RSV A2 intranasally at 2 week intervals. The cotton rat negative control serum was pooled from naive animals. Statistical analysis by 1-way ANOVA and Tukey post-test.

FIGS. 57A and B are graphs demonstrating the time course of anti-F IgG antibody titers in individual cynomolgus monkeys, from Day −7 through Day 183. Vaccines (either RSV sF for group 1 or RSV sF+GLA-SE for group 2) were administered at Days 0, 28, and 169 as indicated by the arrows. Anti-F IgG titers for individual animals are presented in log 2 values at tested time points, with an assay limit of detection of 6.6 log 2 (equivalent to a 1:100 serum dilution). Values below the limit of detection are estimated at 6.0 for visualization.

FIGS. 58A and B are graphs demonstrating the time course of RSV neutralizing antibody titers in individual cynomolgus monkeys, from Day −7 through Day 183. Vaccines (either RSV sF for group 1 or RSV sF+GLA-SE for group 2) were administered at Days 0, 28, and 169 as indicated by the red arrows. Neutralizing IC₅₀ titers for individual animals are presented in log 2 values at tested time points, with an assay limit of detection of 2.3 log 2 (equivalent to a 1:5 serum dilution). Values below the limit of detection are estimated at 2.3 for visualization.

FIGS. 59A and B are graphs demonstrating the timecourse of IFNgamma ELISPOT responses in individual cynomolgus monkeys, from Day −7 through Day 183. Vaccines (either RSV sF for group 1 or RSV sF+GLA-SE for group 2) were administered at Days 0, 28, and 169 as indicated by the red arrows. Individual results for each animal are presented in spot forming cells (SFC) per million PBMC at tested timepoints. Responders (animals displaying both a 4-fold rise and a >50 SFC/million change from baseline) are indicated by the asterixes.

### DETAILED DESCRIPTION

### 1. Definitions

Unless otherwise defined herein, scientific and technical terms shall have the meanings that are commonly understood by those of ordinary skill in the art. Further, unless otherwise required by context, singular terms shall include pluralities and plural terms shall include the singular.

The term “about” as used herein refers to the range of error expected for the respective value readily known to the skilled person in this technical field.

As used herein the term “adjuvant” refers to a compound that, when used in combination with a specific immunogen in a formulation, will augment or otherwise alter or modify the resultant immune response. Modification of the immune response can include intensification or broadening the specificity of either or both antibody and cellular immune responses. Modification of the immune response can also mean decreasing or suppressing certain antigen-specific immune responses.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(abs′)2, and Fu fragments), single chain Fu (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. The term “antibody” can also refer to a Y-shaped glycoprotein with a molecular weight of approximately 150 kDa that is made up of four polypeptide chains: two light (L) chains and two heavy (H) chains. There are five types of mammalian Ig heavy chain isotypes denoted by the Greek letters alpha (α), delta (δ), epsilon (ε), gamma (γ), and mu (μ). The type of heavy chain defines the class of antibody, i.e., IgA, IgD, IgE, IgG, and IgM, respectively. The γ and α classes are further divided into subclasses on the basis of differences in the constant domain sequence and function, e.g., IgG1, IgG2A, IgG2B, IgG3, IgG4, IgA1 and IgA2. In mammals there are two types of immunoglobulin light chains, X and K. The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

As use herein, the term “antigenic formulation” or “antigenic composition” refers to a preparation which, when administered to a vertebrate, especially a bird or a mammal, will induce an immune response.

As used herein, the stages of life include: youth, reproductive maturity, and elderly. The term “youth” refers to a mammal from newborn to the point at which the mammal has attained reproductive maturity. The term “reproductive maturity” refers to a mammal that is at an age where mammals of that species are generally capable of mating and reproducing. As used herein, the term “elderly” refers to a mammal from reproductive maturity to death. The term “elderly” can be defined in terms of chronology (i.e., age in years); change in social role (i.e. change in work patterns, adult status of children and menopause); and/or change in capabilities (i.e. invalid status, senility and change in physical characteristics). In terms of chronology, when referring to human mammals, the term “elderly” generally refers to a person that has attained the chronological age of at least about 50, 55, 60 or 65 years old.

As used herein, “viral fusion protein” or “fusion protein” or “F protein” refers to any viral fusion protein, including but not limited to, a native viral fusion protein or a soluble viral fusion protein, including recombinant viral fusion proteins, synthetically produced viral fusion proteins, and viral fusion proteins extracted from cells. As used herein, “native viral fusion protein” refers to a viral fusion protein encoded by a naturally occurring viral gene or viral RNA that is present in nature. The term “soluble fusion protein” or “soluble F protein” refers to a fusion protein that lacks a functional membrane association region, typically located in the C-terminal region of the native protein. As used herein, the term “recombinant viral fusion protein” refers to a viral fusion protein derived from an engineered nucleotide sequence and produced in an in vitro and/or in vivo expression system. Viral fusion proteins include related proteins from different viruses and viral strains including, but not limited to viral strains of human and non-human categorization. Viral fusion proteins include type I and type II viral fusion proteins. Numerous RSV-Fusion proteins have been described and are known to those of skill in the art.

As used herein, the terms “immunogens” or “antigens” refer to substances such as proteins, peptides, peptides, nucleic acids that are capable of eliciting an immune response. Both terms also encompass epitopes, and are used interchangeably.

As use herein, the term “immunogenic formulation” refers to a preparation which, when administered to a vertebrate, e.g. a mammal, will induce an immune response.

As used herein, “pharmaceutical composition” refers to a composition that includes a therapeutically effective amount of RSV-F protein together with a pharmaceutically acceptable carrier and, if desired, one or more diluents or excipients. As used herein, the term “pharmaceutically acceptable” means that it is approved by a regulatory agency of the Federal or a state government or listed in the U.S. Pharmacopia, European Pharmacopia or other generally recognized pharmacopia for use in mammals, and more particularly in humans.

As used herein, the term “pharmaceutically acceptable vaccine” refers to a formulation that contains an RSV-F immunogen in a form that is capable of being administered to a vertebrate and that induces a protective immune response sufficient to induce immunity to prevent and/or ameliorate an infection or disease, and/or to reduce at least one symptom of an infection or disease. In one embodiment, the vaccine prevents or reduces at least one symptom of RSV infection in a subject. Symptoms of RSV are well known in the art. They include rhinorrhea, sore throat, headache, hoarseness, cough, sputum, fever, rales, wheezing, and dyspnea. Thus, in one embodiment, the method can include prevention or reduction of at least one symptom associated with RSV infection. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of a RSV infection or additional symptoms, a reduced, severity of a RSV symptoms or a suitable assays (e.g. antibody titer and/or T-cell activation assay).

As used herein, the term “effective amount” refers to an amount of antigen necessary or sufficient to realize a desired biologic effect. The term “effective dose” generally refers to the amount of an antigen that can induce a protective immune response sufficient to induce immunity to prevent and/or ameliorate an infection or disease, and/or to reduce at least one symptom of an infection or disease. The term a “therapeutically effective amount” refers to an amount which provides a therapeutic effect for a given condition and administration regimen.

As used herein, the term “naïve” refers to a person or an immune system which has not been previously exposed to a particular antigen, for example, RSV. A naïve person or immune system does not have detectable antibodies or cellular responses against the antigen. The term “seropositive” refers to a mammal or immune system that has previously been exposed to a particular antigen and thus has a detectable serum antibody titer against the antigen of interest. The term “RSV seropositive” refers to a mammal or immune system that has previously been exposed to RSV antigen. A seropositive person or immune system can be identified by the presence of antibodies or other immune markers in serum, which indicate prior exposure to a particular antigen.

As used herein, the phrase “protective immune response” or “protective response” refers to an immune response mediated by antibodies against an infectious agent or disease, which is exhibited by a vertebrate (e.g., a human), that prevents or ameliorates an infection or reduces at least one disease symptom thereof. The RSV-F protein vaccines described herein can stimulate the production of antibodies that, for example, neutralize infectious agents, blocks infectious agents from entering cells, blocks replication of the infectious agents, and/or protect host cells from infection and destruction. The term can also refer to an immune response that is mediated by T-lymphocytes and/or other white blood cells against an infectious agent or disease, exhibited by a vertebrate (e.g., a human), that prevents or ameliorates infection or disease, or reduces at least one symptom thereof.

As use herein, the term “vertebrate” or “subject” or “patient” refers to any member of the subphylum cordata, including, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like are also non-limiting examples. The terms “mammals” and “animals” are included in this definition. Both adult and newborn individuals are intended to be covered. In particular, infants and young children are appropriate subjects or patients for a RSV vaccine.

As used herein, the term “vaccine” refers to a preparation of dead or weakened pathogens, or antigenic determinants derived from a pathogen, wherein the preparation is used to induce formation of antibodies or immunity against the pathogen. In addition, the term “vaccine” can also refer to a suspension or solution of an immunogen (e.g. RSV-F protein) that is administered to a vertebrate, for example, to produce protective immunity, i.e., immunity that prevents or reduces the severity of disease associated with infection.

### 2. Viral Fusion Glycoproteins

Viral fusion glycoproteins mediate entry of a virus into a host cell during viral infection via membrane fusion induction and include precursor (F₀) proteins, with or without a signal peptide, and activated and/or mature fragments, including F₁ and F₂ subunits. As used herein, the terms “mature” and “activated” refer to viral fusion proteins that have been converted from a precursor protein to the mature fusion protein by host proteases. Typically, activated viral fusion proteins include a membrane-anchored and a membrane-distal subunit, which are named F₁ and F₂, respectively. The active F₁ and F₂ subunits are often linked together via a disulfide bond.

### 3. Human Respiratory Syncytial Virus (RSV) Proteins

Human respiratory syncytial virus (RSV) is a member of the family Paramyxoviridae, subfamily Pneumovirinae and genus Pneumovirus. RSV is divided into two subgroups, A and B, which are differentiated primarily on the variability of the G gene and encoded protein. RSV is an enveloped virus characterized by a single stranded negative sense RNA genome encoding three transmembrane structural proteins (F, G and SH), two matrix proteins (M and M2), three nucleocaspid proteins (N, P and L) and two nonstructural proteins (NS1 and NS2).

The two major protective antigens of RSV are the envelope fusion (F) and attachment (G) glycoproteins that are expressed on the surface of Respiratory Syncytial Virus (RSV), and have been shown to be targets of neutralizing antibodies. These two proteins are also primarily responsible for viral recognition and entry into target cells. G protein binds to a specific cellular receptor and the F protein promotes fusion of the virus with the cell. The F protein is also expressed on the surface of infected cells and is responsible for subsequent fusion with other cells leading to syncytia formation. Thus, antibodies to the F protein can neutralize virus or block entry of the virus into the cell or prevent syncytia formation. Although antigenic and structural differences between A and B subtypes have been described for both the G and F proteins, the more significant antigenic differences reside on the G protein. Conversely, antibodies raised to the F protein show a high degree of cross-reactivity among subtype A and B viruses. Consequently, F protein is an attractive target for neutralizing RSV, because it is present on the viral surface and therefore accessible to immunosurveillance. Additionally, F protein is less variable compared to G protein.

The F protein is a type I transmembrane surface protein that has an N-terminal cleaved signal peptide and a membrane anchor near the C-terminus. In nature, the RSV-F protein is expressed as a single inactive 574 amino acid precursor designated F₀. In vivo, F₀ oligomerizes in the endoplasmic reticulum and is proteolytically processed by an endoprotease to yield a linked heterodimer containing two disulfide-linked subunits, F₁ and F₂. The smaller of these fragments is termed F₂ and originates from the N-terminal portion of the F₀ precursor. The N-terminus of the F₁ subunit that is created by cleavage contains a hydrophobic domain (the fusion peptide), which associates with the host cell membrane and promotes fusion of the membrane of the virus, or an infected cell, with the target cell membrane. In one embodiment, the F-protein is a trimer or multimer of F₁/F₂ heterodimers.

Suitable RSV-F proteins for use in the compositions described herein can be from any RSV strain or isolate known in the art, including, for example, Human strains such as A2, Long, ATCC VR-26, 19, 6265, E49, E65, B65, RSB89-6256, RSB89-5857, RSB89-6190, and RSB89-6614; or Bovine strains such as ATue51908, 375, and A2Gelfi; or Ovine strains.

In one embodiment, an RSV-F protein for use herein can include an amino acid sequence that is at least about 90%, 91%, 92%, 93%, 94%, 95%, 96%, 97%, 98% or 99% identical to an RSV-F amino acid sequence provided herein, or can include 1, 2, 3, 4, 5, 6, 7, 8, 9, 10, 11, 12, 13, 14, 15, 16, 17, 18, 19 or 20 amino acid modifications with respect to an RSV-F amino acid sequence provided herein. For example, the amino acid sequence of the wild-type RSV-F Human strain A2, for example, is set forth in SEQ ID NO: 2.

Native, full-length viral fusion proteins typically include a membrane association region. Recombinant soluble viral fusion proteins can be generated, which lack a functional membrane association region, which often is located in the C-terminal region of the native protein. Recombinant soluble viral fusion proteins can be generated by deletion, mutation, or any mode of disruption known in the art, of the functional membrane associated region of a viral fusion protein. For example, any part or all of the membrane association region can be removed or modified provided that the membrane association region is not detectably functional (e.g. region no longer reside in the membrane), and (ii) a certain percent of the membrane association region remains (e.g., about 50% or less remains), is removed (e.g., about 50% or more removed) or is modified (e.g., about 50% or more modified). The extent to which the disrupted membrane associated region no longer confers association of the protein to the plasma membrane can be determined by any technique known in the art that can assess membrane association of proteins. For example, co-immunostaining of the viral fusion protein and a known membrane associated protein can be performed to visualize protein retained in the membrane. Examples of soluble viral fusion proteins are provided herein and include soluble RSV-F protein. Soluble RSV-F protein is also is referred to herein as RSV-sF. Soluble RSV-F can be generated, for example, by deletion of at least 50%, 60%, 70%, 75%, 80%, 85%, 90%, 95%, or 100% of the 50 amino acid C-terminal transmembrane domain of the RSV-F protein, corresponding to amino acid 525-574 of SEQ ID NO: 2. The amino acid sequence for a soluble RSV-F is set forth in SEQ ID NO: 7.

Three nonoverlapping antigenic sites (A, B, and C) and one bridge site (AB) have been identified for the fusion glycoprotein of the A2 strain of respiratory syncytial virus (RSV-F A2). (Beeler and Wyke Coelingh, (1989) “Neutralization Epitopes of the F Glycoprotein of Respiratory Syncytial Virus: Effect of Mutation upon Fusion Function,” J. Virol. 63(7):2941-2950). In one embodiment, the RSV-F protein includes one or more intact A, B or C neutralizing epitopes. In one embodiment, the RSV-F protein includes at least the A epitope. In another embodiment, the RSV-F protein includes at least the B epitope. In another embodiment, the RSV-F protein includes at least the C epitope. In other embodiments, the RSV-F protein includes at least the A and B epitopes, at least the B and C epitopes, or at least the A and C epitopes. In another embodiment, the RSV-F protein includes all three neutralizing epitopes (i.e., A, B and C).

### 4. Recombinant Expression of RSV-F

In one embodiment, a vaccine composition includes RSV-F protein. As used herein, the term “RSV-F protein” refers to full-length wild-type RSV-F protein, as well as variants and fragments thereof, including, for example, RSV soluble F protein (also referred to as RSV-sF). In a one embodiment, the vaccine composition includes recombinantly produced RSV-F protein. In a more particular embodiment, the vaccine composition includes recombinantly produced soluble RSV-F protein.

To recombinantly produce an RSV-F protein, an open reading frame (ORF) encoding the viral fusion protein may be inserted or cloned into a vector for replication of the vector, transcription of a portion of the vector (e.g., transcription of the ORF) and/or expression of the protein in a cell. The term “open reading frame” (ORF) refers to a nucleic acid sequence that encodes a viral fusion protein, for example, a soluble viral fusion protein, that is located between a start codon (AUG in ribonucleic acids and ATG in deoxyribonucleic acids) and a stop codon (e.g., UAA (ochre), UAG (amber) or UGA (opal) in ribonucleic acids and TAA, TAG or TGA in deoxyribonucleic acids).

A vector may also include elements that facilitate cloning of the ORF or other nucleic acid element, replication, transcription, translation and/or selection. Thus, a vector may include one or more or all of the following elements: one or more promoter elements, one or more 5′ untranslated regions (5′UTRs), one or more regions into which a target nucleotide sequence may be inserted (an “insertion element”), one or more ORFs, one or more 3′ untranslated regions (3′UTRs), and a selection element. Any convenient cloning strategy known in the art may be used to incorporate an element, such as an ORF, into a vector nucleic acid.

General texts which describe molecular biological techniques, which are applicable to the present invention, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N. Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutating RSV-F protein. Additionally, cloning strategies for soluble viral fusion proteins are described more fully in WO 2012/103496, entitled EXPRESSION OF SOLUBLE VIRAL FUSION GLYCOPROTEINS IN MAMMALIAN CELLS. The disclosures of these references are hereby incorporated by reference herein in their entirety.

The compositions described herein also encompasse variants of RSV-F. The variants may contain alterations in the amino acid sequences of the RSV-F protein. The term “variant” with respect to a protein refers to an amino acid sequence that is altered by one or more amino acids with respect to a reference sequence. The variant can include “conservative” changes and/or “nonconservative” changes. Other variations can also include amino acid deletions, insertions, substitutions, or combinations thereof. Guidance in determining which amino acid residues can be substituted, inserted, or deleted without eliminating biological or immunological activity can be found using computer programs well known in the art, for example, DNASTAR software.

In one embodiment, the nucleic acids encoding a viral fusion protein provided herein can be modified by changing one or more nucleotide bases within one or more codons throughout the nucleotide sequence. As used herein, “nucleotide base” refers to any of the four deoxyribonucleic acid bases, adenine (A), guanine (G), cytosine (C), and thymine (T) or any of the four ribonucleic acid bases, adenine (A), guanine (G), cytosine (C), and uracil (U). As used herein, “codon” refers to a series of three nucleotide bases that code for a particular amino acid. Generally, each amino acid can be encoded by one or more codons. Table 1 presents substantially all codon possibilities for each amino acid.

TABLE 1
DNA Codon Table

| Amino | | Amino | |
|---|---|---|---|
| Acid | DNA Codons | Acid | DNA Codons |
| Ala/A | GCT, GCC, GCA | Leu/L | TTA, TTG, CTT, |
| | GCG | | CTC, CTA, CTG |
| Arg/R | CGT, CGC, CGA, | Lys/K | AAA, AAG |
| | CGG, AGA, AGG | | |
| Asn/N | AAT, AAC | Met/M | ATG |
| Asp/D | GAT, GAC | Phe/F | TTT, TTC |
| Cys/C | TGT, TGC | Pro/P | CCT, CCC, CCA, |
| | | | CCG |
| Gln/Q | CAA, CAG | Ser/S | TCT, TCC, TCA, |
| | | | TCG, AGT, AGC |
| Glu/E | GAA, GAG | Thr/T | ACT, ACC, ACA, |
| | | | ACG |
| Gly/G | GGT, GGC, GGA, | Trp/W | TGG |
| | GGG | | |
| His/H | CAT, CAC | Tyr/Y | TAT, TAC |
| Ile/I | ATT, ATC, ATA | Val/V | GTT, GTC, GTA, |
| | | | GTG |
| START | ATG | STOP | TAA, TGA, TAG |

In one embodiment, the nucleic acid encoding RSV-F may include one or more substitutions. The substitutions can be made to change an amino acid in the resulting protein in a non-conservative manner or in a conservative manner. A conservative change generally leads to less change in the structure and function of the resulting protein. A non-conservative change is more likely to alter the structure, activity or function of the resulting protein. In one embodiment, the nucleic acid encoding RSF-F includes one or more conservative amino acid substitutions which do not significantly alter the activity or binding characteristics of the resulting protein.

As used herein, the term “conservative substitution” refers to a substitution in which one or more amino acid residues are substituted by residues of different structure but similar chemical characteristics, such as where a hydrophobic residues is substituted by a hydrophobic residue or where an acidic residue is substituted by another acidic residue or a polar residue for a polar residue or a basic residue for a basic residue. Nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. Polar neutral amino acids include glycine, serine, threonine, cysteine, tyrosine, asparagine, and glutamine. Positively charged (basic) amino acids include arginine, lysine and histidine. Negatively charged (acidic) amino acids include aspartic acid and glutamic acid. More specific examples of conservative substitutions include, but are not limited to, Lys for Arg and vice versa such that a positive charge may be maintained; Glu for Asp and vice versa such that a negative charge may be maintained; Ser for Thr such that a free —OH can be maintained; and Gln for Asn such that a free NH₂ can be maintained. In one embodiment, the RSV-F immunogen includes one or more conserved or non-conserved amino acid substitutions. In one embodiment, the RSV-F immunogen includes one or more conserved amino acid substitutions.

The term “identical” as used herein refers to two or more nucleotide sequences having substantially the same nucleotide sequence when compared to each other. One test for determining whether two nucleotide sequences or amino acids sequences are substantially identical is to determine the percent of identical nucleotide sequences or amino acid sequences shared.

Calculations of sequence identity can be performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two aligned sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix. A set of parameters often used with a Blossum 62 scoring matrix includes a gap open penalty of 12, a gap extend penalty of 4, and a frameshift gap penalty of 5. Percent identity between two nucleotide sequences can be determined using the GAP program in the GCG software package (available at http address www.gcg.com), using NWSgapdna.CMP matrix and a gap weight of 60 and a length weight of 4.

Another manner for determining whether two nucleic acids are substantially identical is to assess whether a polynucleotide homologous to one nucleic acid will hybridize to the other nucleic acid under stringent conditions. As used herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

In the past, studies of the fusion activity of Respiratory Syncytial Virus (RSV) have been hindered by low recombinant expression levels. In particular, recombinant F protein expression levels from standard expression vectors tend to be low in comparison to the levels of F protein expression observed during RSV replication (Huang et al. (2010), “Recombinant respiratory syncytial virus F protein expression is hindered by inefficient nuclear export and mRNA processing,” Virus Genes, 40:212-221). The difference could be due to the differences between viral and recombinaint F protein expression. In general, there are two major differences between viral and recombinant F protein expression. First, transcription of the F gene during viral replication occurs in the cytoplasm, whereas transcription occurs in the nucleus during recombinant F protein expression from standard mammalian expression vectors. Export from the nucleus to the cytoplasm of viral transcripts can be problematic, even for viruses that normally replicate in the nucleus. For viral transcripts, the inhibition is thought to be a product of AU abundance, which is relatively high in comparison to mammalian transcripts. Therefore, in one embodiment, GC abundance in the F protein gene sequence can be modified to enhance transcription. (Huang et al. (2010), “Recombinant respiratory syncytial virus F protein expression is hindered by inefficient nuclear export and mRNA processing,” Virus Genes, 40:212-221).

Nucleotide sequences provided herein can be modified by changing one or more nucleotide bases within one or more codons such that the amino acid sequence of the encoded viral fusion protein is similar to the amino acid sequence of the protein encoded by the unmodified nucleotide sequence. In one embodiment, the amino acid sequence of the RSV-Fusion protein is at least about 75%, 80%, 85%, 90%, 91%, 92%, 93%, 94%, 95%, 96%, 97%, 98%, 99%, or 100% identical to the protein encoded by a unmodified wild-type RSV-F sequence, such as the RSV-F sequence shown in SEQ ID NO: 2 or the soluble RSV-F sequence shown in SEQ ID NO:7. In some embodiments, the amino acid sequence encoded by the modified nucleotide sequence is 100% identical to the amino acid sequence encoded by the unmodified wild type nucleotide sequence for RSV-F shown in SEQ ID NO: 2 or the amino acid sequence for soluble RSV-F shown in SEQ ID NO:7.

As indicated in Table 1, a subset of amino acids and the STOP codon can be encoded by at least two codon possibilities. For example, glutamate can be encoded by GAA or GAG. If a codon for glutamate exists within a nucleic acid sequence as GAA, a nucleotide base change at the third position from an A to a G will lead to a modified codon that still encodes for glutamate. Thus, a particular change in one or more nucleotide bases within a codon can still lead to encoding the same amino acid. This process, in some cases, is referred to herein as codon optimization. Provided herein are examples of nucleotide sequences for RSV-F (set forth in SEQ ID NOs: 8 and 9) that have been modified by changing one or more nucleotide bases within one or more codons wherein the resulting RSV-F amino acid sequence is identical to the amino acid sequence encoded by the unmodified nucleotide sequence (set forth in SEQ ID NO: 2). Also provided herein, for example, are nucleotide sequences for soluble RSV-F (set forth in SEQ ID NOs: 4, 5 and 6) that have been modified by changing one or more nucleotide bases within one or more codons whereby the sRSV-F amino acid sequence is identical to the amino acid sequence encoded by the unmodified nucleotide sequence (set forth in SEQ ID NO: 7).

In one embodiment, the nucleotide sequences encoding RSV-F protein, including, for example, soluble RSV-F, can be modified by changing one or more nucleotide bases within one or more codons such that a) the amino acid sequence of the encoded viral fusion protein is similar or identical to the amino acid sequence of the protein encoded by the unmodified nucleotide sequence; and b) the combined percent of guanines and cytosines (% GC) is increased in the modified nucleotide sequence compared to the unmodified nucleotide sequence. For example, the % GC in the modified nucleic acid sequence can be at least about 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 75%, 80%, 85%, 90%, 95%, or 99%. As indicated in Table 1, nucleotide base changes at the first, second and/or third codon positions can be made such that an A or a T is changed to a G or a C while preserving the amino acid and/or STOP codon assignment.

Provided herein is an example of a nucleotide sequences for RSV-F (set forth in SEQ ID NO: 9) that has been modified by changing one or more nucleotide bases within one or more codons wherein the RSV-F amino acid sequence is identical to the amino acid sequence encoded by the unmodified nucleotide sequence (set forth in SEQ ID NO: 2), and the combined percent of guanines and cytosines (% GC) is increased in the modified nucleotide sequence (58% GC) compared to the unmodified nucleotide sequence (35% GC; set forth in SEQ ID NO: 1). Also provided herein, for example, are nucleotide sequences for soluble RSV-F (e.g., set forth in SEQ ID NOs: 4, 5 and 6) that have been modified by changing one or more nucleotide bases within one or more codons such that the sRSV-F amino acid sequence is identical to the amino acid sequence encoded by the unmodified nucleotide sequence (set forth in SEQ ID NO: 7), and the combined percent of guanines and cytosines (% GC) is increased in the modified nucleotide sequences (46% GC for SEQ ID NO: 4; 51% GC for SEQ ID NO: 6; 58% GC for SEQ ID NO: 5) compared to the unmodified nucleotide sequence (35% GC; set forth in SEQ ID NO: 3).

The nucleotide sequences provided herein can be modified by changing one or more nucleotide bases within one or more codons such that a) the amino acid sequence of the encoded viral fusion protein is similar or identical to the amino acid sequence of the protein encoded by the unmodified nucleotide sequence; b) the combined percent of guanines and cytosines (% GC) is increased in the modified nucleotide sequence compared to the unmodified nucleotide sequence; and c) the overall combined percent of guanines and cytosines at the third nucleotide codon position (% GC3) is increased in the modified nucleotide sequence compared to the unmodified nucleotide sequence. In one embodiment, the % GC3 is at least about 55%, 56%, 57%, 58%, 59%, 60%, 61%, 62%, 63%, 64%, 65%, 70%, 71%, 723%, 73%, 74%, 76%, 77%, 78%, 79%, 80%, 85%, 90%, 95%, 96%, 97%, 98%, 99%, or 100%. As indicated in Table 1, most nucleotide base change possibilities reside at the third nucleotide codon position. In some embodiments, every codon, including the STOP codon, either has a G or a C in the third nucleotide codon position already or can be modified to have a G or a C at the third nucleotide codon position without changing the amino acid assignment. Thus, for any given nucleotide sequence, it is possible to have up to 100% G or C at each third nucleotide codon position (GC3) throughout the nucleotide sequence. Provided herein in an embodiment is a nucleotide sequence for RSV-F (set forth in SEQ ID NO: 9) that has been modified by changing one or more nucleotide bases within one or more codons whereby the RSV-F amino acid sequence is identical to the amino acid sequence encoded by the unmodified nucleotide sequence (set forth in SEQ ID NO: 2), and the overall combined percent of guanines and cytosines at the third nucleotide codon position is increased in the modified nucleotide sequence (100% GC3) compared to the unmodified nucleotide sequence (31% GC3; set forth in SEQ ID NO: 1). Also provided herein in an embodiment is a nucleotide sequence for sRSV-F (set forth in SEQ ID NOs: 4, 5 and 6) that has been modified by changing one or more nucleotide bases within one or more codons whereby the sRSV-F amino acid sequence is identical to the amino acid sequence encoded by the unmodified nucleotide sequence (set forth in SEQ ID NO: 7), and the overall combined percent of guanines and cytosines at the third nucleotide codon position is increased in the modified nucleotide sequences (58% GC3 for SEQ ID NO: 4; 76% GC3 for SEQ ID NO: 6; 100% GC3 for SEQ ID NO: 5) compared to the unmodified nucleotide sequence (31% GC3; set forth in SEQ ID NO: 3).

In one embodiment, the RSV-F protein, including in some embodiments, soluble RSF-F protein, has an isolated nucleic acid sequence with a GC content of at least about 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 75%, 80%, 85%, 90%, 95%, or 99% and that encodes a RSV-F protein, including for example, soluble RSV-F protein, that has an amino acid sequence that is at least about 60%, 61%, 62%, 63%, 64%, 65%, 66%, 67%, 68%, 69%, 70%, 75%, 80%, 85%, 90%, 91%, 92%, 93%, 94%, 95%, 96%, 98%, 98%, 99% or 100% identical to SEQ ID NO: 2 or SEQ ID NO:7. In another embodiment, the nucleotide sequence is 60%, 61%, 62%, 63%, 64%, 65%, 66%, 67%, 68%, 69%, 70%, 75%, 80%, 85%, 90%, 91%, 92%, 93%, 94%, 95%, 96%, 98%, 98%, 99% or 100% identical to SEQ ID NO: 3, SEQ ID NO:4, SEQ ID NO:5, SEQ ID NO:8, or SEQ ID NO:9. In one embodiment, the soluble viral fusion protein lacks a functional membrane association region. In a more particular embodiment, the soluble viral fusion protein lacks the C-terminal transmembrane region amino acids corresponding to amino acids 525 to 574 of SEQ ID NO: 2.

Also provided in certain embodiments is an isolated nucleic acid comprising a nucleotide sequence (i) having a GC content of at least about 51%, (ii) that is at least about 73% identical to SEQ ID NO: 1, and (iii) that encodes a viral fusion protein comprising an amino acid sequence at least about 90% identical to SEQ ID NO: 2.

In one embodiment, the nucleic acid sequence encoding the RSV-F protein is at least about 60% 60%, 61%, 62%, 63%, 64%, 65%, 66%, 67%, 68%, 69%, 70%, 75%, 80%, 85%, 90%, 91%, 92%, 93%, 94%, 95%, 96%, 98%, 98% or 99% identical to SEQ ID NO: 1.

Recombinant viral fusion proteins can be further modified, such as by chemical modification, or post-translational modification. Such modifications include, but are not limited to, pegylation, albumination, glycosylation, farnysylation, carboxylation, hydroxylation, hasylation, carbamylation, sulfation, phosphorylation, and other polypeptide modifications known in the art. The viral fusion proteins provided herein can be further modified by modification of the primary amino acid sequence, by deletion, addition, or substitution of one or more amino acids.

In one embodiment, the viral fusion protein is modified by post-translational glycosylation. A recombinant viral fusion protein can be fully glycosylated, partially glycosylated, deglycosylated, or non-glycosylated. In some embodiments, a recombinant viral fusion protein (e.g., RSV-F fusion protein) can have a glycosylation profile similar to, substantially identical to, or identical to the glycosylation profile of the native counterpart protein (e.g., Rixon et al., 2002 J. Gen. Virol. 83: 61-66). Recombinant viral fusion glycoproteins can include any of the multiple glycosidic linkages known in the art.

RSV-F protein suitable for use in the vaccine compositions described herein can be expressed and purified using constructs and techniques known in the art. Systems and methods for producing and purifying viral fusion proteins such as RSV-F are known, and are described more fully in WO 2012/103496, entitled EXPRESSION OF SOLUBLE VIRAL FUSION GLYCOPROTEINS IN MAMMALIAN CELLS, the disclosure of which is hereby incorporated by reference herein in its entirety.

### 5. Vaccine Formulations

As discussed previously in the background section of this application, development of an RSV vaccine has been difficult. Although vaccines have been successfully developed for other viruses, such as influenza, to date, none have been successfully developed for RSV. From a vaccine viewpoint, respiratory viruses may be divided into two principle groups-those where infection results in long-term immunity and whose continued survival requires constant mutation, and those where infection induces incomplete immunity and repeated infections are common, even with little or no mutation. Influenza virus and respiratory syncytial virus (RSV) typify the former and latter groups, respectively. (See, U. E. Power, 2008 “Respiratory syncytial virus (RSV) vaccines—Two steps back for one leap forward,” J. Clin. Virol. 41: 38-44). Consequently, although successful vaccines have been developed against influenza virus, this is not the case for RSV, despite many decades of research and several vaccine approaches.

The balance of RSV antibodies and cellular immunity required to protect against RSV disease in humans is not well understood and may vary with different age groups. For example in the elderly, cellular responses are more difficult to induce, more Th2-biased, and wane more rapidly than in young adults (Kumar R and Burns E A (2008) Age-related decline in immunity: implications for vaccine responsiveness. Expert Rev Vaccines 7: 467-479). RSV-specific T cell responses in particular decline with age (Cusi M G, et al. (2010) Age related changes in T cell mediated immune response and effector memory to Respiratory Syncytial Virus (RSV) in healthy subjects. Immun Ageing 7: 14). Elderly individuals can still succumb to severe RSV disease despite being seropositive with RSV neutralizing titers of 9-13 log 2 (Walsh E E, et al. (2004) Risk factors for severe respiratory syncytial virus infection in elderly persons. J Infect Dis 189: 233-238). The elderly have T cell defects in RSV responsiveness not seen in the young (Cusi M G, et al. (2010) Age related changes in T cell mediated immune response and effector memory to Respiratory Syncytial Virus (RSV) in healthy subjects. Immun Ageing 7: 14), and despite having similar neutralizing antibody titers to young adults (Falsey A R, et al. (1999) Comparison of respiratory syncytial virus humoral immunity and response to infection in young and elderly adults. J Med Virol 59: 221-226), are more susceptible to RSV disease following infection. These observations suggest that an effective RSV vaccine for the elderly may be required to boost both neutralizing antibodies and waning RSV specific cell mediated immunity.

As mentioned above, the elderly tend to have a Th2 bias in their immune response. The cellular immune response of a mammal includes both a T helper 1 (Th1) cellular immune response and a T helper 2 (Th2) cellular immune response. Th1 and Th2 responses are distinguishable on the basis of the cytokine profiles synthesized in each response. Type 1 T cells produce interferon gamma (IFN-γ), a cytokine implicated in the viral cell-mediated immune response. IFN-γ can therefore be referred to as a “Th1-type cytokine.” Th2 cells selectively produce interleukin 4 (IL-4), interleukin 5 (IL-5) and interleukin 13 (IL-13), which participate in the development of humoral immunity and have a prominent role in immediate-type hypersensitivity. IL-4, IL-5 and IL-13 can also be referred to as “Th2 type cytokines.” A Th1 response can also be identified by the antibody subtype produced in the response. In rodent models, a Th1 biased response has an IgG2a or IgG2b antibody titer that is greater than the IgG1 antibody titer (IgG2a and IgG2b are Th1 subtypes; IgG1 is a Th2 subtype). (Of note, in humans the converse is true; human IgG1 is a Th1 subtype and human IgG2 is a Th2 subtype, with a Th1 biased response characterized by greater IgG1 antibody titers than IgG2 antibody titers.) In both rodents and humans, a Th1 response is also marked by an increased CD8 T cell response. An imbalance in the Th1/Th2 cytokine immune response, particularly a Th2 bias in the cellular immune response of an animal, can affect pathogenesis of RSV and the severity of the infection, particularly in the lungs. Additionally, a Th2-biased primary immune response has been correlated with RSV enhanced disease (Hurwitz J L (2011) Respiratory syncytial virus vaccine development. Expert Rev Vaccines 10: 1415-1433).

Because of their prior exposure to RSV, live attenuated RSV virus vaccine would be insufficiently immunogenic in an elderly population. Pre-existing RSV immunity would likely inhibit replication of the virus vaccine and consequently limit the ability of live RSV vaccine to boost RSV immunity. Therefore, a vaccine that could prevent RSV-related illness in the elderly would address an unmet medical need in this target population.

In one embodiment, a vaccine composition is provided. In particular, the vaccine composition includes RSV-F protein as described herein. In one embodiment, the vaccine composition includes recombinantly expressed RSV-F protein as described herein. In one embodiment, the vaccine composition includes RSV soluble F protein as described herein. In one embodiment, the RSV soluble F protein lacks a C-terminal transmembrane domain. In a more particular embodiment, the RSV soluble F protein lacks a cytoplasmic tail domain.

In a more particular embodiment, the vaccine composition includes RSV soluble F protein in combination with an adjuvant. Frequently, purified protein antigens lack inherent immunogenicity, so immunogenic vaccine formulations often include a nonspecific stimulator of the immune response, known as an adjuvant. Some adjuvants affect the way in which antigens are presented. For example, in some instances an immune response is increased when protein antigens are precipitated by alum. In other instances, emulsification of antigens can prolong the duration of antigen presentation. Immunization protocols have used adjuvants to stimulate responses for many years, and as such, adjuvants are well known to one of ordinary skill in the art. Adjuvants are described in more detail in Vogel et al., “A Compendium of Vaccine Adjuvants and Excipients (2nd Edition),” herein incorporated by reference in its entirety.

Examples of known adjuvants include complete Freund's adjuvant (a non-specific stimulator of the immune response containing killed Mycobacterium tuberculosis), incomplete Freund's adjuvants and aluminum hydroxide adjuvant. Other known adjuvants include granulocyte macrophage colony-stimulating factor (GMCSP), Bacillus Calmette-Guérin (BCG), aluminum hydroxide, Muramyl dipeptide (MDP) compounds, such as thur-MDP and nor-MDP, muramyl tripeptide phosphatidylethanolamine (MTP-PE), RIBI's adjuvants (Ribi ImmunoChem Research, Inc., Hamilton Mont.), which contains three components extracted from bacteria, trehalose dimycolate (TDM) and cell wall skeleton (CWS) in a 2% squalene/Tween 80 emulsion. MF-59, Novasomes®, major histocompatibility complex (MHC) antigens are other known adjuvants.

While alum is often used as an adjuvant for vaccines, it is known for boosting humoral immunity but not for induction of effective cellular immunity (Langley J M et al. (2009) A dose-ranging study of a subunit Respiratory Syncytial Virus subtype A vaccine with and without aluminum phosphate adjuvantation in adults > or =65 years of age. Vaccine 27: 5913-5919; Falsey A R, et al. (2008) Comparison of the safety and immunogenicity of 2 respiratory syncytial virus (rsv) vaccines—nonadjuvanted vaccine or vaccine adjuvanted with alum—given concomitantly with influenza vaccine to high-risk elderly individuals. J Infect Dis 198: 1317-1326; and Kool M, et al. (2012) Alum adjuvant: some of the tricks of the oldest adjuvant. J Med Microbiol 61: 927-934). Novel adjuvant compounds incorporating Toll-like receptor (TLR)9 agonists have been shown to improve Th1-biased cellular responses to RSV vaccines in mouse models (Hancock G E, et al. (2001) CpG containing oligodeoxynucleotides are potent adjuvants for parenteral vaccination with the fusion (F) protein of respiratory syncytial virus (RSV). Vaccine 19: 4874-4882; and Garlapati S, et al. (2012) Enhanced immune responses and protection by vaccination with respiratory syncytial virus fusion protein formulated with CpG oligodeoxynucleotide and innate defense regulator peptide in polyphosphazene microparticles. Vaccine). TLR4-based adjuvants such as a Monophosphoryl Lipid A (MPL)/QS-21 combination or Protollin, a formulation of LPS complexed with meningococcal outer membrane proteins, have also been able to induce cellular IFNγ production to RSV vaccines in mice (Neuzil K M, et al. (1997) Adjuvants influence the quantitative and qualitative immune response in BALB/c mice immunized with respiratory syncytial virus FG subunit vaccine. Vaccine 15: 525-532; Cyr S L, et al. (2007) Intranasal proteosome-based respiratory syncytial virus (RSV) vaccines protect BALB/c mice against challenge without eosinophilia or enhanced pathology. Vaccine 25: 5378-5389).

Enterobacterial lipopolysaccharide (LPS) is a potent stimulator of the immune system. However, its use in adjuvants has been curtailed by its toxicity. A non-toxic derivative of LPS, monophosphoryl lipid A (MPL), produced by the removal of the core carbohydrate group and phosphate from the reducing-end glucosamine has been produced, along with a further detoxified version of MPL, produced by the removal of the acyl chain from the 3-position of the disaccharide backbone, called 3-O-deacylated monophosphoryl lipid A (3D-MPL). Another synthetic toll-like receptor (TLR)4 agonist optimized for binding to the human MD2 molecule of the TLR4 complex is a synthetic hexylated Lipid A derivative called glucopyraonosyl lipid adjuvant (GLA) (available from Avanti Polar Lipids, Inc. Alabaster, Ala.). GLA has been demonstrated to be a potent Th1-biasing adjuvant in both rodent and primate model systems (Coler R N, et al. (2010) A synthetic adjuvant to enhance and expand immune responses to influenza vaccines. PLoS One 5: e13677; and Lumsden J M, et al. (2011) Evaluation of the safety and immunogenicity in rhesus monkeys of a recombinant malaria vaccine for Plasmodium vivax with a synthetic Toll-like receptor 4 agonist formulated in an emulsion. Infect Immun 79: 3492-3500).

GLA is described in detail in U.S. Patent Publication No. 2011/0070290, entitled “Vaccine Composition Containing Synthetic Adjuvant,” the disclosure of which is hereby incorporated by reference in its entirety. As described in U.S. Patent Publication No. 2011/0070290, GLA comprises (i) a diglucosamine backbone having a reducing terminus glucosamine linked to a non-reducing terminus glucosamine through an ether linkage between hexosamine position 1 of the non-reducing terminus glucosamine and hexosamine position 6 of the reducing terminus glucosamine; (ii) an O-phosphoryl group attached to hexosamine position 4 of the non-reducing terminus glucosamine; and (iii) up to six fatty acyl chains; wherein one of the fatty acyl chains is attached to 3-hydroxy of the reducing terminus glucosamine through an ester linkage, wherein one of the fatty acyl chains is attached to a 2-amino of the non-reducing terminus glucosamine through an amide linkage and comprises a tetradecanoyl chain linked to an alkanoyl chain of greater than 12 carbon atoms through an ester linkage, and wherein one of the fatty acyl chains is attached to 3-hydroxy of the non-reducing terminus glucosamine through an ester linkage and comprises a tetradecanoyl chain linked to an alkanoyl chain of greater than 12 carbon atoms through an ester linkage. GLA has the formula

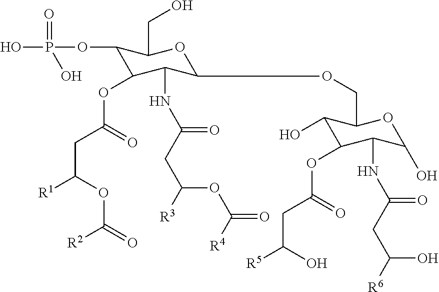

wherein R¹, R³, R⁵ and R⁶, are C₁₁-C₂₀ alkyl; and R² and R⁴ are C₁₂-C₂₀ alkyl. In some embodiments, GLA is formulated as a stable oil-in-water emulsion (SE), which is referred to herein as GLA-SE.

In one embodiment, the vaccine composition includes an adjuvant that is a Toll-like receptor (TLR) agonist. In one embodiment, vaccine composition includes an adjuvant that is a (TLR)4 agonist. Cytokines induced by TLR4 signaling, such as IL-6 and IFNγ, act as B cell growth factors and support class-switching to antibodies optimized for interactions with Fc receptors and complement (Finkelman F D, et al. (1988) IFN-gamma regulates the isotypes of Ig secreted during in vivo humoral immune responses. J Immunol 140: 1022-1027; and Nimmerjahn F and Ravetch J V (2007) Fc-receptors as regulators of immunity. Adv Immunol 96: 179-204). These cytokines additionally recruit professional antigen presenting cells, inducing MHC I molecules and antigen processing proteins upregulation to allow for better activation of T cells (Ramanathan S, et al. (2008) Antigen-nonspecific activation of CD8+ T lymphocytes by cytokines: relevance to immunity, autoimmunity, and cancer. Arch Immunol Ther Exp (Warsz) 56: 311-323). Type I IFN induced by TLR4 signaling can enhance crosspresentation of protein antigens (Durand V, et al. (2009) Role of lipopolysaccharide in the induction of type I interferon-dependent cross-priming and IL-10 production in mice by meningococcal outer membrane vesicles. Vaccine 27: 1912-1922), allowing induction of strong CD8 T cell responses to associated ovalbumin protein (Lasarte J J, et al. (2007) The extra domain A from fibronectin targets antigens to TLR4-expressing cells and induces cytotoxic T cell responses in vivo. J Immunol 178: 748-756; MacLeod M K, et al. (2011). In a more particular embodiment, vaccine composition includes an adjuvant that includes Glucopyraonsyl Lipid A (GLA). In one embodiment, the vaccine composition is formulated as a particulate emulsion. In one embodiment, vaccine composition includes an adjuvant that includes GLA in a stable oil-in-water emulsion (GLA-SE). In another embodiment, vaccine composition includes an adjuvant that includes GLA in a stabilized squalene based emulsion.

The dosage for the RSV vaccine composition can vary, for example, depending upon age, physical condition, body weight, sex, diet, time of administration, and other clinical factors and can be determined by one of skill in the art. In one embodiment, the vaccine composition is formulated as a stable aqueous suspension having a volume of at least about 50 μl, 75 μl, or 100 μl and up to about 200 μl, 250 μl, 500 μl, 750 μl or 1000 μl.

In one embodiment, at least about 1 μg, 5 μg, 10 μg, 20 μg, 30 μg or 50 μg and up to about 75 μg, 80 μg, 100 μg, 150 μg or 200 μg of RSV soluble F protein as described herein is included in the vaccine composition. In one embodiment, the vaccine composition includes RSV-F immunogen at a concentration of at least about 0.01 μg/μl, 0.05 μg/μl, 0.1 μg/μl and up to about 0.1 μg/μl, 0.2 μg/μl, 0.3 μg/μl, 0.4 μg/μl, 0.5 μg/μl or 1.0 μg/μl.

In one embodiment, the vaccine composition includes at least about 0.1 μg, 0.5 μg, 1 μg, 1.5 μg, 2 μg, or 2.5 μg and up to about 3 μg, 4 μg, 5 μg, 10 μg or 20 μg adjuvant. In one embodiment, the vaccine composition includes adjuvant at a concentration of at least about 1 ng/μl, 2 ng/μl, 3 ng/μl, 4 ng/μl or 5 ng/μl and up to about 0.1 μg/μl, 0.2 μg/μl, 0.3 μg/μl, 0.4 μg/μl or 0.5 μg/μl.

In a more particular embodiment, the adjuvant comprises GLA in a stabilized oil-in-water emulsion having a GLA concentration of at least about 1%, 2% or 3% and up to about 4% or 5%. In one embodiment, the adjuvant comprises GLA in a stabilized oil-in-water emulsion (SE), wherein GLA has a mean particle size of at least about 25 nm, 50 nm, 75 nm or 100 nm and up to about 100 nm, 125 nm, 150 nm, 175 nm or 200 nm.

In a more particular embodiment, the vaccine composition includes between about 1 μg and 100 μg RSV-sF glycoprotein in combination with between about 1 μg and 10 μg GLA in between 2% to 5% SE in a final volume between about 100 μl to about 500 μl. In a more particular embodiment, the vaccine composition is a liquid formulation that includes between about 10 μg and about 100 μg RSV-sF glycoprotein in combination with between about 1 μg and about 5 μg GLA in between 2% to 5% SE in a final volume between about 250 μl to about 500 μl. In a further embodiment, the vaccine composition is formulated for intramuscular injection and includes about 10 μg, 30 μg or 100 μg RSV-sF glycoprotein in combination with 1 μl, 2.5 μg or 5 μg GLA in 2% or 5% SE in a final volume of about 500 μl.

The amount and frequency of administration can be dependent upon the response of the host. In one embodiment, the vaccine composition is administered as a single dose. In another embodiment the vaccine composition is administered under a two dose regimen. In another embodiment, the vaccine composition is administered on a dosage schedule, for example, an initial administration of the vaccine composition with subsequent booster administrations. In one embodiment, the vaccine composition is administered under a two dose regimen in which the second dose is administered at least about 1, about 2, about 3, or about 4, weeks after the initial administration, or at least about 1, about 2, about 3, about 4, about 5 or about 6 months, after the initial administration, or at least about 1 year or longer after the initial administration. In another embodiment, the vaccine composition is administered on a dosage schedule in which a second dose is administered at least about 1, about 2, about 3, or about 4, weeks after the initial administration, or at least about 1, about 2, about 3, about 4, about 5 or about 6 months, after the initial administration, or at least about 1 year or longer after the initial administration and a third dose is administered after the second dose, for example, at least about 1, about 2, about 3, about 4, about 5, about 6 months, or about one year after the second dose.

In another embodiment, the vaccine composition includes a pharmaceutically acceptable carrier or diluent in which the immunogen is suspended or dissolved. Pharmaceutically acceptable carriers are known, and include but are not limited to, water for injection, saline solution, buffered saline, dextrose, water, glycerol, sterile isotonic aqueous buffer, and combinations thereof. For parenteral administration, such as subcutaneous injection, the carrier may include water, saline, alcohol, a fat, a wax, a buffer or combinations thereof. A thorough discussion of pharmaceutically acceptable carriers, diluents, and other excipients is presented in Remington's Pharmaceutical Sciences (Mack Pub. Co. N.J. current edition), the disclosure of which is hereby incorporated by reference in its entirety. The formulation should suit the mode of administration. In a preferred embodiment, the formulation is suitable for administration to humans, preferably is sterile, non-particulate and/or non-pyrogenic.

In other embodiments, the vaccine composition can include one or more diluents, preservatives, solubilizers, emulsifiers, and/or adjuvants. For example, the vaccine composition can include minor amounts of wetting or emulsifying agents, or pH buffering agents to improve vaccine efficacy. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

It may also be desirable to include other components in a vaccine composition, such as delivery vehicles including but not limited to aluminum salts, water-in-oil emulsions, biodegradable oil vehicles, oil-in-water emulsions, biodegradable microcapsules, and liposomes. In other embodiments, the vaccine composition can include antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Administration of the vaccine composition can be systemic or local. Methods of administering a vaccine composition include, but are not limited to, parenteral administration (e.g., intradermal, intramuscular, intravenous and subcutaneous), epidural, and mucosal (e.g., intranasal and oral or pulmonary routes or by suppositories). In a specific embodiment, compositions described herein are administered intramuscularly, intravenously, subcutaneously, transdermally or intradermally. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, intranasal or other mucosal routes of administration of a composition may induce an antibody or other immune response that is substantially higher than other routes of administration. In another embodiment, intranasal or other mucosal routes of administration of a composition described herein may induce an antibody or other immune response at the site of immunization.

### 6. Kits and Articles of Manufacture

In one embodiment a pharmaceutical pack or kit that includes one or more containers filled with one or more of the ingredients of the vaccine formulations described herein. The vaccine composition can be packaged in a hermetically sealed container such as an ampoule or sachette indicating the quantity of composition. In one embodiment, the composition is supplied as a liquid. In another embodiment, the composition is supplied as a dry sterilized lyophilized powder or water free concentrate in a hermetically sealed container, wherein the composition can be reconstituted, for example, with water or saline, to obtain an appropriate concentration for administration to a subject.

When the vaccine composition is systemically administered, for example, by subcutaneous or intramuscular injection, a needle and syringe, or a needle-less injection device can be used. The vaccine formulation can be enclosed in ampoules, disposable syringes or multiple dose vials made of glass or plastic.

### 7. Methods of Stimulating an Immune Response

In response to RSV infection, neutralizing antibodies that target the RSV-Fusion (F) and attachment (G) envelope glycoproteins are produced (Hurwitz J L (2011), “Respiratory Syncytial Virus Vaccine Development,” Expert Rev Vaccines, 10:1415-1433). F-directed neutralization responses are particularly desirable as F glycoprotein is both highly conserved between the RSV A and RSV B strains of the virus and is essential for fusion of viral and cellular membranes, a prerequisite for virus entry and replication (Maher C F, et al. (2004). Low RSV neutralizing antibody titers correlate with a higher risk of more severe RSV disease (Lee F E, et al. (2004) Experimental infection of humans with A2 respiratory syncytial virus. Antiviral Res 63: 191-196). While RSV neutralizing antibodies play a significant role in RSV immunity, providing protection to naive humans and rodents upon passive transfer, cellular responses to RSV are also believed to play a role in disease protection (Krilov L R (2002) Palivizumab in the prevention of respiratory syncytial virus disease. Expert Opin Biol Ther 2: 763-769 and Graham B S, et al. (1993) Immunoprophylaxis and immunotherapy of respiratory syncytial virus-infected mice with respiratory syncytial virus-specific immune serum. Pediatr Res 34: 167-172). The F glycoprotein contains multiple mouse and human CD8 and CD4 T cell epitopes (Olson M R and Varga S M (2008) Pulmonary immunity and immunopathology: lessons from respiratory syncytial virus. Expert Rev Vaccines 7: 1239-1255). RSV-specific CD8 T cell responses are detected in seropositive human adults (Cusi M G, et al. (2010) Age related changes in T cell mediated immune response and effector memory to Respiratory Syncytial Virus (RSV) in healthy subjects. Immun Ageing 7: 14) and play an important role in clearing virus-infected cells and resolving RSV infection in animal models (Bangham C R, et al. (1985) Cytotoxic T-cell response to respiratory syncytial virus in mice. J Virol 56: 55-59; Srikiatkhachorn A and Braciale T J (1997) Virus-specific CD8+T lymphocytes downregulate T helper cell type 2 cytokine secretion and pulmonary eosinophilia during experimental murine respiratory syncytial virus infection. J Exp Med 186: 421-432; Hussell T, et al. (1997) CD8+ T cells control Th2-driven pathology during pulmonary respiratory syncytial virus infection. Eur J Immunol 27: 3341-3349; and Munoz J L, et al. (1991) Respiratory syncytial virus infection in C57BL/6 mice: clearance of virus from the lungs with virus-specific cytotoxic T cells. J Virol 65: 4494-4497). RSV-specific CD4 T cell responses promote both B cell antibody production and CD8 responses, with Th1-type CD4 responses promoting CD8 responses more effectively than Th2-type responses (Hurwitz J L (2011), “Respiratory Syncytial Virus Vaccine Development,” Expert Rev Vaccines, 10:1415-1433).

In one embodiment, a method for administering an immunologically effective amount of a composition containing an immunogenic RSV-F protein to a subject (such as a human or animal subject) is provided. In one embodiment, a method in which a vaccine composition that includes an immunogenic RSV-F protein and at least one adjuvant is administered to a mammal is provided. In one embodiment, RSV-F includes soluble RSV-F (also designated as RSV-sF). In one embodiment, the adjuvant is GLA. In a more specific embodiment, the adjuvant is GLA-SE. In one embodiment, a method for eliciting an immune response against RSV is provided. In one embodiment, the immune response is humoral. In another embodiment, the immune response is cell-mediated. In one embodiment, the method induces a protective immune response to RSV infection or at least one symptom thereof. In a further embodiment a method for preventing or treating a disease by administering to a patient having said disease, or at risk of contracting said disease, a therapeutically, or prophylactically, effective amount of the vaccine composition is provided. In one embodiment, the disease is a disease of the respiratory system, for example, a disease is caused by a virus, in particular RSV.

In one embodiment, the vaccine composition is capable of eliciting in a host at least one immune response. In one embodiment, the immune response is selected from a T_{H1}-type T lymphocyte response, a T_{H2}-type T lymphocyte response, a cytotoxic T lymphocyte (CTL) response, an antibody response, a cytokine response, a lymphokine response, a chemokine response, and an inflammatory response. In one embodiment, the vaccine composition is capable of eliciting in a host at least one immune response that is selected from (a) production of one or a plurality of cytokines wherein the cytokine is selected from interferon-gamma (IFN-γ), tumor necrosis factor-alpha (TNF-α), (b) production of one or a plurality of interleukins wherein the interleukin is selected from IL-1, IL-2, IL-3, IL-4, IL-6, IL-8, IL-10, IL-12, IL-13, IL-16, IL-18 and IL-23, (c) production one or a plurality of chemokines wherein the chemokine is selected from MIP-1α, MIP-113, RANTES, CCL4 and CCL5, and (d) a lymphocyte response that is selected from a memory T cell response, a memory B cell response, an effector T cell response, a cytotoxic T cell response and an effector B cell response.

In one embodiment, the vaccine composition is able to provide an immune response that preferentially includes production of Th1-type cytokines, such as IFNγ (Th1 biased) as compared to Th2 biased cytokines such as IL-5/IL-4. In one embodiment, administration of the vaccine composition enhances a Th1 biased cellular immune response in a mammal that has been previously exposed to RSV. In one embodiment, the ratio of Th1/Th2 cellular immune response is at least about 1:1, 1.1:1, 1.2:1, 1.3:1, 1.4:1, 1.5:1, or 2:1. In one embodiment, a method of inducing or enhancing a Th1-type F protein specific CD4 or CD8 response is provided. In one embodiment, administration of an adjuvanted vaccine composition described herein induces between about 49 and about 150 F protein specific CD4 T cell spot forming units (SFU)/10⁶ total live cells, or about a 5 to 10 fold increase as compared to an unadjuvanted vaccine composition. In another embodiment, administration of an adjuvanted vaccine composition described herein induces between about 1069 and 3172 F specific CD8 T cell SFU/10⁶ total live cells, or about a 10 to 20 fold increase as compared to an unadjuvanted composition. In another embodiment, a method of inducing cellular IFNγ producing T cell response (i.e., a Th1 type cytokine) is provided. In one embodiment, administration of an adjuvanted vaccine composition provides at least a 45 fold increase in IFNγ producing T cells as compared to an unadjuvanted composition.

In one embodiment, a method of inducing neutralizing antibodies against RSV in a mammal is provided. In one embodiment, the RSV neutralizing antibody titers are greater than a titer selected from 6 Log₂, 6.5 Log₂, 7.0 Log₂, 7.5 Log₂, 8.0 Log₂, 8.5 Log₂, 9.0 Log₂, 9.5 Log₂, 10.0 Log₂, 10.5 Log₂, 11.0 Log₂, 11.5 Log₂, 12.0 Log₂, 12.5 Log₂, 13.0 Log₂, 13.5 Log₂, 14.0 Log₂, 14.5 Log₂, and 15.0 Log₂. In one embodiment, the RSV neutralizing antibody titers after administration of the vaccine composition comprise serum IgG titers that are between about 10 fold and about 200 fold greater compared serum IgG titers before administration, or at least about 10, 25, 50, 75, 100 fold greater and up to about 100, 150 or 200 fold greater. In one embodiment, the RSV neutralizing antibody titers after administration of the vaccine composition comprise serum IgG titers that are at least about 10 fold and up to about 200 fold greater compared serum IgG titers before administration.

In one embodiment, administration of the vaccine composition induces mucosal (IgA) and systemic antibody (IgG, IgG1, IgG2a, and IgG2b) responses which are able to neutralize RSV. The IgG1/IgG2a ratios indicated a Th₁ biased antibody response since IgG2a>IgG1.

In one embodiment, administration of the vaccine composition results in a reduction in RSV viral titers. In one embodiment, RSV viral titers are reduced between about 50 and about 1000 fold, or reduced at least about 50, 100, 250, 500 fold and up to about 500 or 1000 fold. In one embodiment, RSV viral titers are less than 2 log 10 pfu/gram after administration of the vaccine composition.

### EXAMPLES

### Example 1a and 1b

### Naive BALB/c Mice and Cotton Rats

BALB/c mice and cotton rats are two well-characterized rodent models of RSV infection. In this example, these two models were used to evaluate the immunogenicity of intramuscularly (IM) administered RSV vaccine candidates, which included purified soluble F (sF) protein formulated with TLR4 agonist glucopyranosyl lipid A (GLA), stable emulsion (SE), glucopyraonosyl lipid A stable emulsion (GLA-SE), or alum adjuvants. Purified sF proteins lacking transmembrane and cytoplasmic tail domains (Huang K, et al. (2010) Recombinant respiratory syncytial virus F protein expression is hindered by inefficient nuclear export and mRNA processing. Virus Genes 40: 212-221) were formulated with GLA, SE, or GLA-SE and compared in vaccine performance to sF formulated with alum or left unadjuvanted. The results demonstrate that, while each intramuscularly-administered adjuvanted RSV sF vaccine formulation induced RSV neutralizing titers and conferred protective immunity against viral replication, only sF+GLA-SE vaccines primed IFNγ-producing T cell responses in both BALB/c and cotton rat models. In the BALB/c mouse, these T cell responses were primarily CD8+, could traffic to the lung, and correlated with a Th1-biased cytokine response. RSV sF with GLA-SE adjuvant was found to be the best vaccine formulation in these studies, improving key immunological and protection readouts over unadjuvanted RSV sF while avoiding Th2-associated lung pathologies following viral infection.

Full protection from RSV challenge, robust serum RSV neutralizing responses, and anti-F IgG responses were induced by all RSV sF vaccine formulations in the murine model. When formulated with the adjuvant GLA-SE, the RSV sF protein vaccine induced F-specific Th1-biased humoral and cellular responses. In mice, both F-specific CD4 and CD8 T cell responses were identified. F-specific polyfunctional CD8 T cells trafficked to the mouse lung following RSV challenge, where viral clearance was achieved without Th2-mediated immune sequelae. In cotton rats, sF+GLA-SE induced robust neutralizing antibodies, F-specific IFNγ T cell responses, and full protection with no evidence of lung histopathology.

The data herein demonstrates that a protein subunit vaccine that includes RSV sF and GLA-SE can induce robust humoral and cellular responses to RSV, enhancing viral clearance via a Th1 immune-mediated mechanism. An adjuvanted RSV vaccine that induces robust neutralizing antibody and T cell responses may benefit populations at risk for RSV disease.

Vaccine Components

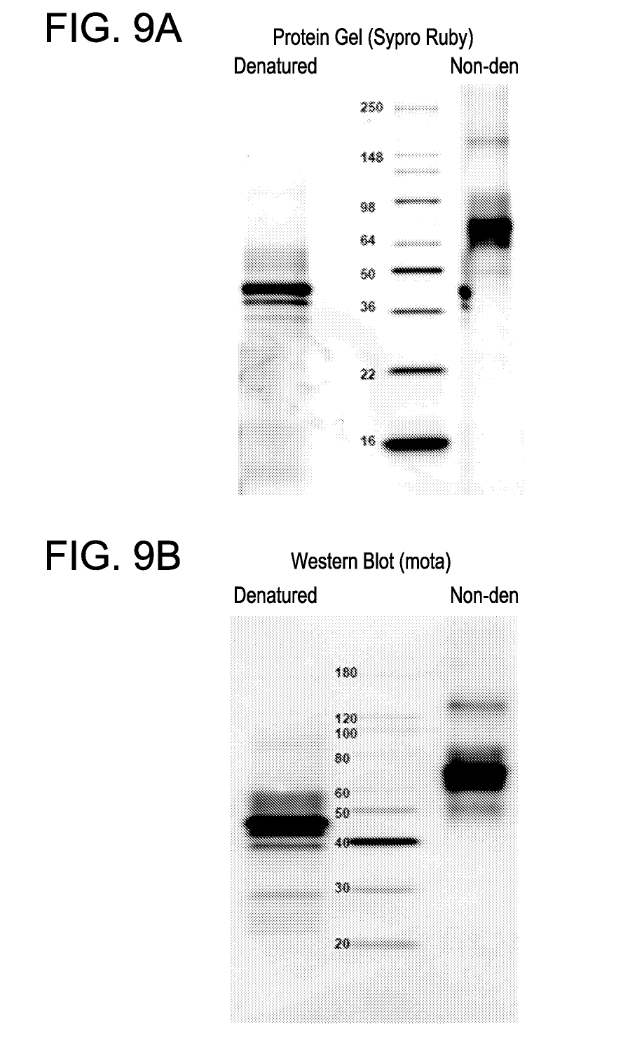

An RSV soluble F (sF) protein containing amino acids 1-524 of the RSV A2 F sequence and lacking the transmembrane domain (Huang K, et al. (2010) Recombinant respiratory syncytial virus F protein expression is hindered by inefficient nuclear export and mRNA processing. Virus Genes 40: 212-221) was immuno-affinity purified with the RSV-F-specific mAb, palivizumab (MedImmune, Inc.) from the supernatants of stably transfected Chinese Hamster Ovary (CHO) cells. SDS-PAGE and western blot analysis indicated that affinity-purified RSV sF protein was >95% pure, running under reducing conditions as both a ˜50 kD (F1) and ˜20 kD (F2) band (FIGS. 9A and B). Cryoimaging results indicated the sF protein forms both trimers and larger multimers, while ELISA binding studies confirm that it contains intact site A, B, and C neutralizing epitopes (data not shown). RSV sF was quantified by Bradford assay and used both for immunizations and coating in ELISA assays.

Adjuvants used in this study included alum (aluminum hydroxide) obtained as Alhydrogel (Accurate Chemical and Scientific, NJ). Alum was used at 100 g per vaccine dose, and adsorbed to protein by 30 minutes of mixing at 22 degrees. GLA, SE, and GLA-SE were obtained from Immune Design Corporation (Seattle, Wash.) and have been previously described (Anderson R C, et al. (2010) Physicochemical characterization and biological activity of synthetic TLR4 agonist formulations. Colloids Surf B Biointerfaces 75: 123-132). GLA in an aqueous formulation was used at 5 g per vaccine dose. SE is a stabilized squalene-based emulsion with a mean particle size of ˜100 nm that was used at a 2% concentration. Except where otherwise noted, GLA-SE was used at a dose of 5 μg GLA in 2% SE. All vaccine formulations were prepared within 24 hours of inoculation.

RSV A2 strain (ATCC) was used for immunization and challenge. Virus was propagated in Vero cells grown with EMEM. Viral supernatants were centrifuged to remove cellular debris, stabilized with 1×SP (0.2 M sucrose, 0.0038 M KH₂PO₄, and 0.0072 M KH₂PO₄) and snap frozen in aliquots at ˜80 degrees Celsius until use. Virus titers were determined by plaque assay on Vero cell monolayers as described by Tang R S, et al. (2004) Parainfluenza virus type 3 expressing the native or soluble fusion (F) Protein of Respiratory Syncytial Virus (RSV) confers protection from RSV infection in African green monkeys. J Virol 78: 11198-11207.

Vaccination and Challenge

7-10 week old female BALB/c mice (Charles River Laboratories, Hollister, Calif.) and 6-8 week old female cotton rats (Harlan Laboratories, Indianapolis, Ind.) were housed under pathogen-free conditions. Groups of mice were anesthetized and immunized intramuscularly twice, two weeks apart, with placebo (PBS) or RSV sF−/+adjuvant in a 100 μl volume. Unless otherwise indicated, RSV sF was given at a dose of 0.3 μg, which had been determined from a titration study to provide suboptimal protection in the absence of adjuvant. The most effective doses of each adjuvant were chosen from preliminary studies (data not shown). Positive controls were infected intranasally once at DO with 10⁶ PFU RSV-A2. All vaccines were well-tolerated upon administration, with no injection site reactions in any group. Sera were obtained from retro orbital blood collection at day 14 and 28 post immunization, separated from whole blood and stored at −20° C. until evaluated. Mice were inoculated intranasally with 10⁶ PFU of live RSV A2 virus in 100 μl volume at day 28 of the study. Spleens were harvested for T cell assays at 14 days post final immunization or at 4 days post challenge. Viral titers were quantified at 4 days after challenge in individual lung homogenates by plaque assay. Individual lung lobes from each animal were reserved and inflated with PBS+4% paraformaldehyde for up to 1 week, then dehydrated and embedded in paraffin for histopathology studies. Cotton rat studies were similarly designed, except that the animals were boosted three weeks following the initial priming and challenged three weeks following the booster vaccine.

Pulmonary RSV Quantitation by Plaque Titration

Fresh lungs excised from euthanized mice or cotton rats were weighed and homogenized in OptiMEM (Invitrogen) supplemented with 1×SP buffer using an OMNI tissue homogenizer with disposable heads (Omni International, Kennesaw, Ga.). Homogenates were clarified by centrifugation. Virus titers were determined by plaque assay on Vero cell monolayers as described by Tang R S, et al. (2004) Parainfluenza virus type 3 expressing the native or soluble fusion (F) Protein of Respiratory Syncytial Virus (RSV) confers protection from RSV infection in African green monkeys. J Virol 78: 11198-11207. Briefly, serial dilutions of freshly prepared lung homogenates were added to Vero cells in 6 well plates, allowed to infect for 1 hr, then overlaid with 1% methyl cellulose/EMEM and incubated for 5-7 days to allow plaque formation. Overlay was removed, cells were methanol-fixed, and plaques were visualized by staining with goat anti-RSV (Millipore, Billerica, Mass.), followed by HRP-rabbit anti-goat antibody and AEC (Dako, Glostrup, Denmark).

Serum IgG, IgG1, IgG2a and IgA ELISA

RSV-F-specific IgG antibodies were assessed using standard ELISA techniques. High binding 96 well plates were coated with purified RSV sF. After blocking, serial dilutions of serum were added to plates. Bound antibodies were detected using HRP-conjugated goat anti-mouse IgG, IgG1, or IgG2a (Jackson ImmunoResearch, West Grove, Pa.) and developed with 3,3′,5,5′-tetramethylbenzidine (TMB, Sigma, St. Louis, Mo.). RSV-F-specific IgA antibodies were detected using HRP-conjugated goat anti-mouse IgA (Invitrogen, Grand Island, N.Y.). The signal was amplified using ELAST ELISA amplification Kit (Perkin Elmer, Waltham, Mass.) and detected with TMB. Absorbance was measured at 450 nm on a SpectraMax plate reader and analyzed using SoftMax Pro (Molecular Devices, Sunnyvale, Calif.). Titers are reported as log₂ endpoint titers using a cutoff of 3× the mean of the blank wells.

RSV Micro-Neutralization Assay

RSV neutralizing antibody titers in heat-inactivated mouse sera at indicated timepoints were measured using a GFP-tagged RSV A2 micro-neutralization assay as previously described (Bernstein D I, et al. (2012) Phase 1 study of the safety and immunogenicity of a live, attenuated respiratory syncytial virus and parainfluenza virus type 3 vaccine in seronegative children. Pediatr Infect Dis J 31: 109-114). Briefly, confluent Vero cell monolayers were infected with 500 PFU of virus alone or virus pre-mixed with serially diluted serum samples, then incubated at 33° C. and 5% CO₂ for 22 hrs. Plates were washed of free virus and GFP fluorescent viral foci were enumerated using the IsoCyte image scanner (Blueshift, Sunnyvale, Calif.). Neutralizing titers were expressed as the log₂ reciprocal of the serum dilution that resulted in a 50% reduction in the number of fluorescent foci (EC₅₀ titers) as calculated using a 4-parameter curve fit algorithm.

Cell Isolation

Individual spleens were disrupted through a 100 micron nylon filter (Falcon) at the indicated harvest times. Viability of red blood cell depleted splenocytes was determined by ViCell and cells were resuspended at 10×10⁶ viable cells/mL in RPMI 1640 supplemented with 5% FCS, penicillin-streptomycin, 2 mM L-glutamine and 0.1% 3-mercaptoethanol (cRPMI-5) prior to use.

Lung leukocytes were isolated from enzyme dispersed lung tissue at the indicated harvest times. Lungs were excised, washed in PBS, minced, and incubated for 45 minutes in RMPI 5% FCS, 1 mg/mL collagenase (Roche Applied Science) and 30 μg/mL DNase (Sigma, St Louis Mo.) prior to disruption through a 100 micron nylon filter (Falcon). Cells were washed and resuspended in cRPMI-5 and total viable cell counts were determined by ViCell.

Cytokine Profiling

For cytokine restimulation assays, splenocytes were incubated in 96 well plates with either medium alone (cRPMI-5) or with the pair of RSV-F derived MHC II (I-Ed)-binding peptides GWYTSVITIELSNIKE (SEQ ID NO: 10) and VSVLTSKVLDLKNYI (SEQ ID NO: 11) (Olson M R, Varga S M (2008) Pulmonary immunity and immunopathology: lessons from respiratory syncytial virus. Expert Rev Vaccines 7: 1239-1255) (5 μg/mL each) for 72 hours. Supernatants were clarified by centrifugation and stored at −80 degrees Celsius until evaluated.

Mouse cytokine/chemokine multiplex kits designed to include IFNγ, IL-5, IL-13, IL-17 and eotaxin (Millipore, Billerica, Mass.) were used to evaluate restimulated splenocyte supernatants and fresh lung homogenates. Lung homogenates were clarified by centrifugation prior to use. Assays were performed following manufacturer's instructions and plates were analyzed on a Luminex reader (Bio-Rad, Hercules, Calif.). F-specific splenic cytokine production was determined by subtracting media alone values from F stimulated values.

ELISPOT Assays

Mabtech (Cincinnati, Ohio) murine IFNγ ELISPOT kits were used for mouse ELISPOT assays. Pre-coated microtiter plates were blocked with cRPMI-5 prior to addition of cells and stimulants. 250,000 cells/well were incubated on blocked coated plates for 36-48 hours in triplicate with media alone, MHC II (I-E^{d})-binding peptides GWYTSVITIELSNIKE (SEQ ID NO:10) and VSVLTSKVLDLKNYI (SEQ ID NO:11) (Olson M R, Varga S M (2008) Pulmonary immunity and immunopathology: lessons from respiratory syncytial virus. Expert Rev Vaccines 7: 1239-1255)(5 μg/mL each), MHC I (H2-K^{d}) binding peptide, KYKNAVTEL (SEQ ID NO: 12) (Olson M R (2008), or ConA (5 μg/mL) as a positive control. Following incubation cells were washed away, plates were incubated with included biotinylated anti-murine IFNγ followed by SA-HRP following the kit protocol, and spots were detected with included TMB reagent. Plates were read and analyzed using a CTL ImmunoSpot reader and software (Cellular Technology Ltd).

Paired antibodies for cotton rat IFNγ (#DY565) or IL-4 (#DY584) obtained in R&D DuoSet ELISA Systems were used in ELISPOT assay formats for the evaluation of cotton rat cellular immune responses. 96 well PVDF plates (Millipore, Billerica, Mass.) were coated overnight with kit provided capture antibody (anti-IFNγ or anti-IL-4, respectively) at 10 μg/mL in PBS. Plates were blocked with cRPMI-5 for 2 hours. Cells were then incubated on blocked coated plates in cRPMI-5 for 36-48 hours in triplicate with media, RSV sF (2 g/mL), or ConA (5 μg/mL) as a positive control. Following incubation cells were washed away, plates were incubated with included biotinylated detection antibody (1 μg/mL in PBS+1% BSA) followed by streptavidin-HRP (Mabtech, Cincinnati, Ohio) and 3-amino-9-ethylcarbazole (AEC, Vector Labs, Burlingame, Calif.). Plates were read and analyzed using a CTL ImmunoSpot reader and software (Cellular Technology Ltd).

Flow Cytometry Analysis

Red blood cell depleted splenocytes and lung leukocytes were distributed in 96well microtiter plates at 1·10⁶ cells/well with media alone, MHC I (H2-K^{d}) binding F peptide KYKNAVTEL (SEQ ID NO: 12) (10 g/mL), MHC I (H2-K^{d}) binding M2 peptide SYIGSINNI (SEQ ID NO:13) (10 g/mL), or ConA as a positive control. Cells were incubated at 37° C. in 5% CO₂ for 5-6 hrs, with Brefeldin A added an hour into the stimulation to block cytokine secretion. Cells were stained for viability with LIVE/DEAD violet, then with CD3-PerCP-Cy5.5, CD8-PE-Cy7, and CD19-APC-Cy7. Following fixation with 2% paraformaldehyde and permeabilization with CellPerm (BD Bioscience), cells were stained with IFNγ-APC, IL-2 FITC, and TNFα-PE. Cells were analyzed on a LSR 2 (BD Biosciences), collecting 10,000 CD8+ events.

Lung Histopathology

Lung sections (5 micron) were prepared using a microtome from paraffin-embedded formalin-fixed lung lobes harvested at day 4 post RSV challenge. Sections stained with hematoxylin and eosin were digitally scanned and examined by a licensed pathologist. Lung sections were evaluated for pulmonary lesion characteristics such as presence of bronchiolar hyperplasia, alveolitis, eosinophilic infiltrate and infiltration of the peribronchiolar/perivascular spaces.

Statistics

Data was analyzed using Prism GraphPad software. Data shown is representative of two or more experiments. All data is expressed as arithmetic mean+_standard error of the mean (SEM). Statistical significance was calculated by One way ANOVA followed by a Tukey post test with a cutoff of p<0.05.

Results

1. Adjuvanted RSV sF Subunit Vaccines Confer Protective Immunity in BALB/c Mice, with GLA-SE Adjuvanted RSV sF Inducing a Th1-Biased Protective Immunity

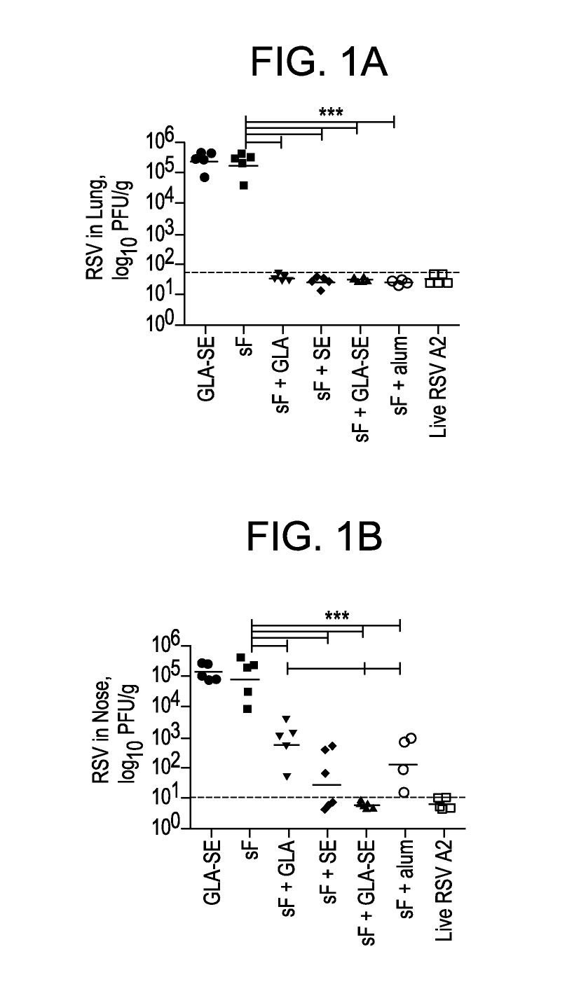

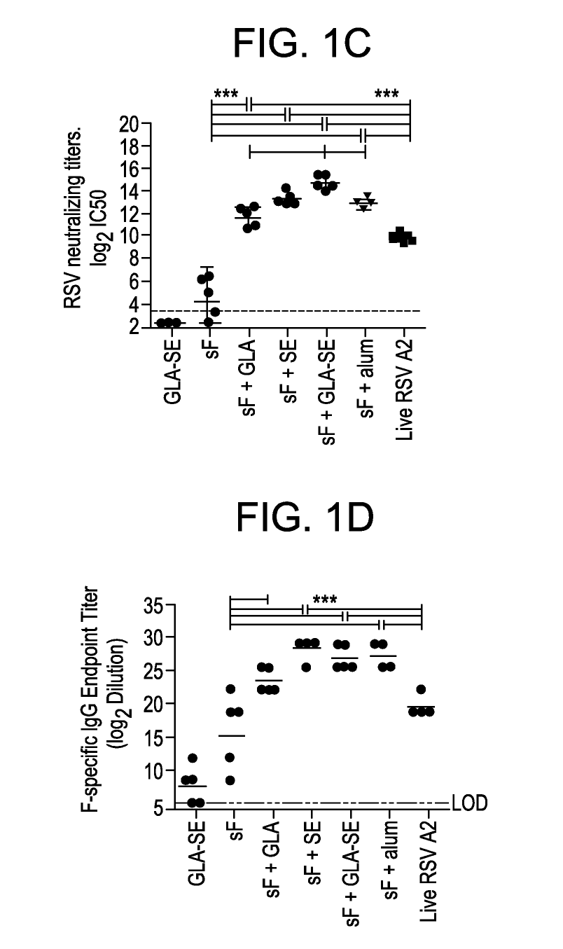

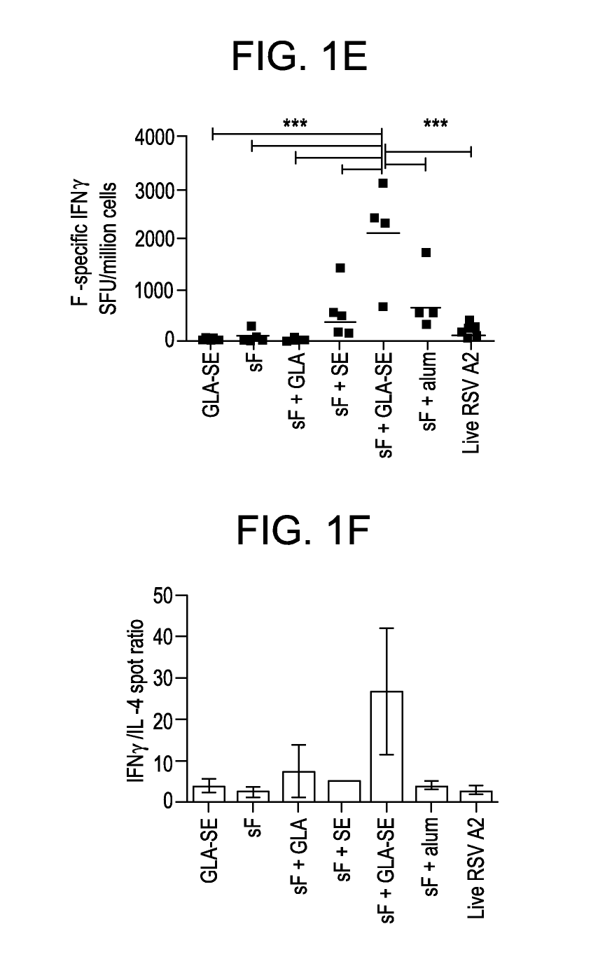

Cohorts of BALB/c mice were intramuscularly immunized with two doses of RSV sF subunit vaccines given without adjuvant or adjuvanted with alum, GLA, SE, or GLA-SE. Following challenge with RSV A2 virus, lung viral titers were quantified. All vaccines provided significant lung viral titer decreases compared to PBS controls, which had a mean lung viral titer of 3.8 log₁₀ pfu/gram (FIG. 1A). Full protection was considered a 100-fold reduction compared to the PBS negative control group. Immunization with unadjuvanted RSV sF provided partial lung protection to mice, with 4/7 animals having detectable lung viral titers ranging from 2.3-3.0 log₁₀ pfu/gram, while the adjuvanted RSV sF vaccines provided full lung protection, with mean viral titers below 1.8 log₁₀ pfu/gram consistent with that seen in the live RSV A2 immunized group.

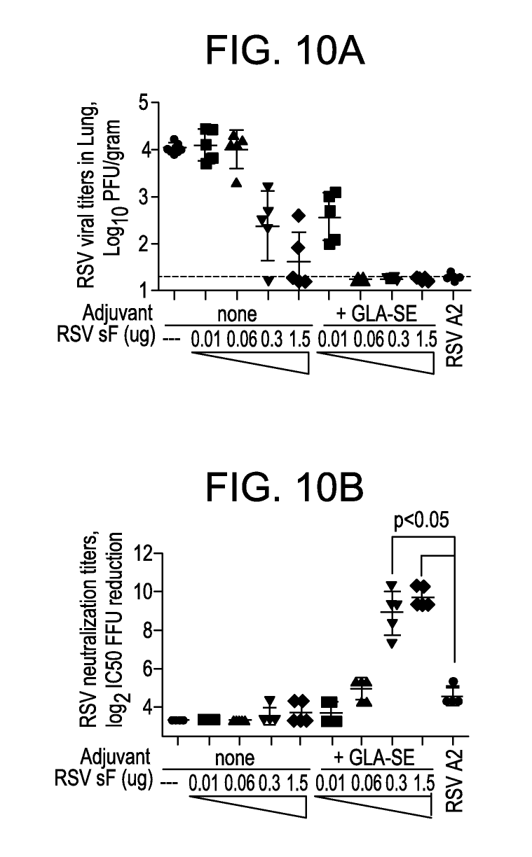

Serum RSV neutralizing titers prior to challenge were significantly enhanced with all RSV sF adjuvanted vaccines. GLA-SE, alum and SE adjuvanted RSV sF vaccines achieved the highest RSV neutralizing titers of 7.7 log₂, 8.1 log₂ and 8.1 log₂, respectively, at day 28 (FIG. 1B). These titers were 16-fold greater than those achieved by immunization with unadjuvanted RSV sF (4.1 log₂). In contrast GLA adjuvanted RSV sF achieved a respectable but significantly lower 6.3 log₂ neutralizing titer. While both unadjuvanted RSV sF and intranasal infection with live RSV A2 virus induced detectable serum neutralizing titers (4.1 log₂ and 4.6 log₂ respectively), these responses were not significantly above the limit of detection found with the PBS negative control group. ELISA titers for total serum F-specific IgG and F-specific IgA showed a similar trend (FIG. 10).

Supernatants from restimulated splenocytes (n=3 per group) harvested at 4 days post challenge were evaluated to determine the cytokine production profile of F-specific CD4+ T cells induced by each vaccine formulation. Following restimulation with MHC II (I-E^{d})-binding RSV-F derived peptides (Olson M R and Varga S M (2008) Pulmonary immunity and immunopathology: lessons from respiratory syncytial virus. Expert Rev Vaccines 7: 1239-1255), IFNγ was evaluated as the prototypical Th1-type cytokine, IL-5 and IL-13 as representative Th2-type cytokines and IL-17 as a Th17-type cytokine. As expected, while restimulated splenocytes from PBS control animals demonstrated no F-specific cytokine production, those from intranasally RSV infected mice demonstrated a weak IFNγ-dominated response (FIG. 1C). The RSV sF+GLA-SE vaccine group induced a strong RSV-F-specific response dominated by IFNγ, indicative of a Th1-type response. In contrast, the RSV sF+GLA group demonstrated a balanced F-specific response that included Th1, Th2, and Th17 cytokines, while RSV sF, RSV sF+SE, and RSV sF+alum groups demonstrated a Th2-type response characterized by IL-5 and IL-13 cytokines.

Since IFNγ promotes class-switching of antibodies from IgG1 to IgG2a in the mouse (Xu W and Zhang J J (2005) Stat1-dependent synergistic activation of T-bet for IgG2a production during early stage of B cell activation. J Immunol 175: 7419-7424), we also evaluated the isotypes of F-specific antibodies from each animal. Only two groups demonstrated F-specific IgG2a>IgG1 titers: the RSV sF+GLA-SE vaccinated group and the group primed with an infection with RSV A2 (FIG. 1D), both of which had an IFNγ dominated response to MHC II-derived F peptides. RSV sF+GLA-SE induced significantly more F-specific IgG2a antibodies than did RSV sF alone or RSV sF+alum.

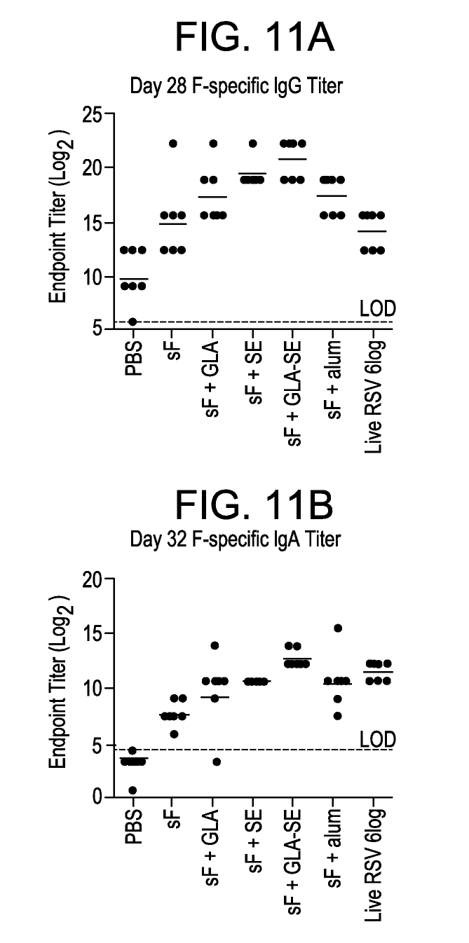

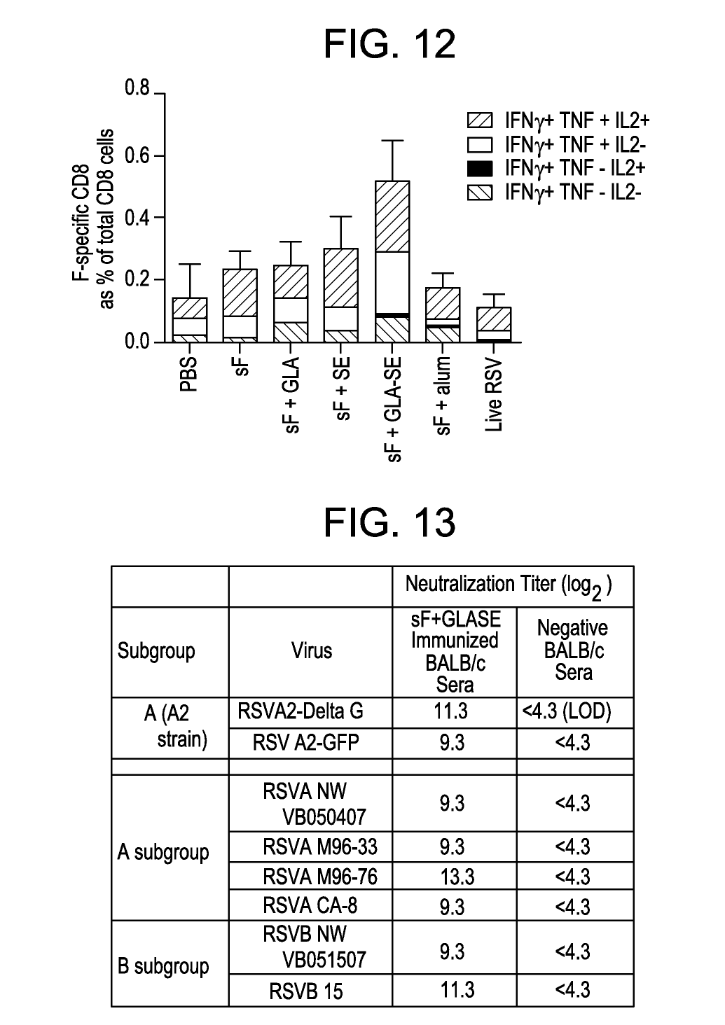

Th1-type responses to a vaccine such as those seen with RSV sF+GLA-SE may support the development of strong CD8 T cell responses. Thus, CD8 T-cell responses to vaccination were evaluated in representative animals from each vaccine group at Day 32 by restimulation with an immunodominant MHC I (H2-K^{d}) binding F-derived peptide (Olson M R, Varga S M (2008) Pulmonary immunity and immunopathology: lessons from respiratory syncytial virus. Expert Rev Vaccines 7: 1239-1255). F-specific CD8 IFNγ ELISPOT counts in the PBS control group were near undetectable, while those in the unadjuvanted RSV sF group were ˜30 spot forming units (SFU)/million cells (FIG. 1E). In contrast, F-specific CD8 IFNγ ELISPOT responses were significantly greater in the RSV sF+GLA-SE vaccine group compared to RSV sF (mean: 684, a 23-fold increase relative to unadjuvanted RSV sF). While F-specific CD8 IFNγ responses were slightly higher with other adjuvanted RSV sF vaccine formulations, these were not significant compared to unadjuvanted RSV sF. Live RSV infection generated a weak F-specific CD8 IFNγ ELISPOT response of only 100 SFU, which was not unexpected as the immunodominant response to RSV A2 in the BALB/c mouse is against an M2-derived peptide (Olson M R and Varga S M (2008) Pulmonary immunity and immunopathology: lessons from respiratory syncytial virus. Expert Rev Vaccines 7: 1239-1255). To evaluate the cytolytic potential of these responding cells, we evaluated F-specific Granzyme B secretion by ELISPOT. Only splenocytes from mice that had received sF+GLA-SE vaccines had F-specific Granzyme B responses (mean 197) significantly greater than observed in those given sF alone (mean 18) (FIG. 1F). Since polyfunctional T cells that co-express IFNγ, TNFα (an effector cytokine) and IL-2 (a cytokine associated with proliferation) are reported to be the most effective at viral clearance, followed by T cells that co-express both IFNγ and TNFα (Seder R A, et al. (2008) T-cell quality in memory and protection: implications for vaccine design. Nat Rev Immunol 8: 247-258), we additionally evaluated F-specific CD8 T cells by intracellular cytokine staining. The RSV sF+GLA-SE vaccine group had the highest numbers of both triple positive and IFNγ TNF double positive cells (FIG. 11).

These results demonstrate that while RSV sF is immunogenic alone, formulation of RSV sF with an adjuvant induces higher titer neutralizing antibodies in naive animals, and formulating RSV sF with GLA-SE generates a Th1-biased immunity that primes for a strong F-specific CD8 T cell response that may contribute to improved viral clearance.

2. CD8 T Cell Responses Primed by GLA-SE Adjuvanted RSV sF Vaccines are Robust

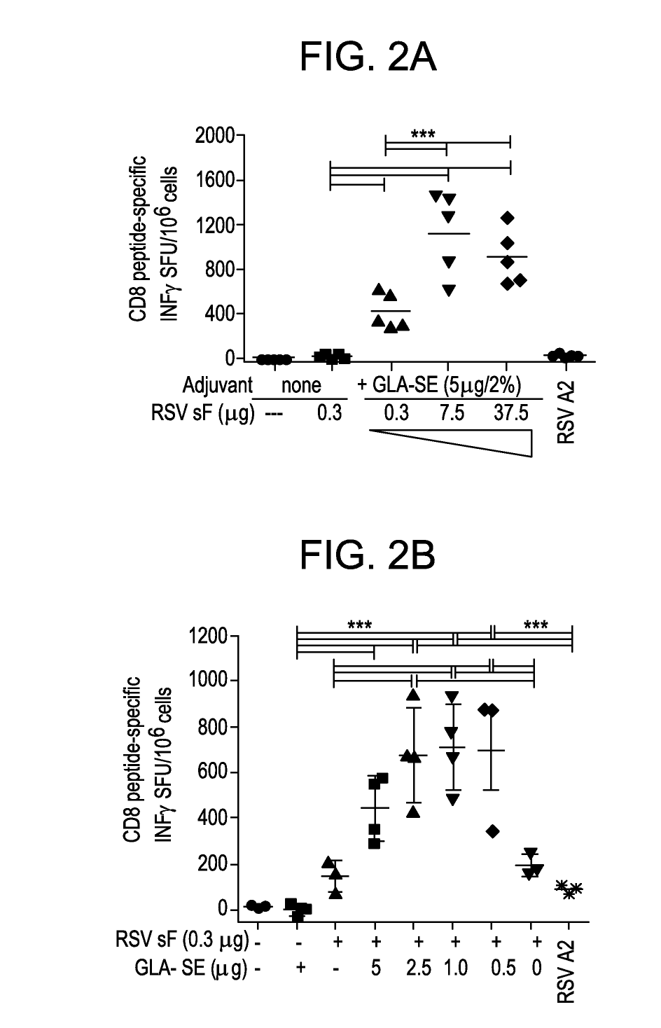

CD8 T cell responses observed post-challenge following a prime/boost vaccination with RSV sF+GLA-SE were robust over a range of antigen and adjuvant doses. Animals that received 0.3, 7.5, or 37.5 μg RSV sF given with a fixed dose of GLA-SE (5 g/2%) all generated strong F-specific CD8 T cells compared to PBS controls as detected by ELISPOTs conducted 4 days post RSV challenge (FIG. 2A). Higher absolute spot counts were found in animals given higher doses of RSV sF. Animals that received 0.3 μg RSV sF given with GLA-SE at a range of doses (5 μg, 2.5 μg, 1 μg, or 0.5 μg in 2% SE) also demonstrated significantly enhanced numbers of F-specific CD8 T cells compared to either the PBS control group or the adjuvant alone control group at 4 days post challenge (FIG. 2B). An adjuvant dose of 1-2.5 μg GLA in 2% SE was sufficient for optimal splenic T cell responses.

3. GLA-SE Adjuvanted RSV sF Vaccines Induce F-Specific CD4 and CD8 T Cell Responses without Viral Exposure

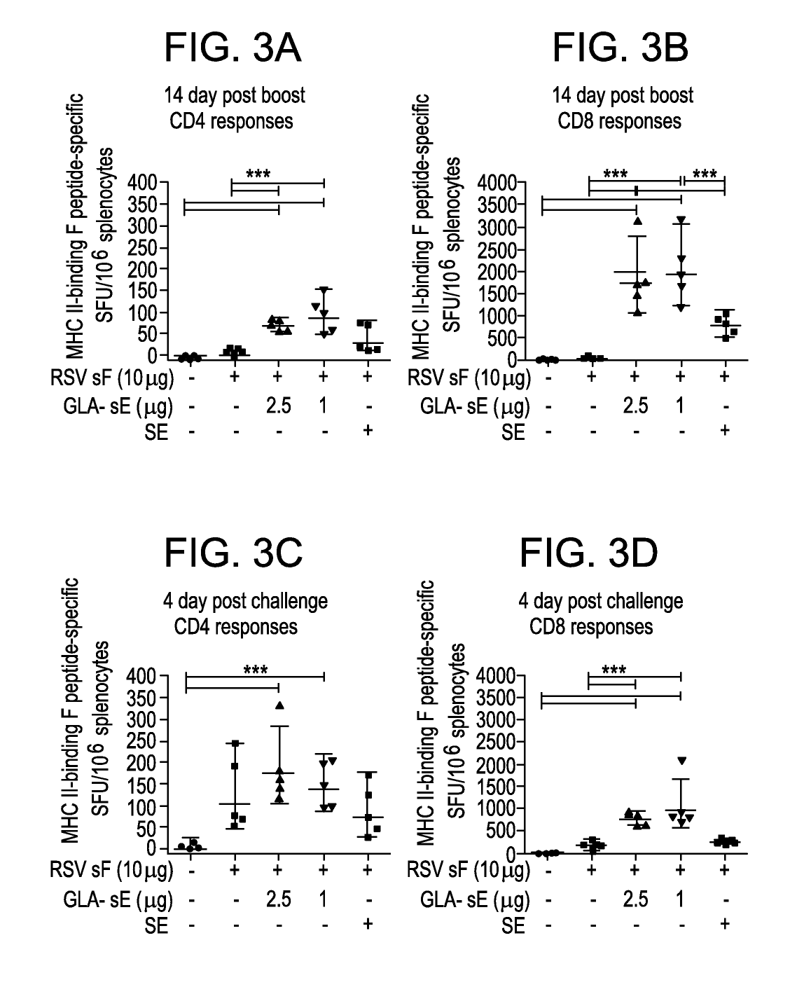

Post challenge F-specific T cells primed by RSV sF+GLA-SE vaccines were easily detected at all RSV sF doses that provided protection. However, it was difficult to detect significant numbers of F-specific T cells before RSV challenge in cohorts vaccinated with 0.3 μg RSV sF or less (data not shown). To evaluate T cell induction in the absence and presence of RSV challenge, mice were vaccinated with 10 μg RSV sF adjuvanted with GLA-SE (2.5 μg or 1 μg in 2% SE) at day 0 and day 14, with one cohort evaluated at 14 days post the second vaccine dose and another evaluated at 4 days post the live RSV challenge. At 14 days post boost, F-specific CD4 and CD8 T cell numbers were significantly enhanced in both sF+GLA-SE groups (mean 49-150 SFU/10⁶ for CD4 responses and 1069-3172 SFU/10⁶ for CD8 responses) compared to either the PBS or the unadjuvanted sF group (FIG. 3A-B). F-specific CD8 T cell numbers in both sF+GLA-SE groups were also significantly greater than those observed in the sF+SE group. Post RSV challenge, F-specific CD4 and CD8 T cell numbers were significantly enhanced in both sF+GLA-SE groups compared to the PBS group (FIG. 3C-D). F-specific CD8 T cell numbers in both sF+GLA-SE groups were also significantly greater than those observed in the unadjuvanted sF group. Interestingly, the absolute numbers of F-specific splenic CD8 appeared lower in the post challenge cohort compared to the pre-challenge cohort, potentially indicating a relocalization of these cells to the site of viral challenge. Together, these data indicate that immunization with RSV sF protein adjuvanted with GLA-SE elicits a systemic F-specific CD4 and CD8 T-cell response that exists prior to any exposure to live RSV.

4. CD8 T Cell Responses Induced by Vaccination with GLA-SE Adjuvanted RSV sF Vaccines are Recruited to the Lungs Following RSV Challenge

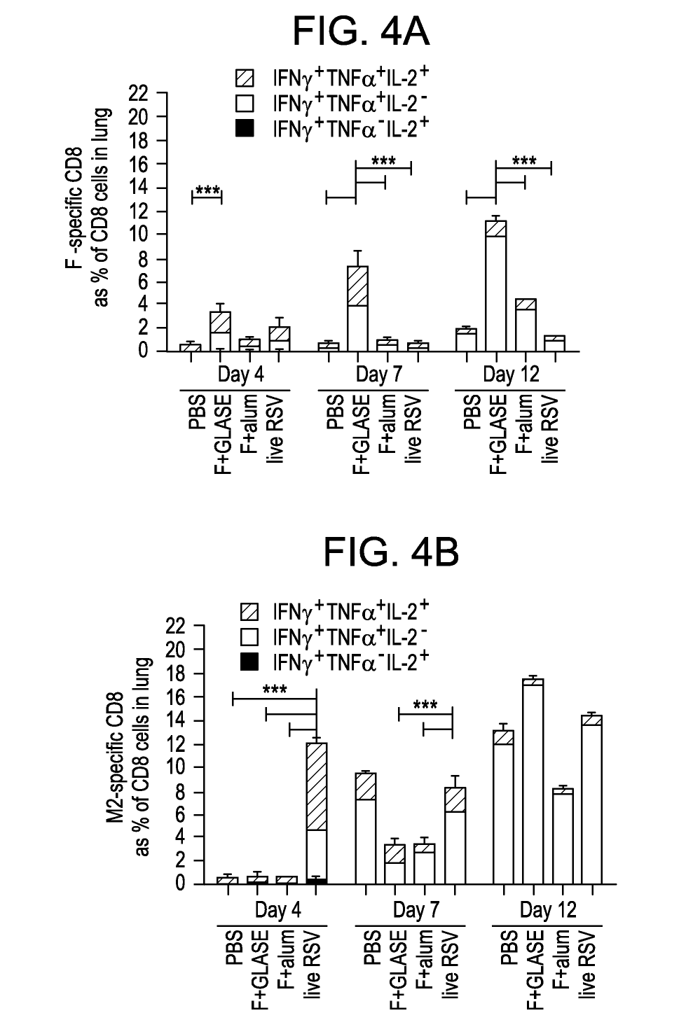

Systemic F-specific CD8 T-cells generated by intramuscular vaccination with GLA-SE adjuvanted RSV sF were evaluated for their ability to traffic to the lungs following RSV challenge. Mice vaccinated with adjuvanted RSV sF (0.3 g) and challenged with RSV A2 had lung lymphocytes (n=3 per group and per timepoint) harvested at days 4, 7 or 12 post challenge for flow cytometric analysis. Mice vaccinated with RSV sF+GLA-SE had 3.39% F-specific CD8 T cells in the lungs by 4 days post challenge, a significant difference from the 0.48% F-specific CD8 T cells observed in the lungs of PBS immunized mice (FIG. 4A). These F-specific CD8 T cells were predominately triple positive for IFNγ, TNF, and IL-2 (mean 1.75%) or double positive for IFNγ and TNF (mean 1.5%). In comparison, the sF+alum vaccine group had only 1.0% F-specific CD8 of any function in the lungs at this timepoint. By day 7 post challenge, mice vaccinated with RSV sF+GLA-SE had 7.28% F-specific CD8 T cells in the lungs, a significant difference from the 0.44% F-specific CD8 T cells observed in PBS immunized mice, the 0.87% observed in sF+alum immunized mice, or the 0.76% observed in live RSV immunized mice (FIG. 4A). The differences in lung-localized F-specific CD8 T cells in these groups at day 12 post challenge were similarly significant, although by this time point the predominant T cell populations were double positive for IFNγ and TNF, having lost IL-2 production. As T cells that lack IL-2 are less proliferative, these cells could represent one of the first steps of the contraction phase. These data indicate a more rapid recruitment of polyfunctional F-specific T cells to the lung following RSV challenge in the RSV sF+GLA-SE group compared to either control PBS immunized animals, RSV sF+alum immunized animals, or even live RSV infected animals.

While local lung F-specific responses are weak in animals with a primary RSV infection, immunodominant M2-specific responses in the lung developed rapidly following secondary infection (FIG. 4B). Over 12% of the total lung CD8 population were M2-specific by 4 days following RSV reinfection, a significantly higher number than observed in the other groups (<1%). These M2-specific CD8 T cells were primarily triple positive CD8 T cells. The number of M2-specific CD8 T cells in the live RSV group did not change significantly over time, but double positive CD8 T cells became predominant. By days 7-12 following RSV challenge the number of M2-specific CD8 T cells had increased in the PBS, RSV sF+GLA-SE and RSV sF+alum immunized groups, indicating a rapid induction of CD8 T cells to this immunodominant epitope in BALB/c mice upon RSV challenge, even in the absence of viral replication.

5. GLA-SE Adjuvanted RSV sF Vaccines Avoid Lung Th2 Responses and Aggravated Lung Histopathology Following RSV Challenge in BALB/c Mice.

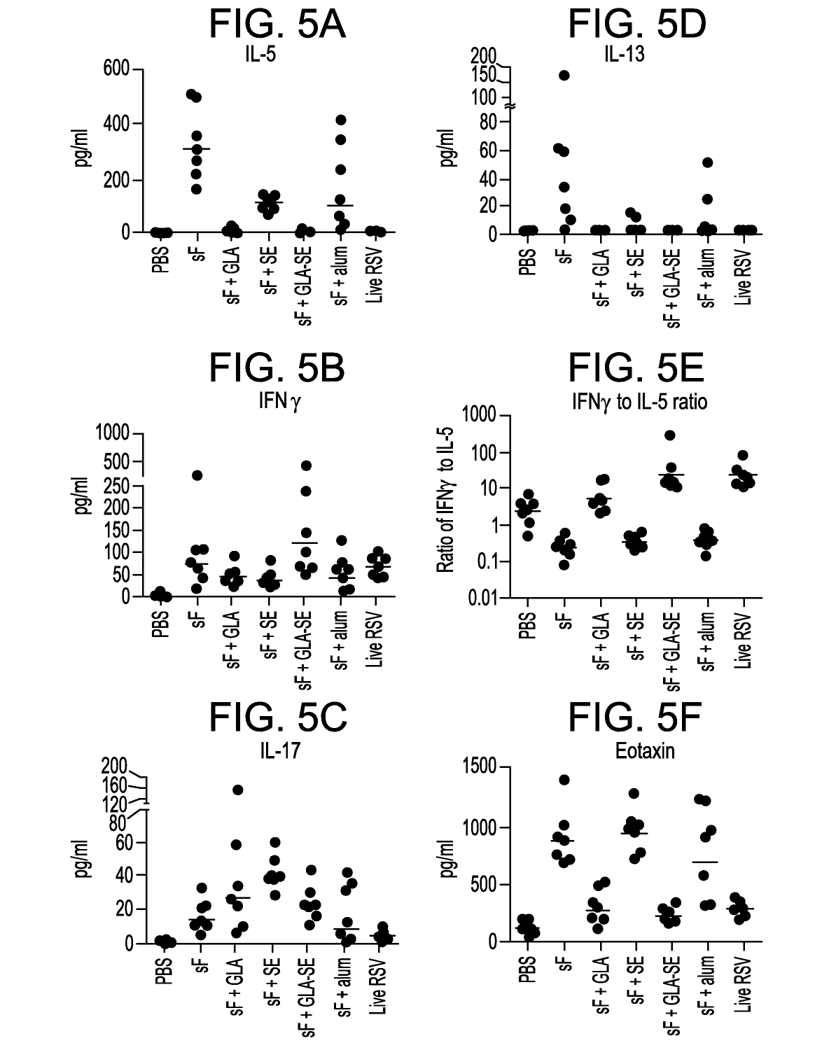

Th2-type responses to RSV challenge in the BALB/c lung, particularly those characterized by IL-13 production, have been reported to correlate with eosinophilic infiltration in the lungs and aggravated histopathology in naive animals (Johnson T R, et al. (2008) Pulmonary eosinophilia requires interleukin-5, eotaxin-1, and CD4+ T cells in mice immunized with respiratory syncytial virus G glycoprotein. J Leukoc Biol 84: 748-759). To determine if any of the adjuvanted RSV sF vaccines induced biased cytokine responses in the lungs of immunized mice, we measured IL-5, IL-13, IFNγ, IL-17, and eotaxin in individual lung homogenates harvested 4 days post RSV challenge. These cytokine readouts provide a snapshot of the cytokines made by any immune cells recruited to the lung, including macrophage, eosinophils, B cells, and T cells. IL-5 and IL-13 were detected only in the lungs of mice immunized with unadjuvanted sF, SE adjuvanted sF, or alum adjuvanted sF, while IFNγ was detected in most of the groups. The ratio of IFNγ to IL-5 was used to express the Th1/Th2 character, with a ratio >1.0 indicating a more Th1-type response. PBS-immunized animals had low levels of all tested cytokines as expected at this early time point following RSV challenge (FIGS. 5A-F). Th1-responses were observed in the live RSV group (mean IFNγ to IL-5 ratio: 29.2), the GLA adjuvanted sF group (mean ratio: 7.8) and the GLA-SE adjuvanted sF group (mean ratio: 59.3). However, a Th2-type response was observed for the unadjuvanted sF group (mean ratio: 0.3), the SE adjuvanted sF groups (mean ratio 0.4), and the alum adjuvanted group (mean ratio: 0.5) (FIGS. 5A-F). Though Th17 cells have been associated both with enhanced inflammation and with enhanced protection in various preclinical lung infection models, only low levels of IL-17 were detected in immunized mice, and these did not vary significantly with the use of adjuvants (FIGS. 5A-F). Eotaxin (CCL11), a chemokine associated with eosinophilic infiltrate (Matthews S P, et al. (2005) Role of CCL11 in eosinophilic lung disease during respiratory syncytial virus infection. J Virol 79: 2050-2057), was at baseline levels of 135 μg/mL in the PBS group and 297 μg/mL in the live RSV group (FIGS. 5A-F). Elevated pulmonary eotaxin levels were observed in groups with Th2-type immune responses including the unadjuvanted sF group (mean: 911 μg/mL), the SE adjuvanted sF group (mean: 965 μg/mL), and the alum adjuvanted sF group (mean: 796 μg/mL). In contrast, pulmonary eotaxin levels in groups with Th1-type immune responses were at baseline, with the GLA-SE adjuvanted sF group at 240 μg/mL.

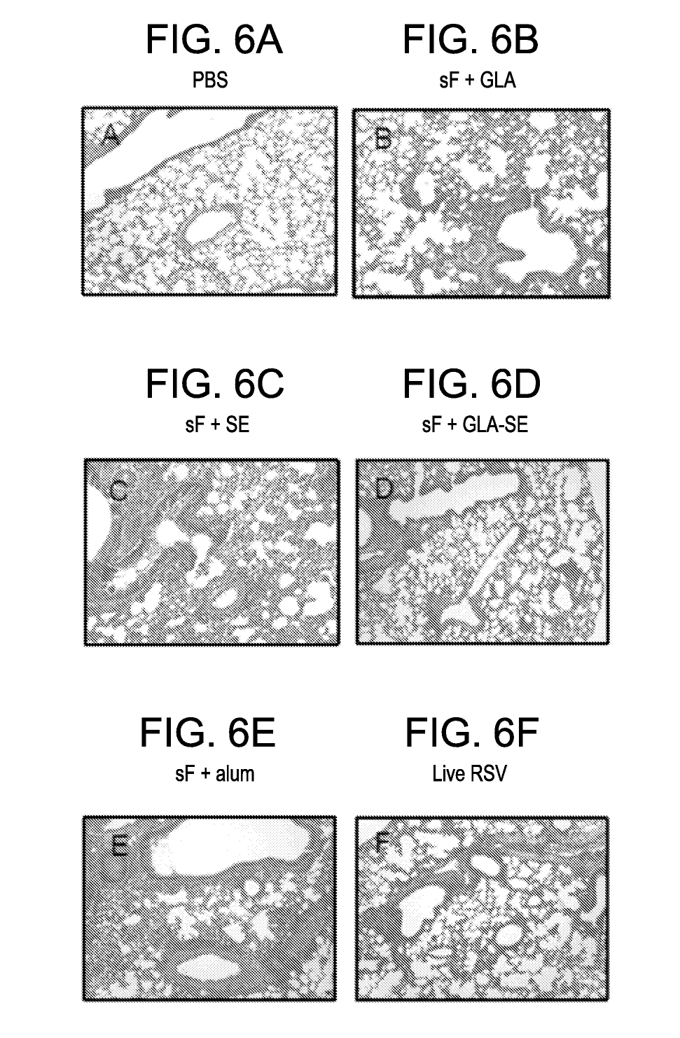

To further evaluate eosinophilic infiltration, lung sections from each vaccine group were scored for histopathological lesions following RSV challenge. Few pulmonary lesions were detected in the lungs of animals experiencing a primary infection with RSV, while a low level of alveolitis and perivascular infiltration was noted in those with a secondary RSV infection (FIGS. 6A-F). Animals that received GLA-SE adjuvanted RSV sF formulations had low pulmonary inflammation scores, similar to mice experiencing a second RSV infection. However, animals that received SE adjuvanted RSV sF, alum adjuvanted RSV sF or unadjuvanted RSV sF had increased pulmonary lesion scores. These data together demonstrate that the observed systemic Th1-biased immune response achieved by immunization with GLA-SE adjuvanted RSV sF corresponds with a lung Th1-biased immune response, baseline lung eotaxin levels, and low lung pulmonary inflammation following RSV challenge in naive BALB/c mice compared to other tested formulations.

6. Adjuvanted RSV sF Subunit Vaccines Confer Complete Protection from RSV Challenge and Induce Both RSV Neutralizing Titers and Th1-Biased Cell-Mediated Immunity in Naive Cotton Rats

Cotton rats are a well established model for RSV studies and are often used in the preclinical evaluation of potential RSV vaccine candidates. To confirm the immune profile of GLA-SE adjuvanted RSV sF vaccine in a second RSV challenge model, individual cotton rats were administered the same RSV sF subunit vaccines at similar doses used for mice. RSV sF at 0.3 μg without adjuvant or adjuvanted with GLA, SE, GLA-SE, or alum was given intramuscularly at days 0 and 22. One group of cotton rats was immunized with GLA-SE alone as a negative control, while another group was given one intranasal dose of 1×10⁶ pfu of live RSV A2 virus at day 0 as a positive control.

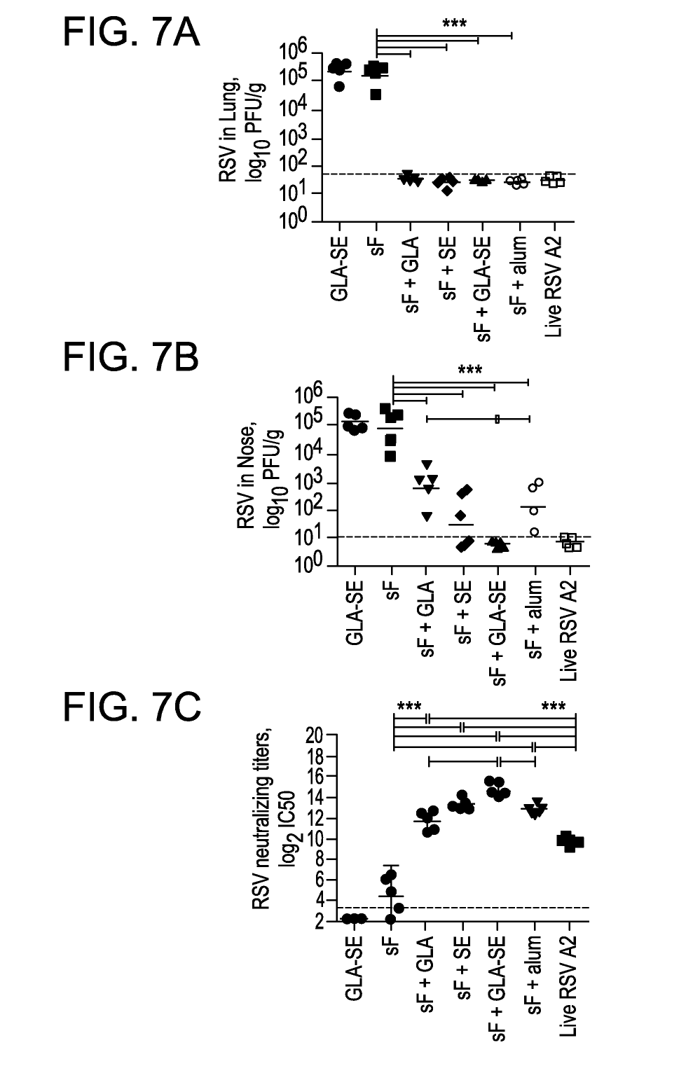

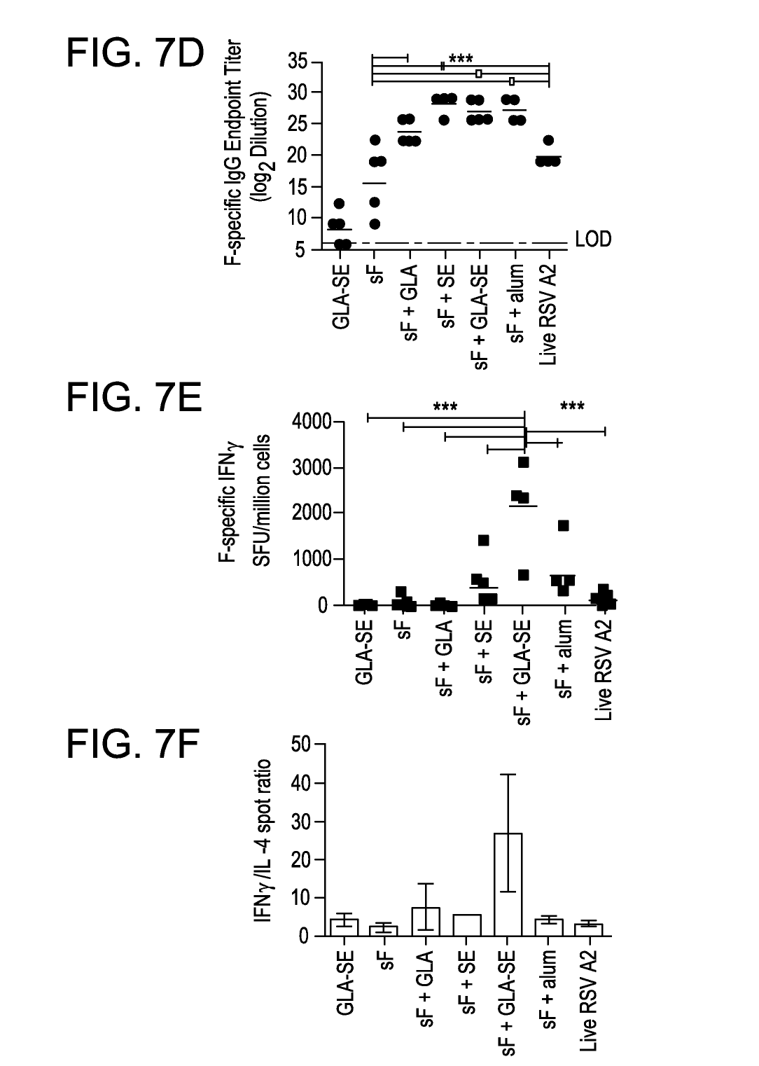

Following RSV challenge, all cotton rat cohorts that received adjuvanted RSV sF vaccines were fully protected in the lung equivalent to the live RSV group, with a mean RSV titer <2 log₁₀ pfu/gram, a 1000-fold reduction in RSV titers compared to the placebo group (5.5 log₁₀ pfu/gram) (FIG. 7A). In contrast to what was observed in mice, immunization with unadjuvanted RSV sF did not protect cotton rats from RSV challenge. The mean viral titer in the lungs of these animals (5.4 log₁₀ pfu/gram) was similar to that of placebo controls. Adjuvanted RSV sF vaccines were also able to protect the upper respiratory tract (nose) of cotton rats from RSV challenge. The cohort vaccinated with sF+GLA-SE showed complete protection in the nose equivalent to that of the live RSV group, both with a mean RSV titer <1 log₁₀ pfu/gram, a 1000-fold reduction in RSV titers compared to the placebo group (5.1 log₁₀ pfu/gram) (FIG. 7B). Partial protection of the upper respiratory tract was observed in groups that received sF+GLA (mean 2.7 log₁₀ pfu/gram), sF+SE (mean 1.4 log₁₀ pfu/gram), or sF+alum (mean 2.1 log₁₀ pfu/gram), though these decreases were all significant compared to the unadjuvanted RSV sF vaccine group (4.9 log₁₀ pfu/gram) or the placebo group.

Cotton rats in the GLA-SE adjuvanted RSV sF vaccine group generated the highest RSV neutralizing titers at day 42, with a mean of 14.7 log₂ (FIG. 7C). This was significantly higher than any other vaccine formulation with the exception of SE adjuvanted RSV sF. High neutralizing titers were also observed for the GLA adjuvanted RSV sF vaccine group (mean 11.7 log₂), SE adjuvanted RSV sF vaccine group (mean 13.3 log₂) and alum adjuvanted RSV sF vaccine group (mean 12.9 log₂). These titers were significantly greater than those achieved by an intranasal infection with live RSV A2 virus (mean 9.7 log₂) or by intramuscular immunization with unadjuvanted RSV sF (mean 4.3 log₂), indicating the superiority of adjuvanted RSV sF in inducing high titer serum RSV neutralizing antibodies. Total F-specific IgG ELISA titers at day 42 post initial vaccination were also higher in the SE, GLA-SE, or alum adjuvanted RSV sF groups than in either the unadjuvanted RSV sF group or the live RSV group (FIG. 7D).

T cell responses in the cotton rat were measured by IFNγ ELISPOT following restimulation with whole RSV sF protein. The strongest F-specific IFNγ ELISPOT response was detected in the GLA-SE adjuvanted RSV sF group (mean: 2626 SFU/million cells), a 45-fold increase over unadjuvanted RSV sF (mean: 58 SFU/million) and a significantly stronger response than seen in any other vaccine cohort (FIG. 7E). Though detectable, sF-specific IFNγ responses were not significantly enhanced by GLA (mean: ˜7 spots/million), SE (mean: ˜642) or alum (mean: 1246) compared to the unadjuvanted RSV sF group. The live RSV infected group generated a relatively low splenic sF-specific IFNγ ELISPOT response of 196 spots. These results were similar to that observed in the BALB/c mouse model.

The ratio of IFNγ to IL-4 specific responses as measured by ELISPOT was used to determine the Th1 bias of the cellular immune response in the cotton rat. The IFNγ:IL-4 ratio generated for each group showed that GLA-SE adjuvanted RSV sF generated the most Th1-biased cellular response (ratio: 26.9), while the others hovered between 1 and 10 (FIG. 7F). This Th1 bias in the cotton rat is similar to that seen in the BALB/c mouse.

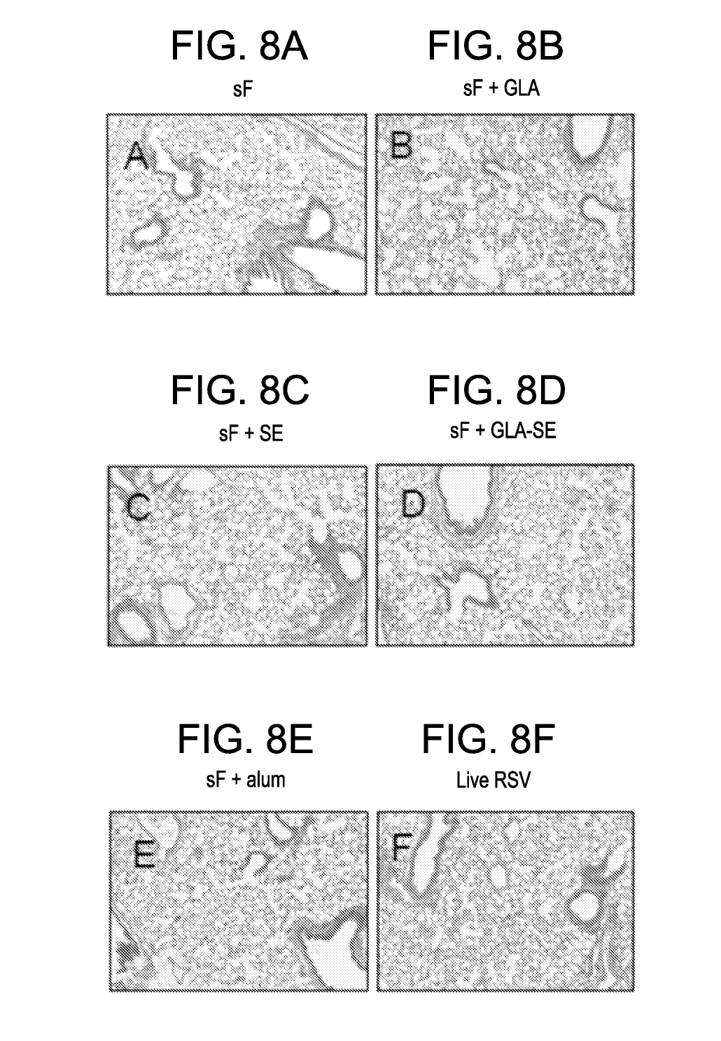

Eosinophilic infiltration and other histopathological lung changes associated with RSV lung pathology were evaluated and scored in cotton rat lung sections collected from all animals at Day 4 post RSV challenge as described for the mouse studies (FIG. 8). No histopathology significantly more severe than seen in the live RSV infected group following a secondary RSV infection was observed in any vaccinated group of cotton rats.

Discussion:

This study demonstrates that intramuscularly administered GLA-SE-adjuvanted vaccines containing purified RSV sF protein are highly immunogenic, generating both high neutralizing titers and a robust Th1-biased cellular response characterized by polyfunctional CD8+ T cells, while fully protecting BALB/c mice and cotton rat from RSV challenge without any indication of immunopathology following RSV infection. In contrast, alum- or SE-adjuvanted RSV sF induced a protective response characterized by high neutralizing titers but a weak and Th2-biased cellular response associated with indicators of lung inflammation, and unadjuvanted RSV sF provided only partial RSV protection to the BALB/c mouse. The study confirms that recombinant RSV sF is likely post-fusion and that in mice GLA-SE adjuvanted RSV sF induces robust cross-neutralizing antibodies to clinical RSV A and B isolates (data not shown).

### Example 2a

### Immunogenicity of RSV-sF in 1×RSV Seropositive BALB/c Mice

This study evaluated the dose response of RSV sF glycoprotein given with or without adjuvant for the ability to boost and maintain RSV specific immune responses in RSV-seropositive BALB/c mice. The goals of this study were to: (1) determine the dose of RSV sF sufficient to boost immune responses in RSV seropositive BALB/c mice following a single vaccine administration; (2) evaluate GLA-SE adjuvant in RSV sF vaccine in boosting RSV immune responses following natural RSV infection; and (3) determine the longevity of boosted F-specific immune responses induced by RSV sF vaccines.

RSV-sF (SEQ ID NO:7) was generated by deletion of the 50 amino acid C-terminal transmembrane domain of the RSV-F human strain A2 protein (i.e., amino acids 525-574) of RSV-sF human strain A2 (SEQ ID NO: 2). Mice were made seropositive by a dose of live RSV virus given intranasally once prior to the initiation of the vaccine study. RSV sF protein was produced from stably transfected Chinese hamster ovary (CHO) cells, immunoaffinity purified, and administered to female BALB/c mice once intramuscularly (Day 0) at 0.4 μg, 2 μg, or 10 μg, either unadjuvanted or adjuvanted with Glucopyranosyl lipid A in a stable emulsion (GLA-SE). Serological anti-F antibody responses and RSV neutralizing antibody responses were measured at Day 0 (baseline) and every 2 weeks for 10 weeks following vaccination. F-specific CD4 and CD8 T-cell responses were measured at 10 days post vaccination in a representative subset of animals (n=3/group) and again following an RSV challenge 10 weeks following vaccination. Local lung-specific immunity post RSV challenge was demonstrated by the presence of antibodies and cytokines.

This study showed that RSV sF administered with or without adjuvant boosted humoral immune responses to RSV in an antigen dose-dependent manner, while RSV sF adjuvanted with GLA-SE also boosted CD8-specific immune responses in an antigen dose-dependent manner. Additionally, this study showed that these boosted responses were maintained for at least 10 weeks following immunization. This study thus indicates that RSV sF+GLA-SE boosted both a humoral and a cellular immune response in mice experimentally infected with RSV before vaccination providing evidence that RSV-sF is a strong candidate vaccine for boosting broad RSV immune responses even in RSV seropositive individuals. A soluble F (sF) protein construct (SEQ ID NO:7) lacking the transmembrane domain of F of RSV human strain A2 (SEQ ID NO: 2) was engineered and expressed from a stable clonal Chinese hamster ovary (CHO) cell line to generate antigenically intact highly purified proteins using immunoaffinity purification.

A widely used model for RSV vaccine evaluations are BALB/c mice, one of the more RSV permissive mouse strains. Reagents are available for the BALB/c mouse model that allows for in depth analysis of immune responses believed to correlate with effective RSV clearance (Connors et al, Resistance to respiratory syncytial virus (RSV) challenge induced by infection with a vaccinia virus recombinant expressing the RSV M2 protein (Vac-M2) is mediated by CD8+ T cells, while that induced by Vac-F or Vac-G recombinants is mediated by antibodies. J Virol. 1992; 66:1277-81). Cross-neutralizing antibodies to RSV (which block both RSV A and RSV B strain infections in tissue culture) are generated in mice, and both mouse as well as human sera contain cross-neutralizing RSV antibodies following RSV infection. BALB/c mice, like humans, are capable of mounting a CD8+ T-cell response to RSV-F glycoprotein which can clear residual infected cells and limit disease (Olson and Varga, Pulmonary immunity and immunopathology: lessons from respiratory syncytial virus. Expert Rev. Vaccines 2008; 7(8):1239-55). These F-specific CD8 T cells can be detected in BALB/c mice against the immunodominant epitope of F glycoprotein, KYKNAVTEL (SEQ ID NO: 12) (Olson and Varga, Pulmonary immunity and immunopathology: lessons from respiratory syncytial virus. Expert Rev. Vaccines 2008; 7(8):1239-55). CD4+ T-cell responses produce cytokines which influence the generation of both neutralizing antibodies and CD8+ T cells, with Th1-type cytokines such as IFNγ being associated with a more effective cellular antiviral response than Th2-type cytokines such as IL-4, IL-5, and IL-13. Th1 responses can be measured directly in the form of cytokines produced at local sites of virus infection or from antigen-restimulated splenic cultures, as well as indirectly by antibody isotypes, with mouse IgG2a isotypes associated with more Th1-type responses. Preclinical animal evaluations in BALB/c mice are designed to select a vaccine formulation that will be sufficiently immunogenic to boost RSV-specific cellular responses in the elderly, avoiding the Th2 bias and overcoming the T-cell defects seen in the elderly compared to the young (Liu et al, Local immune response to respiratory syncytial virus infection is diminished in senescence-accelerated mice. J. Gen. Virol. 2007; 88:2552-8), while at the same time inducing neutralizing antibodies that have been shown to play a key role in the reduction of RSV disease.

Glucopyranosyl Lipid A/Stable Emulsion (GLA-SE) is a combination adjuvant (Immune Design Corporation, Seattle, Wash.) that was demonstrated to enhance the induction of humoral and cellular immune responses to RSV sF in a 2-dose vaccine regimen in naive BALB/c mice. In this study, we determined whether adjuvant is needed in a single-dose RSV sF vaccination regimen to boost immune responses in BALB/c mice experimentally infected with RSV prior to vaccination.

Vaccine formulations evaluated included RSV sF at 0.4 μg, 2 μg, and 10 μg with and without the adjuvant GLA-SE. These were compared to control RSV seronegative animals, seropositive animals given a placebo vaccine, and seropositive animals given a secondary RSV infection as a booster. Immune parameters evaluated include serum antibody responses to RSV sF (total, IgG1/IgG2a, and virus-neutralizing titers), F-specific interferon gamma (IFNγ)-specific CD8 T-cell responses following vaccination, and following recall challenge 10 weeks post vaccination, F-specific Th1/Th2 cytokine-producing CD4 T cells at both these timepoints, and lung cytokine levels and F-specific antibodies at 4 days post recall challenge.

Naïve female BALB/c mice were divided into designated vaccine cohorts of 8-9 mice each and dosed at Day 0. Eight of the 9 groups were inoculated with 10⁶ PFU live RSV A2 virus intranasally 28 days prior to vaccine administration to create RSV seropositive animals. Successful seroconversion was confirmed by F-specific ELISA endpoint titers on Day 0. Groups of 9 mice were inoculated intramuscularly (IM) with the vaccine formulations at Day 0. 3 mice per group were evaluated for cellular immune responses at 10 days post challenge, while the remaining 5-6 animals per group were followed for serum antibody responses through Day 73. Remaining animals were challenged at Day 69 with live RSV A2 virus intranasally to allow evaluation of residual recall cellular immune responses at 4 days post challenge (Day 73).

3 different doses of RSV sF subunit vaccine were evaluated with or without GLA-SE. The doses used were 0.4 μg, 2 μg, and 10 μg per mouse of subunit protein, which covers the range used in naive BALB/c mice and includes the lowest proposed clinical dose of RSV sF glycoprotein (10 μg). GLA-SE in the adjuvanted groups was given at a dose of 5 μg of GLA in 2% SE. Seropositive mice given a booster infection with 10⁶ PFU live RSV A2 virus intranasally at Day 0 served as positive controls, while negative controls included a seropositive group inoculated with PBS as a placebo and a seronegative group inoculated with PBS as placebo.

Serology readouts were made at Days 0, 14, 28, 42, 56, and 73 for each group. Animals were lightly anesthetized with isoflurane and bled intraorbitally. Serum was separated and stored at −20° C. and thawed for testing. Total anti-F IgG were measured at each timepoint, with anti-F IgG1 and anti-F IgG2a ELISA endpoint dilution titers measured at Day 0 and Day 42. RSV neutralization titer was determined by a RSV A2-GFP microneutralization assay. The polyclonal nature of the anti-F IgG response was evaluated on Day 42 by competition ELISA with site-specific monoclonal antibodies to RSV-F. Anti-F IgA endpoint dilution titers were measured at Day 14 for each group.

Systemic cellular immune responses to vaccination were evaluated in representative animals at Day 10 post vaccination. Additional representative animals were recalled with a viral challenge at Day 69 and evaluated for long-term cellular immune responses at Day 73, 4 days post viral challenge. For each of the groups, 3-5 individual splenocyte samples were prepared. CD4 T-cell readouts were assessed by multiplexed cytokine analysis of supernatant levels of a panel of secreted cytokines (including IFNγ, IL-5, IL-10, IL-13, and IL-17) following a 72-hour restimulation period with RSV sF. CD8 T-cell readouts were assessed by 2 methods: ELISPOT counts of IFNγ-secreting cells following a 36-48 hour restimulation period with an F-derived CD8 peptide (KYKNAVTEL aa 85-93) (SEQ ID NO: 12) and intracellular staining and quantification of the percentage of F-specific polyfunctional (IFNγ+ TNFα+IL-2+) CD8 T cells following a 5-hour restimulation period with the F-derived CD8 peptide.

Lung-specific responses to the viral challenge were assessed on individually harvested homogenized lungs taken at Day 73, 4 days post challenge. Cytokine levels (IFNγ, IL-5, IL-10, IL-13, IL-17, eotaxin) in the lung homogenates were measured as biomarkers of the local cellular immune response. F-specific IgA and IgG antibodies in the lung homogenate were measured by ELISA endpoint titers to show that the antibody responses are targeted to the lung. Significance was calculated using GraphPad Prism 1 way ANOVA with Tukey post test and a significance cutoff of p<0.05.

Results

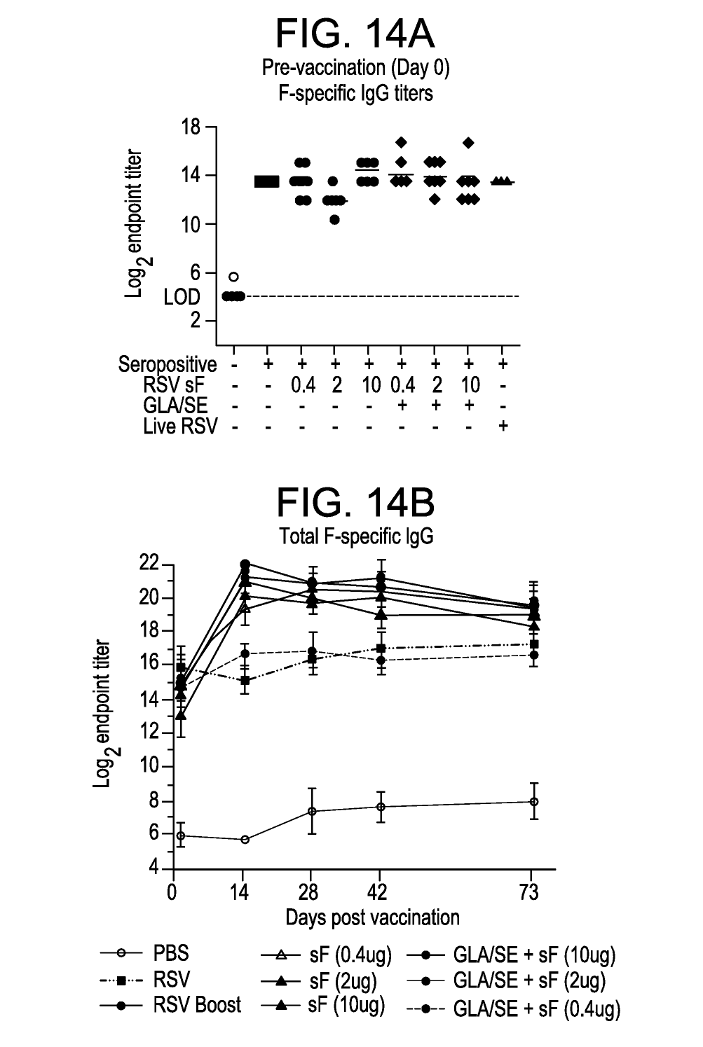

RSV seropositive groups (Groups 2-10) were intranasally infected with a high dose of 10⁶ pfu RSV A2 virus 28 days prior to vaccination. RSV seroconversion in these animals was confirmed by F-specific IgG endpoint ELISA titers at Day 0. All seropositive animals had detectable F-specific IgG at Day 0, with group mean endpoint titers ranging from 12.81-15.36 (average 14.60). In contrast, the control seronegative group had a median titer of 5.64 (FIG. 14). Most of the seropositive animals were also found to have low but detectable neutralizing antibody titers at Day 0, with a mean log₂ 50% plaque reduction titer of 3.07-3.88.

Vaccines were given at Day 0 to all animals. A working stock of 250 μg GLA in 10% SE (generated by diluting GLA-SE [1 mg/mL in 10% SE] with 10% SE) was used to achieve a final vaccine dose of 5 μg GLA in 2% SE in 100 μL.

Boosted F-directed antibody responses were assessed at Day 14, 28, 42, and 73 post vaccination and compared to baseline serological readouts at Day 0 for each vaccine cohort. Total anti-F serum IgG titers at Day 14 indicated that all seropositive animals that received sF vaccines, regardless of antigen dose or its formulation with GLA-SE, quickly responded with a boost in titers (FIG. 14). A 4-fold boost in serum IgG titers is considered significant. The observed boost ranged from 13 to 137-fold at Day 14, the day at which titers were consistently the highest across groups. Seropositive mice that received the PBS vaccine had less than a 4-fold boost in IgG titers, while those that were boosted with live RSV infection had close to a 4-fold boost in IgG titers. Interestingly, similar total F-specific IgG titers were observed between groups that received different doses of RSV sF without adjuvant and groups that received different doses of RSV sF+GLA-SE. The boosted anti-F IgG titers were greater than 15-fold at Day 73 in all these groups, and no dose- or adjuvant-enhanced difference was observed (FIG. 14).

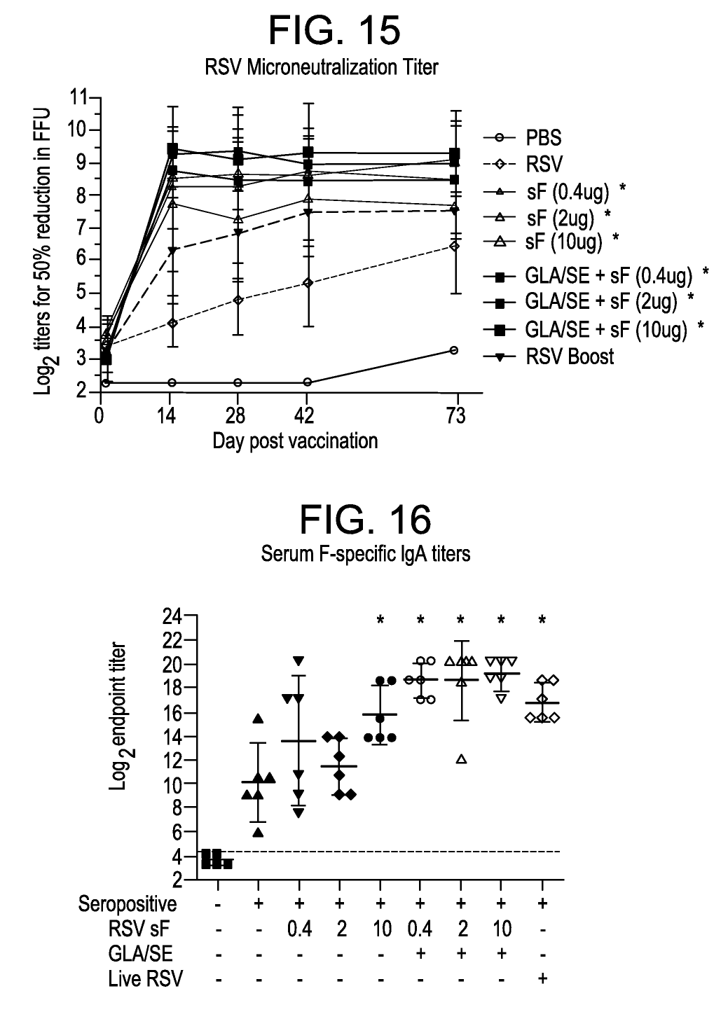

Serum RSV neutralizing titers were also evaluated at multiple time points. The mean log₂ 50% plaque reduction titer for the different groups of RSV seropositive animals at Day 0 ranged from 3.07-3.88 (FIG. 15). By Day 14 post vaccination, animals immunized with either live RSV, RSV sF, or RSV sF+GLA-SE had neutralization titers boosted over their Day 0 values (FIG. 15). A 4-fold boost in titers is considered significant. Seropositive mice given an unadjuvanted RSV sF vaccine demonstrated a 15- to 28-fold boost in RSV neutralization titers, while those administered a GLA-SE adjuvanted RSV sF vaccine demonstrated a 53- to 85-fold boost in neutralization titers. In contrast, seropositive mice that received a PBS vaccine had less than a 2-fold increase in neutralizing titers at Day 14 and those given a second infection with live RSV showed only a 7-fold boost in neutralizing titers. This indicates that RSV sF vaccines boosted neutralizing titers in seropositive mice to a greater degree than re-infection with RSV. The amount of RSV sF (0.4-10 μg) was not important in this induction, as the mean RSV neutralizing titers at Day 14 for each dose group were within 2-fold of each other (8.32, 7.82, and 8.66 for unadjuvanted doses, and 9.32, 8.82, and 9.49 for adjuvanted doses). The inclusion of adjuvant provided only a ˜2-fold enhancement in boosted neutralizing antibodies in RSV seropositive mice in contrast to what is observed in naive mice, where without an appropriate adjuvant very few neutralizing antibodies are induced by RSV sF vaccines. The neutralization titers for each group remained within 80% of the Day 14 values out to Day 73, in some instances increasing over time (FIG. 15). This indicates a persistence of functional humoral immunity for at least 10 weeks post immunization.

Serum IgA is more amenable to measurement than mucosal IgA in live mice and may give an indication of the levels of mucosal IgA. At Day 14 post vaccination seronegative animals had very low F-specific IgA titers that were less than or equal to the limit of detection, but all seropositive animals had detectable F-specific IgA (FIG. 16). Seropositive animals vaccinated with RSV sF (10 μg) or RSV sF at any of the 3 doses+GLA-SE generated significantly higher serum F-specific IgA titers than seropositive animals vaccinated with PBS. Seropositive animals boosted with a second RSV A2 live infection also showed significantly higher serum F-specific IgA titers. This indicates that RSV sF+GLA-SE vaccines can boost serum IgA titers in seropositive animals.

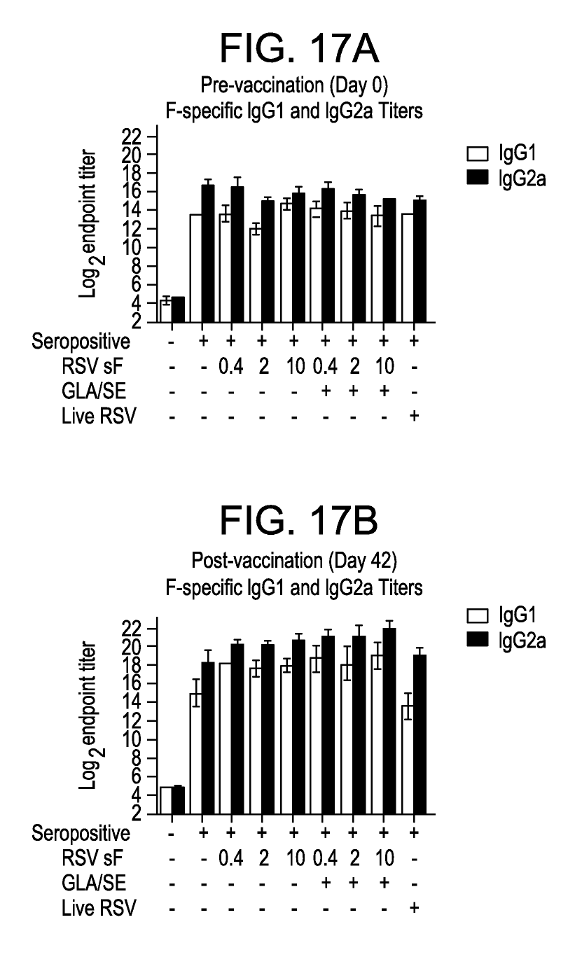

Serum F-specific antibodies at Day 0 and at Day 42 were also evaluated for IgG1 and IgG2a isotypes to determine the T helper type balance of the seropositive animals before and after vaccination. F-specific IgG1 titers (a Th2-type subtype) and F-specific IgG2a (a Th1-type subtype) titers were both present in seropositive animals at Day 28 (FIG. 17). IgG2a titers predominated in seropositive animals prior to vaccination and maintained their dominance post vaccination at Day 42 regardless of vaccine formulation received (FIG. 17). This is in contrast to prior studies in naive animals, where RSV sF vaccines given without adjuvant primarily induced an IgG1 response and inclusion of GLA-SE adjuvant was needed to induce an IgG2a-biased.

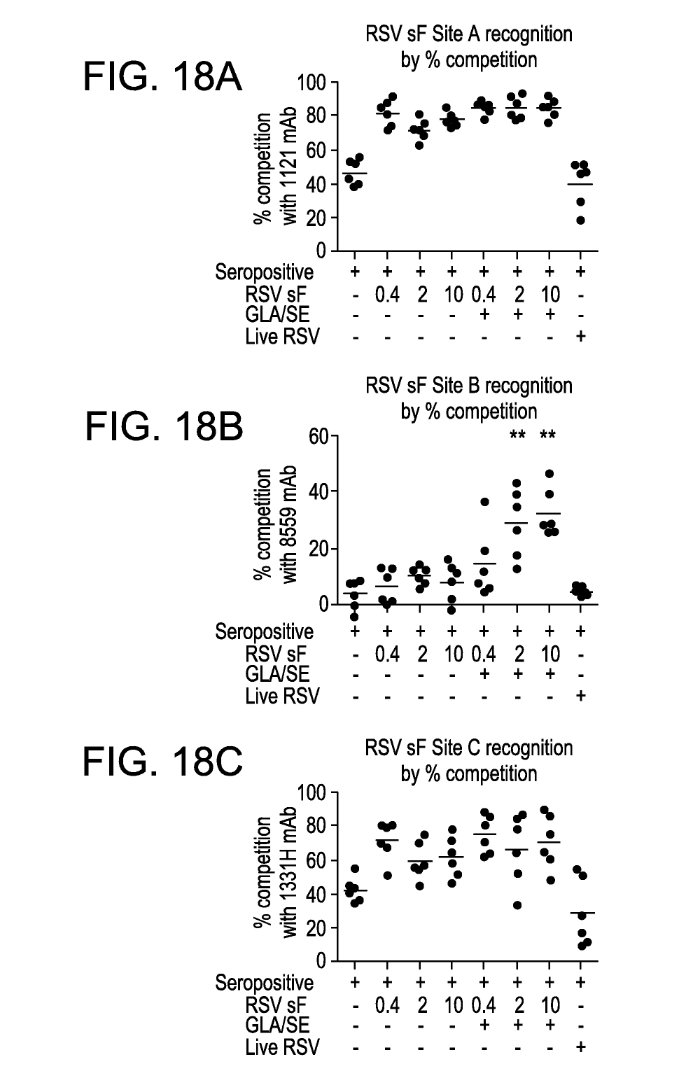

To determine whether RSV sF vaccines boosted polyclonal serum antibodies against the known neutralizing antigenic sites of RSV sF in seropositive mice was also examined, a competition ELISA assay was used to assess the polyclonality of sera following vaccination by measuring their capacity to block binding of site A, B and C-specific mAb to the target epitope on the RSV sF antigen. Sera from all tested groups showed strong competition with Site A and Site C antibodies and detectable competition with Site B antibodies, indicating a polyclonal RSV-F-directed response (FIG. 18). Sera from each of the RSV sF vaccinated groups (±GLA-SE adjuvant) was better at competing for site A and site C binding than sera from seropositive animals boosted with PBS or live RSV, indicating an advantage for RSV sF vaccines over natural infection. Interestingly, competition for site B binding was adjuvant and RSV sF dose dependent. Sera from mice that received GLA-SE adjuvanted RSV sF at the 2 μg or 10 μg dose have significantly enhanced site B competition responses relative to sera from their matched unadjuvanted groups (FIG. 18).

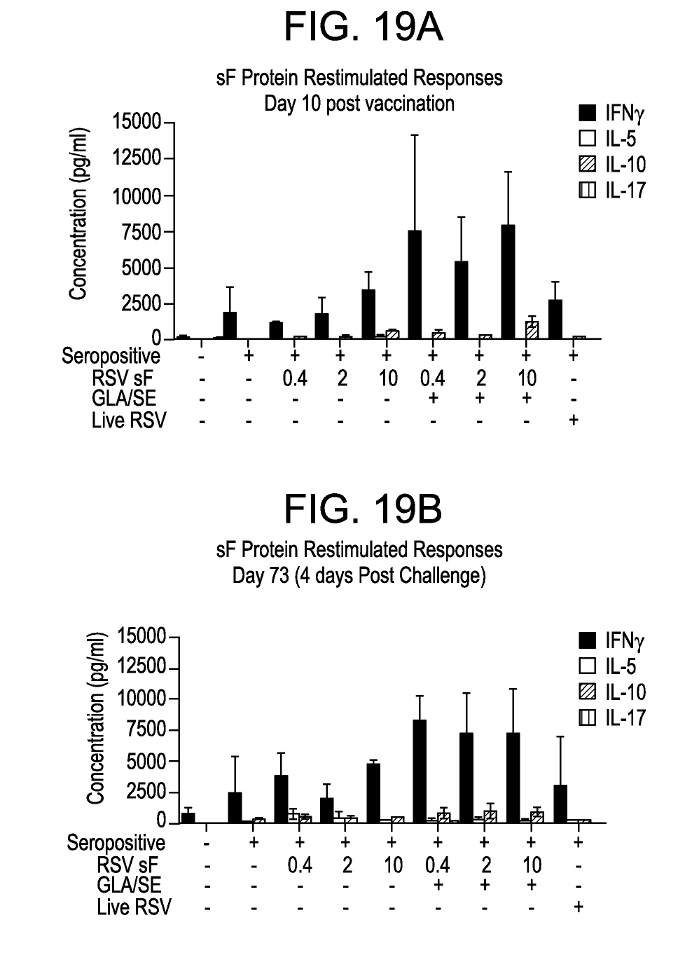

Systemic CD4 T-cell immune responses were evaluated at 2 separate timepoints. At Day 10 post vaccination, splenocytes were harvested from 3 animals in each group and restimulated with RSV sF-protein for 72 hours for measurement of cytokines by Bioplex. While the seronegative group gave no F-specific cytokine responses across the panel tested, F-specific IFNγ (a Th1 cytokine) was detected in all the seropositive groups at Day 10 (FIG. 19). The magnitude of the response appeared greater in the GLA-SE adjuvanted RSV sF group compared to seropositive mice that received unadjuvanted sRSV-F. In comparison to IFNγ, IL-5 (a Th2 cytokine), IL-10 (a Th0 cytokine), and IL-17 (a Th17 cytokine) were detected only at very low levels, indicating that the seropositive animals displayed a Th1-biased response regardless of the vaccine used. CD4 T cell immune responses were also evaluated at Day 73, 4 days post a recall infection with RSV. Splenocytes were harvested from 3-5 animals in each group and cytokine responses were measured by Bioplex. Again, F-specific IFNγ indicative of a strong Th1 response was the predominant cytokine observed, with low levels of the representative Th0, Th2, or Th17 cytokines (FIG. 19). This data is consistent with the F-specific IgG1/IgG2a titers observed in these seropositive mice and is in contrast to what was seen in the naive-mouse, where unadjuvanted RSV sF vaccines induced a Th2-type immune response. These data suggest that in the seropositive mouse model, a Th1-bias set up by the initial RSV infection informs the character of future boosted responses to RSV-F vaccines. This Th1-bias suggests that the model may better represent healthy adults who have prior experience with RSV infection but may not reflect vaccine responses in immunosenescent elderly population who are seropositive for RSV.

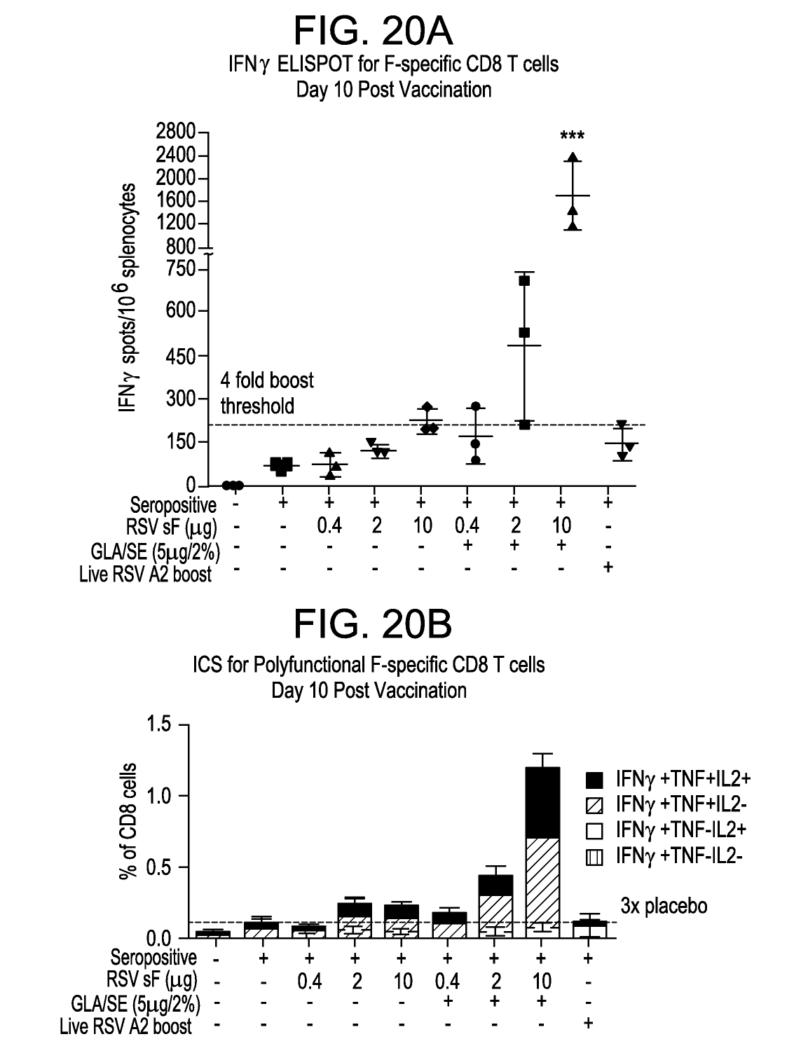

CD8 T-cell immune responses were evaluated in each group of animals at the same 2 timepoints. At Day 10 post vaccination, 3 animals per group were evaluated by IFNγ-ELISPOT with CD8 F peptide restimulation. The placebo group lacked F-specific CD8 responses (0 SFU/million cells), while the seropositive animals had a low detectable CD8 response of 69 SFU/million (FIG. 20). In contrast, groups dosed with unadjuvanted sF had a dose dependent F-specific CD8 IFNγ-response (mean 72-224 SFU/million), while groups dosed with sF+GLA-SE had a dose-dependent F-specific CD8 IFNγ-response of greater magnitudes (mean 171-1699 SFU/million). At the highest adjuvanted dose of RSV sF, the magnitude of the observed CD8 response (1699 SFU/million) was significantly higher than the same dose without adjuvant (224 SFU/million), and much higher than observed in the live RSV boosted group (145 SFU/million) (FIG. 20). This CD8 IFNγ ELISPOT response was the highest measured. All groups were also evaluated by intracellular flow cytometry for polyfunctional CD8 T cell responses characterized by expression of IFNγ, TNFα (an effector cytokine), and IL-2 (a survival cytokine). The presence of polyfunctional CD8 T cells has been correlated with viral protection, suggesting that these cells may be more effective at the clearance of virally infected cells (Betts et al, HIV nonprogressors preferentially maintain highly functional HIV-specific CD8+ T cells. Blood. 2006; 107(12):4781-9). As expected from the IFNγ ELISPOT response, a significant GLA-SE-adjuvanted RSV sF dose-dependent CD8 response was observed. Seropositive mice boosted with adjuvanted RSV sF at 10 μg showed triple-positive polyfunctional anti-F CD8 T cells at a frequency of 0.49% of all CD8 T cells. 0.62% of all CD8 T cells showed dual IFNγ and TNFα F-specific activity. This easily surpassed the threshold levels (0.03-0.06%) based on 3× the mean frequency of F-peptide restimulated responses in the seronegative placebo group (0.01-0.02%) (FIG. 20). Detectable triple (0.13%) and double (0.27%) positive polyfunctional F specific CD8 T cells were also detected in the group dosed with 2 μg RSV sF adjuvanted with GLA-SE (FIG. 20).

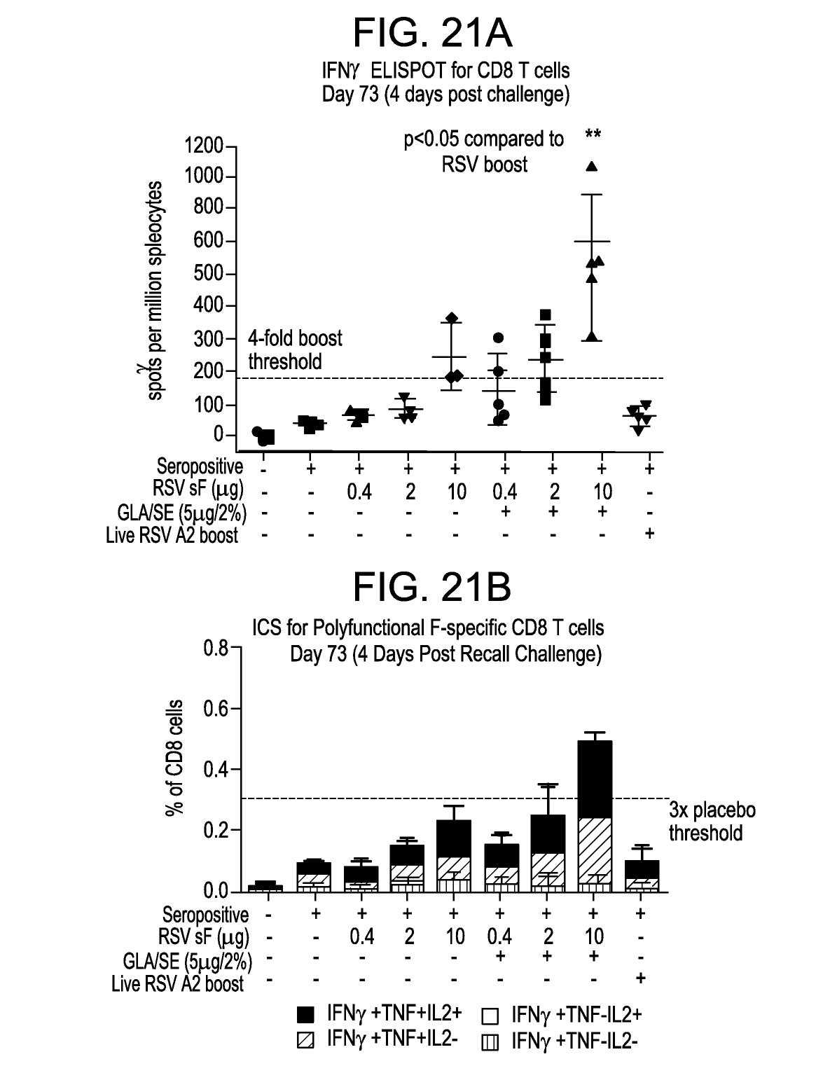

To evaluate the persistence of the CD8 response, 3-5 mice/group were evaluated at Day 73 (4 days post RSV challenge) for recall CD8 T-cell immune responses by both methods. IFNγ ELISPOT detected dose-dependent F-specific CD8 IFNγ-responses (means 142-598 SFU/million) in groups dosed with sF+GLA-SE (FIG. 21). This was more than observed with matched unadjuvanted sF groups, which also showed a dose-dependent CD8 IFNγ response (62-243 SFU/million). In comparison the seronegative control gave no IFNγ response (−4 SFU/million), and lower responses were seen when the seropositive mice were boosted with PBS (35 SFU/million), or live RSV A2 (61 SFU/million). Intracellular flow cytometry detected strong polyfunctional F-specific CD8 T cells in the group that received the highest adjuvanted dose of RSV sF (0.25% triple positive and 0.25% double positive) (FIG. 21). The magnitude of the response was slightly lower than that detected at Day 10 post vaccination, but the data shows that F-specific CD8 responses can be recalled 10 weeks following vaccination.

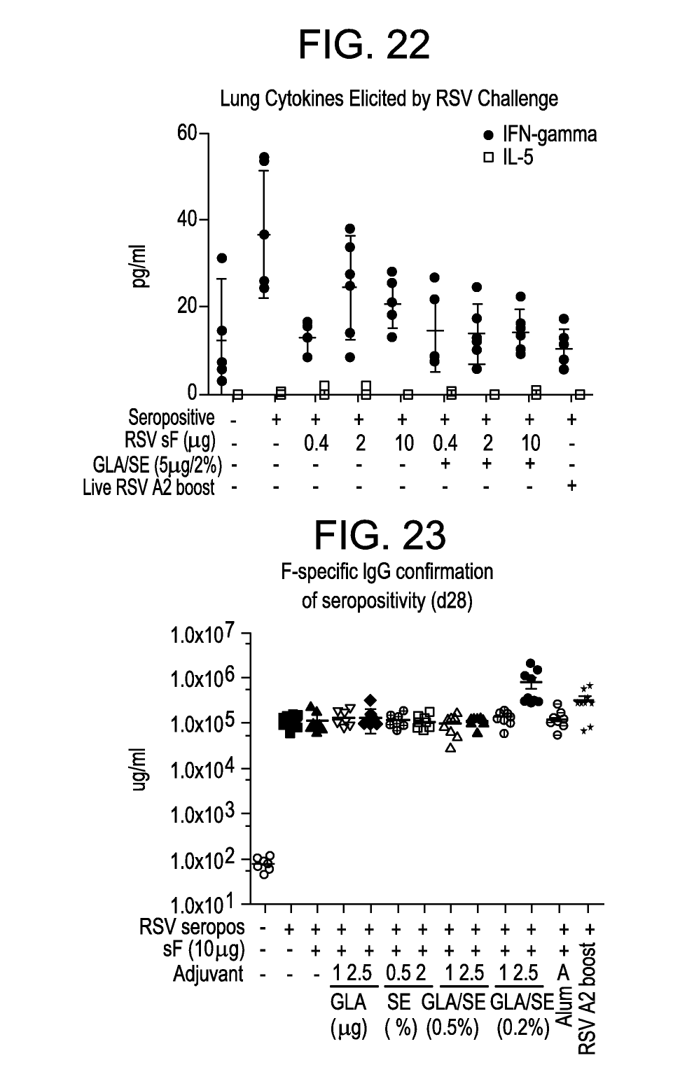

To confirm a persistence of the Th1 character of the immune response in the local lung environment following RSV challenge, levels of cytokines such as IFNγ, IL-5, IL-13, IL-10, IL-17, and eotaxin were evaluated using the Day 73 lung homogenates (FIG. 22). These cytokine readouts provide a snapshot of the cytokines made by any immune cells recruited to the lung, including macrophage, eosinophils, B cells, and CD4 or CD8 T cells. The primary cytokine detected in the lung homogenates from seropositive immunized mice was IFNγ, with very little IL-5, IL-10, IL-13, or IL-17 detected following recall RSV challenge. Eotaxin, a cytokine that can induce the chemotaxis of eosinophils associated with lung immunopathology in naive animals, was expressed in all groups at levels similar to that of the seronegative naive animals mounting a first response to RSV infection. These data indicate that the lung immune response in vaccinated seropositive animals reflects the character of the systemic immune response and remain Th1-biased with a low risk of eosinophilia.

Conclusions

This study found that one inoculation with either unadjuvanted or GLA-SE adjuvanted RSV sF at antigen doses from 0.4-10 μg can significantly boost serological readouts of immunity in RSV seropositive BALB/c mice. Neutralizing antibodies were detected by a RSV microneutralization assay and persist for 10 weeks post vaccination. Cellular CD8 immunity to RSV sF was observed to be antigen dose-dependent and to require GLA-SE adjuvant, with significantly boosted numbers of polyfunctional CD8 T cells in seropositive mice at the highest (10 μg) dose of RSV sF+GLA-SE. This was observed both within 10 days of vaccination and following a recall challenge 10 weeks after vaccination. The Th1-biasing adjuvant GLA-SE was observe to play an important role in enhancing CD8 T cells, serum RSV-F site B-specific antibodies, and serum F-specific IgA titers in this seropositive model. No advantage of adjuvant was seen in boosting serum neutralizing titers or serum F-specific IgG in this seropositive model. F-specific serum antibodies, F-specific CD4 T cell IFNγ responses, and lung cytokine levels evaluations indicated that this seropositive mouse model was Th1-biased by the initial RSV infection, suggesting that it may model RSV vaccination response in RSV seropositive healthy adults. In a Th2-biased RSV seropositive host such as elderly humans, GLA-SE may offer additional advantages by switching the Th2 helper response to a more Th1-like response as observed in naïve mice.

A second study was run in 1× seropositive BALB/c mice to confirm the observations of boosted neutralizing antibodies and enhanced cellular immunity in seropositive mice given the 10 μg dose of RSV sF+GLA-SE. In this study, the aim was to 1) repeat the observations seen with the 10 μg dose of RSV sF alone, 2) compare this response to that achieved with a 10 μg dose of RSV sF given only with GLA (1 or 2.5 μg), 3) compare this response to that achieved with a 10 μg dose of RSV sF given only with SE (0.5 or 2%), 4) compare this response to that achieved with a 10 μg dose of RSV sF given with a lower dose of GLA-SE (1 or 2.5 μg+0.5% SE or 1 or 2.5 μg+2% SE), and 5) compare this response to that achieved with a 10 μg dose of RSV sF given with alum.

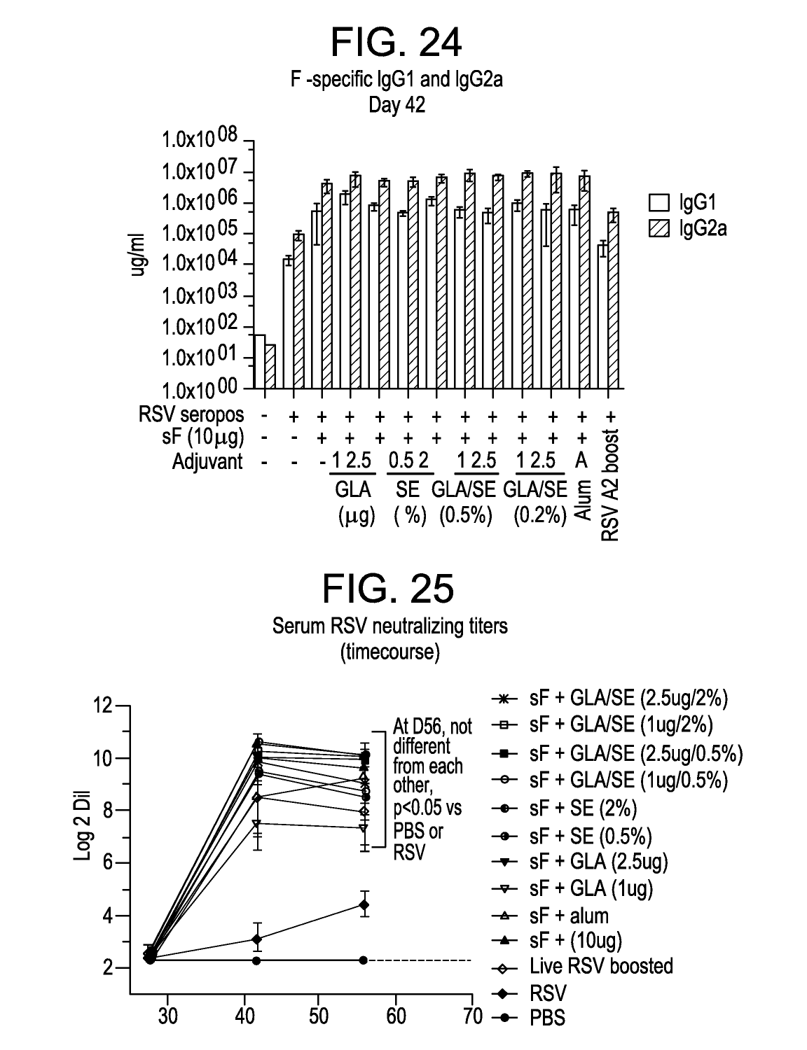

Mice were divided into 13 groups of 9 animals each, with 12 groups (all but the control) made seropositive with a single intranasal infection with a high dose of 10⁶ pfu RSV A2 virus 28 days prior to initial vaccination. RSV seroconversion in these animals was confirmed by serum F-specific IgG endpoint ELISA titers at day of vaccination (FIG. 23). Animals were vaccinated as before, intramuscularly with 100 μl of PBS or formulated RSV sF vaccines. Serum F-specific IgG1 and IgG2a were evaluated at 2 weeks post vaccination. Though the RSV sF vaccines boosted IgG1 and IgG2a titers above that seen in PBS vaccinated animals and above that achieved by a second infection with RSV A2, all seropositive groups had higher IgG2a levels than IgG1 levels regardless of the vaccine given indicating an original Th1 bias (FIG. 24). Serum RSV neutralizing titers were evaluated at 2 weeks, 4 weeks, and 6 weeks post vaccination. Groups that received 10 μg RSV sF, regardless of adjuvant, had neutralizing titers that were boosted significantly over those of the PBS vaccinated 1× seropositive control group and were undistinguishable from each other (FIG. 25).

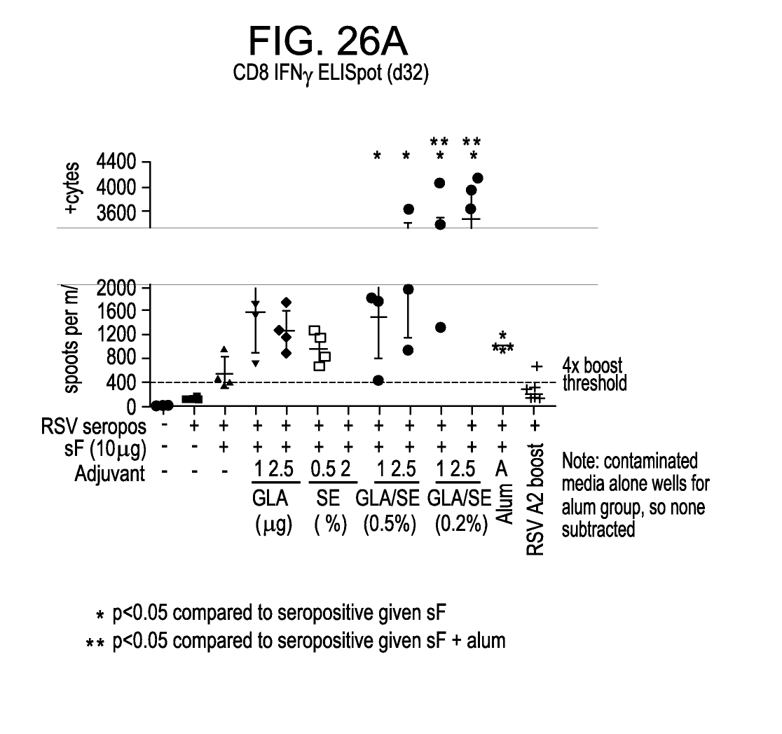

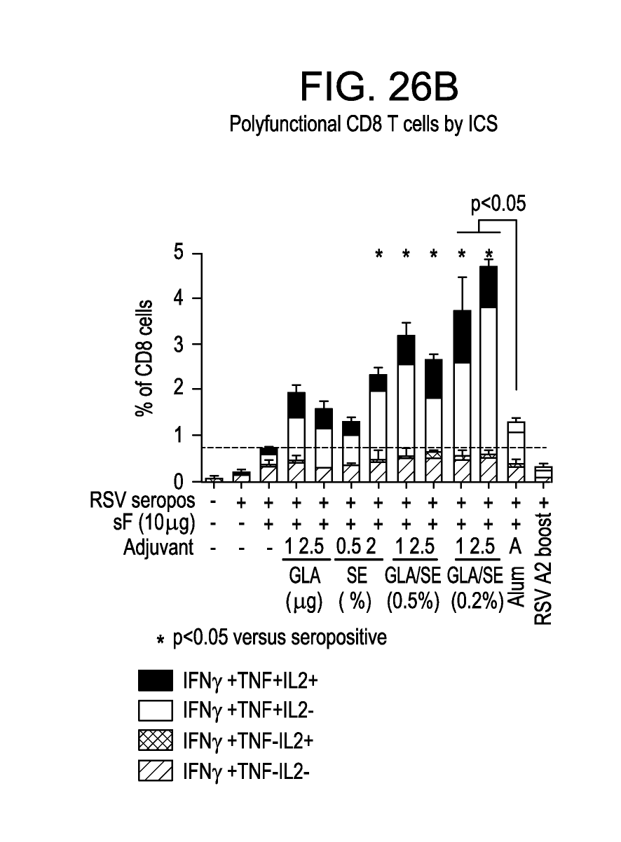

While the choice of adjuvant did not affect the neutralizing antibody response in RSV sF vaccinated 1× seropositive BALB/c mice, it did affect the cellular response achieved. Splenocytes from 3-4 representative animals per group were harvested at 10 days post vaccination to evaluate F-specific CD8 T cell responses by both IFNγ ELISPOT and by intracellular cytokine staining (for IFNγ, TNF, and IL-2 producing polyfunctional cells). In the ELISPOT assay, groups that received sF+GLA-SE at either the 1 or 2.5 μg dose in 2% SE had significantly higher responses than those that received either sF alone or sF+alum (FIG. 26A). This significantly higher response to sF+GLA-SE compared to sF or sF+alum was also seen in the intracellular cytokine staining assay (FIG. 26B). In addition, the intracellular cytokine assay detected an improved F-specific CD8 response in groups given RSV sF+2% SE or RSV sF+GLA-SE (1 or 2.5 μg in 0.5% SE) compared to the group given just RSV sF.

This experiment confirmed the ability of RSV sF+GLA-SE to boost neutralizing titers as well as unadjuvanted RSV sF in 1× seropositive BALB/c mice, and additionally showed that RSV sF+GLA-SE is an optimal formulation in comparison to other adjuvanted RSV sF vaccines for boosting F-specific CD8 T cell responses in seropositive animals.

### Example 2b

### RSV-F Subunit Vaccine Adjuvanted with GLA-SE in Highly Seropositive BALB/c Mice

In this example seropositive Balb/c mice were used to evaluate how RSVsF dose affects response and how adjuvant modulates the response. RSV re-infection occurs throughout life and despite relatively high levels of anti-RSV neutralizing antibodies the elderly (>65 yrs old) are more susceptible to serious RSV associated illness than healthy adults upon RSV re-exposure (Mullooly et al.; Vaccine Safety Datalink Adult Working Group Influenza- and RSV-associated hospitalizations among adults. Vaccine. 2007 25(5):846-55, Walsh E E, Peterson D R, Falsey A R. Risk factors for severe respiratory syncytial virus infection in elderly persons. J Infect Dis. 2004 189(2):233-8). An increase in RSV-associated disease severity in the elderly may in part be due to immunosenesence and a shift toward a Th2 bias in this population which may lead to suboptimal clearing of RSV following infection (Cusi M G, Martorelli B, Di Genova G, Terrosi C, Campoccia G, Correale P. Age related changes in T cell mediated immune response and effector memory to Respiratory Syncytial Virus (RSV) in healthy subjects. Immun Ageing. 2010 Oct. 20; 7:14.). Previous clinical trials using RSV F or F+G+M extracted and purified from the virus showed that in general these RSV antigens provided modest boosting of pre-existing RSV antibody titers with or without alum but these studies did not report boosting of RSV CMI responses (Langley J M, Sales V, McGeer A, Guasparini R, Predy G, Meekison W, Li M, Capellan J, Wang E. A dose-ranging study of a subunit Respiratory Syncytial Virus subtype A vaccine with and without aluminum phosphate adjuvantation in adults > or =65 years of age. Vaccine. 2009 27(42):5913-9. Falsey A R, Walsh E E, Capellan J, Gravenstein S, Zambon M, Yau E, Gorse G J, Edelman R, Hayden F G, McElhaney J E, Neuzil K M, Nichol K L, Simoes E A, Wright P F, Sales V M. Comparison of the safety and immunogenicity of 2 respiratory syncytial virus (rsv) vaccines—nonadjuvanted vaccine or vaccine adjuvanted with alum—given concomitantly with influenza vaccine to high-risk elderly individuals. J Infect Dis. 2008 Nov. 1; 198(9):1317-26. Falsey A R, Walsh E E. Safety and immunogenicity of a respiratory syncytial virus subunit vaccine (PFP-2) in the institutionalized elderly. Vaccine. 1997 July; 15(10):1130-2. Falsey A R, Walsh E E. Safety and immunogenicity of a respiratory syncytial virus subunit vaccine (PFP-2) in ambulatory adults over age 60. Vaccine. 1996 September; 14(13):1214-8.).

To approximate the RSV sero-status of elderly humans, boosting of RSV specific antibody and CMI responses by immunizations with RSV sF alone, RSV sF+GLA-SE or RSV sF+alum, were performed in highly RSV seropositive BALB/c mice. In addition to boosting of RSV immune responses, this study also determined if immunization with RSV sF+alum, a Th2 biasing adjuvant could alter a pre-existing Th1 immune response established by wt RSV infections as a case study on the ability of adjuvants in general to alter pre-existing Th-biased host immune response. Previous mouse studies described above were performed in RSV naïve animals using affinity purified RSV sF. In contrast, the RSV sF used in this study was purified by classical chromatography. RSV sF was given over a 1000-fold range (0.05 to 50 μg) alone or formulated with GLA-SE or alum to evaluate its ability to boost RSV immune responses in BALB/c mice previously infected twice with live RSV.

### Materials and Methods

### Study Design

One hundred three female BALB/c mice (Charles River), ages 6-8 weeks old, were divided into 13 groups. Group 1 had 7 mice and groups 2 through 13 had 8 mice. Following anaesthetization groups 1 through 12 were dosed with 1×10⁶ plaque forming units (PFU) in 100 μL of live RSV via an intranasal (IN) route on Day 0 and Day 35. Group 13 was not exposed to RSV. On Day 56, groups 1 through 11 were immunized with placebo (PBS) or vaccine article via an intramuscular (IM) route following anesthesia with isoflurane. The vaccine articles were formulated in a total of 100 μL with 50 μL given in each hind limb. Group 12 was anesthetized with isoflurane and immunized with 1×10⁶ PFU in 100 μL of live RSV via an IN route. A subset of the mice from each group were anesthesized and challenged with 1×10⁶ PFU live RSV A2 via an intranasal route on Day 84. Sera were obtained from retro orbital blood collection at study days 0, 28, 56 70 and 84, separated from whole blood and stored at −20° C. until evaluated. Spleens from 4 animals in each group were harvested for T cell assays on Day 67, 11 days post immunization, or at day 88, 4 days post challenge. Lung cytokines quantified at 4 days after challenge in individual lung homogenates by luminex assay (Milipore).

### RSV sF and Adjuvants

RSV F protein containing amino acids 1-524 of the RSV A2 F sequence was expressed from a stable CHO clone and was purified via classical chromatography methods. The RSV F protein was >90% pure and used both for animal immunizations and coating in ELISA assays. Alum (Alhydrogel, Accurate Chemical and Scientific, NJ) was used at 100 μg per vaccine dose, and adsorbed to protein by 30 minutes of mixing at room temperature. GLA in an aqueous formulation was used at 5 μg per dose. SE was used at a 2% concentration. GLA-SE was used at a dose of 5 μg GLA in 2% SE. All vaccine formulations were prepared within 2 hours of administration.

### Serum IgG, IgG1 and IgG2a ELISA

RSV-F-specific IgG antibodies were assessed using standard ELISA techniques. High binding 96 well plates were coated with purified RSV sF. After blocking, serial dilutions of serum were added to plates. The monoclonal antibody 1331H (Beeler J A, van Wyke Coelingh K. Neutralization epitopes of the F glycoprotein of respiratory syncytial virus: effect of mutation upon fusion function. J Virol. 1989; 63(7):2941-50) was used to generate a standard curve for the total IgG and IgG1 quantification and the monoclonal antibody 1308 was used to generate a standard curve for IgG2a quantification. Bound antibodies were detected using HRP-conjugated goat anti-mouse IgG, IgG1, or IgG2a (Jackson ImmunoResearch, West Grove, Pa.) and developed with 3,3′,5,5′-tetramethylbenzidine (TMB, Sigma, St. Louis, Mo.). Absorbance was measured at 450 nm on a SpectraMax plate reader and analyzed using SoftMax Pro (Molecular Devices, Sunnyvale, Calif.). Titers are reported as μg/mL of 1331H or 1308 equivalence.

### RSV Microneutralization Assay (Same as Naive Study)

RSV neutralizing antibody titers in heat-inactivated mouse sera at indicated timepoints were measured using a GFP-tagged RSV A2 micro-neutralization assay as previously described (Bernstein D I, et al. (2012) Phase 1 study of the safety and immunogenicity of a live, attenuated respiratory syncytial virus and parainfluenza virus type 3 vaccine in seronegative children. Pediatr Infect Dis J 31: 109-114). Briefly, confluent Vero cell monolayers were infected with 500 PFU of virus alone or virus pre-mixed with serially diluted serum samples, then incubated at 33° C. and 5% CO₂ for 22 hrs. Plates were washed of free virus and GFP fluorescent viral foci were enumerated using the IsoCyte image scanner (Blueshift, Sunnyvale, Calif.). Neutralizing titers were expressed as the log₂ reciprocal of the serum dilution that resulted in a 50% reduction in the number of fluorescent foci (EC₅₀ titers) as calculated using a 4-parameter curve fit algorithm.

### ELISPOT Assay (Same as Naive Studies)

Individual spleens were disrupted through a 100 micron nylon filter (Falcon) at the indicated harvest times. Viability of red blood cell depleted splenocytes was determined by ViCell and cells were resuspended at 10×10⁶ viable cells/mL in RPMI 1640 supplemented with 5% FCS, penicillin-streptomycin, 2 mM L-glutamine and 0.1% (3-mercaptoethanol (cRPMI-5) prior to use.

Mabtech (Cincinnati, Ohio) murine IFNγ ELISPOT kits were used for mouse ELISPOT assays. Pre-coated microtiter plates were blocked with cRPMI-5 prior to addition of cells and stimulants. 250,000 cells/well were incubated on blocked coated plates for 36-48 hours in triplicate with media alone, MHC II (I-E^{d})-binding peptides GWYTSVITIELSNIKE (SEQ ID NO:10) and VSVLTSKVLDLKNYI (SEQ ID NO:11) (Olson M R, Varga S M (2008) Pulmonary immunity and immunopathology: lessons from respiratory syncytial virus. Expert Rev Vaccines 7: 1239-1255)(5 μg/mL each), MHC I (H2-K^{d}) binding peptide, KYKNAVTEL (SEQ ID NO: 12) (Olson M R (2008), or ConA (5 μg/mL) as a positive control. Following incubation cells were washed away, plates were incubated with included biotinylated anti-murine IFNγ followed by SA-HRP following the kit protocol, and spots were detected with included TMB reagent. Plates were read and analyzed using a CTL ImmunoSpot reader and software (Cellular Technology Ltd).

### Cytokine Profiling (Same as Naive Studies)

Mouse cytokine/chemokine multiplex kits designed to include IFNgamma, IL-5, IL-13, IL-17 and eotaxin (Millipore, Billerica, Mass.) were used to evaluate lung homogenates. Lung homogenates were clarified by centrifugation prior to use. Assays were performed following manufacturer's instructions and plates were analyzed on a Luminex reader (Bio-Rad, Hercules, Calif.).

These experiments demonstrated that, in seropositive mice having high and low baseline seropositivity, RSV-sF boosts neutralizing antibody response, regardless of the adjuvant used or the dose of RSV-sF provided. However, formulating RSV-sF with GLA-SE elicited the strongest CD8 T cell response in seropositive mice. Additionally, formulations such as RSV sF alone or RSV sF+alum that elicted a Th2 response in naive BALB/c mice did not change the Th1 bias in seropositive animals that was elicited by the pre-exposure to RSV. In seropositive mice, administration of RSV-sF increases neutralizing antibody response, regardless of the adjuvant used or the dose of RSV-sF administered.

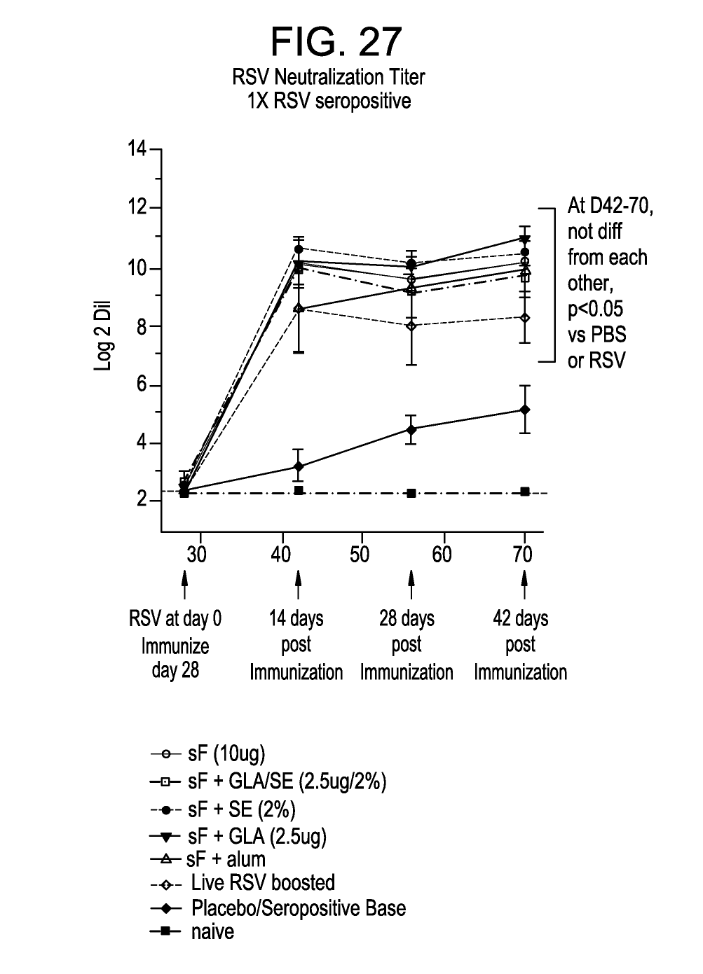

FIG. 27 is a graph showing that RSV-sF boots neutralizing antibodies in seropositive mice. The magnitude by which the titers were increased was more pronounced for animals with a lower initial neutralization titer. The titers may have been increased to a maximum neutralizing titer, which was maintained for 72 days post vaccination. Again, the increase was independent of adjuvant.

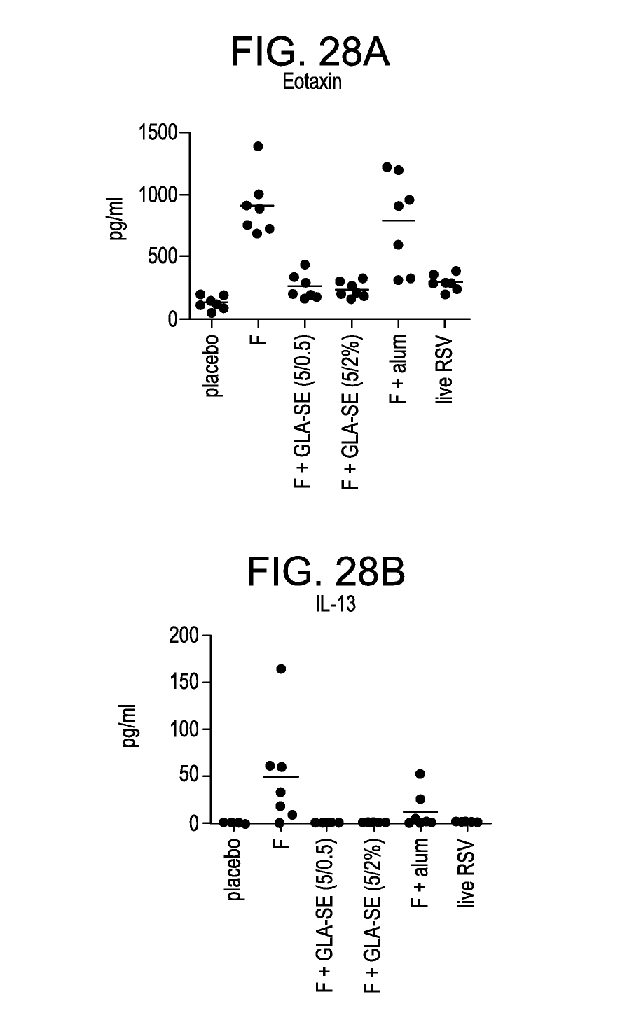

FIGS. 28 A and B are graphs demonstrating that eotaxin and IL-13 are not induced post RSVA2 challenge. Rantes is the only chemokine/cytokine that is affected by presence of adjuvant.

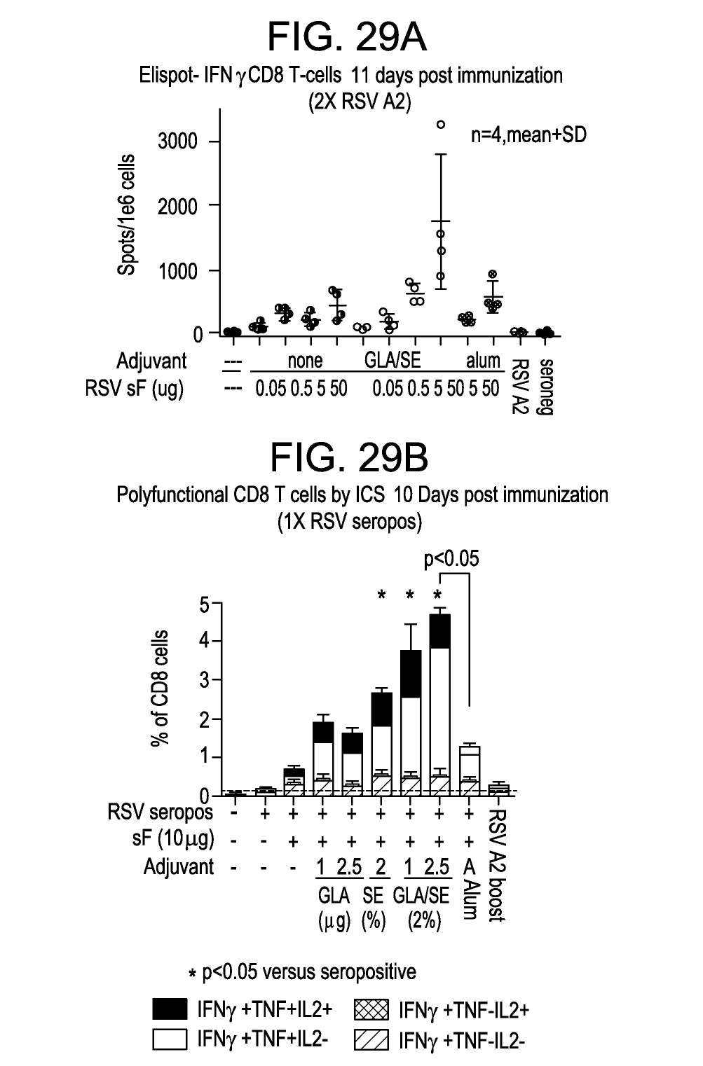

FIGS. 29A and B are graphs demonstrating that RSV-sF+GLA-SE boosts CD8 T-cell response and that the CD8 T cell response is dosage dependent. It is unknown whether the maximum response was reached with 50 μg RSV-sF. However, the formulation with RSV-sF+GLA-SE resulted in the CD8 T cell response having the greatest magnitude with a polyfunctional response.

Results

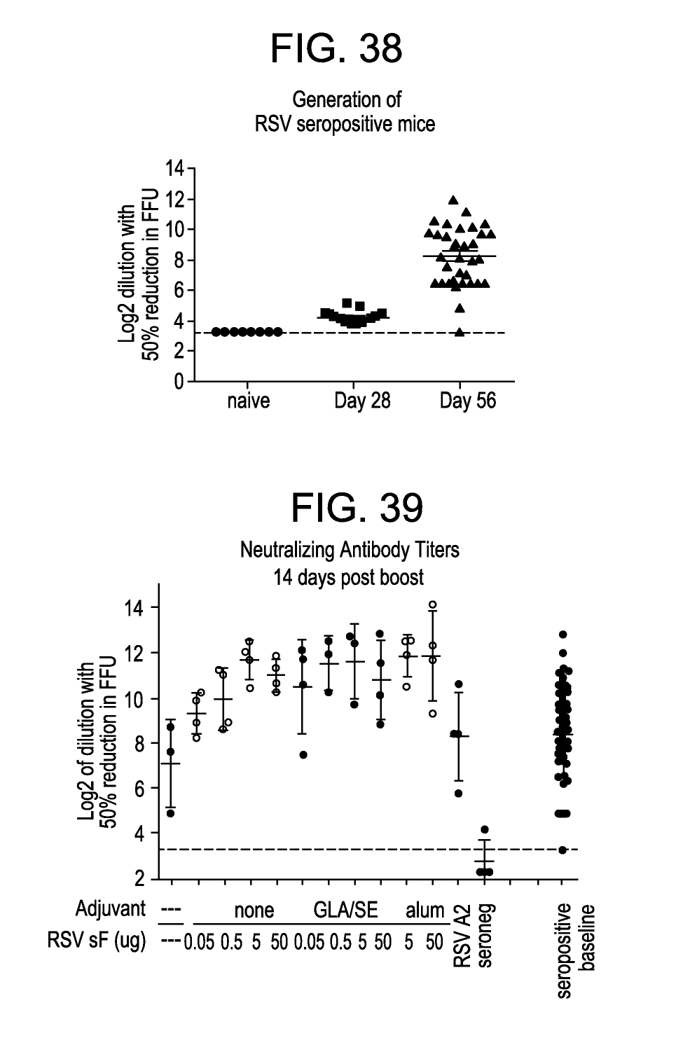

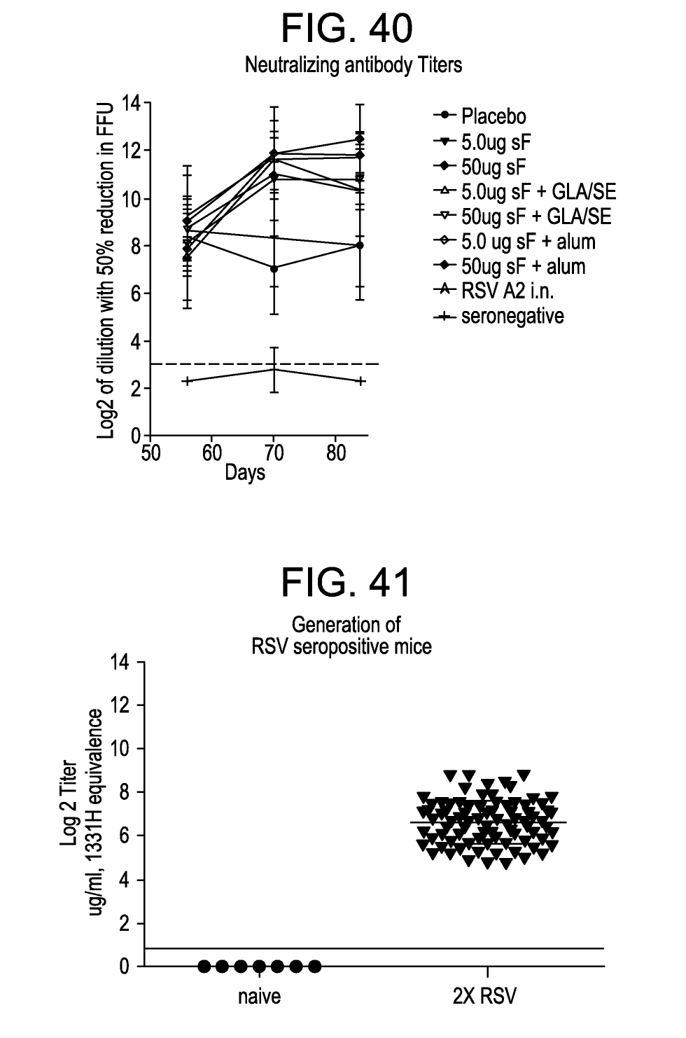

Because the respiratory tract of BALB/c mice are only semi-permissive for RSV replication, high levels of serum neutralization titers are difficult to achieve following a single intranasal dose of live RSV. To more closely approximate the level of serum neutralization titers observed in humans that have been multiply re-infected with RSV, mice were exposed to 1×106 PFU of RSV twice, on days 0 and 35. As expected, following a single dose, there were low but detectable neutralization titers in all RSV infected mice (FIG. 38). The average RSV neutralization titer was 4.2 log 2. Following the second dose, there was an approximately 16-fold boost in the average neutralization titer to 8.3 log 2. However, there with a wide range in the titers of individual mice ranging from 3.3 to 12 log 2.

To determine the ability of the various RSV sF formulations to boost the neutralization titers in these RSV seropositive mice, animals were vaccinated on day 56 and bled on days 70 and 84, representing 14 and 28 days post vaccination, respectively. FIG. 2 displays group titers at 14 days post boost. FIG. 39 shows the rise in neutralization titers over the duration of the study. The data illustrate that all RSV sF vaccine articles were able to boost neutralization titers from a group average of 8.3 log 2 to between 9.3 log 2 and 11.9 log 2 at 14 days post immunization, regardless of the presence of an adjuvant or type of adjuvant. Therefore, in RSV seropositive BALB/c mice, the RSV neutralization titers could be boosted by very small amounts of unadjuvanted RSV sF to levels that are only approximately 6-fold lower than that achieved by the highest adjuvanted RSV sF dose of 50 μg.

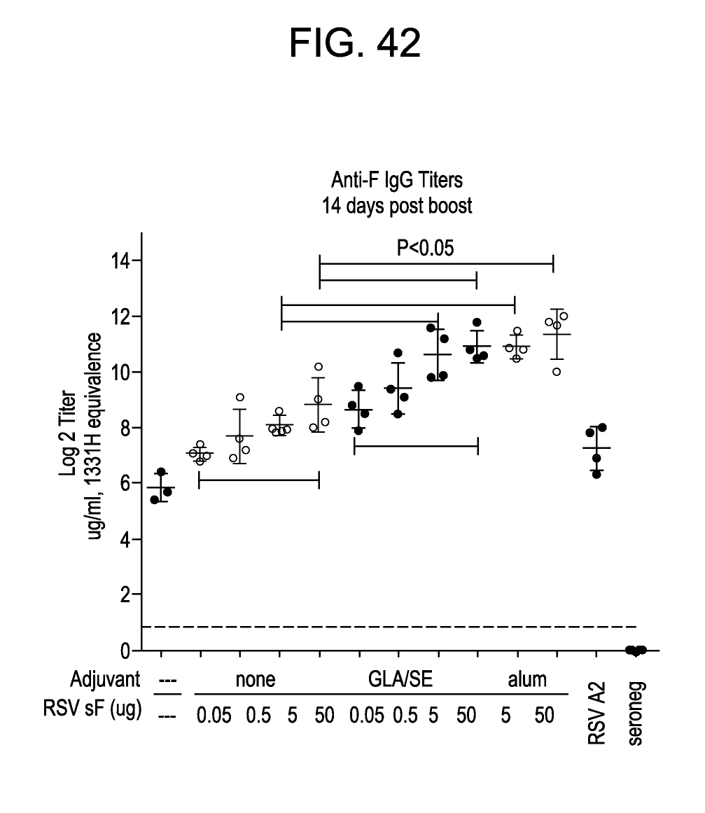

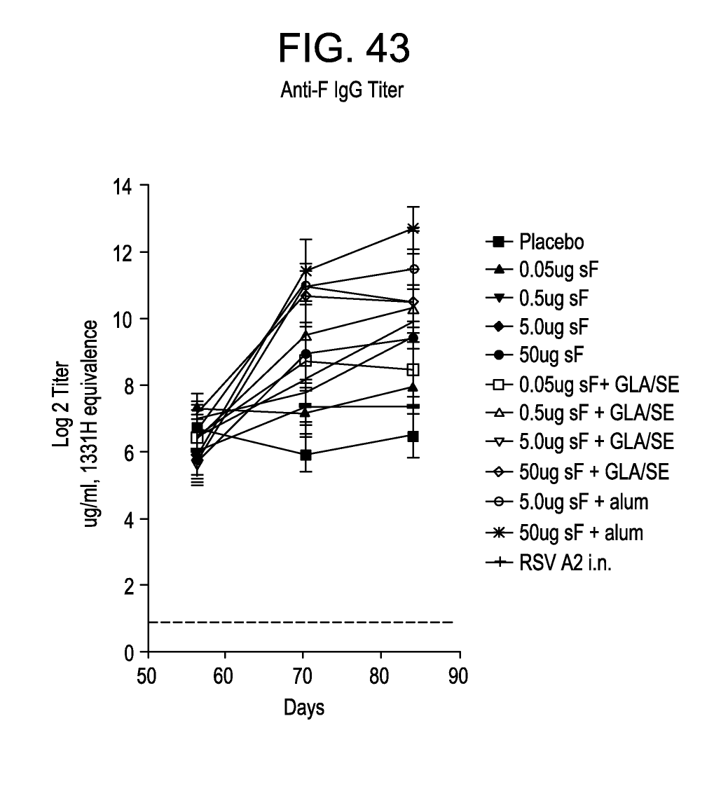

Total RSV F-specific IgG titers were measured at Day 0 prior to RSV infection and at Day 56, following two doses of RSV. FIG. 41 demonstrate that there were high levels of anti-F specific IgG titers after two serial exposures to live RSVA2 prior to immunization with the vaccine articles. FIG. 42 displays group titers at 14 days post boost. FIG. 43 shows the rise in IgG titers over the duration of the study. Unlike the neutralization titers there is a small dose response. RSV sF at 0.05 μg dose gave a boost that is statistically lower than the 50 μg RSV sF dose and RSV sF at 0.05 μg with GLA-SE gave a boost that is statistically lower than the RSV sF 50 μg dose with GLA-SE. In addition, the presence of either GLA-SE or alum also enhances the response. Both the 5 and 50 μg RSV sF groups boosted RSV F specific IgG titers to levels that are statistically lower than corresponding doses mixed with GLA-SE or alum.

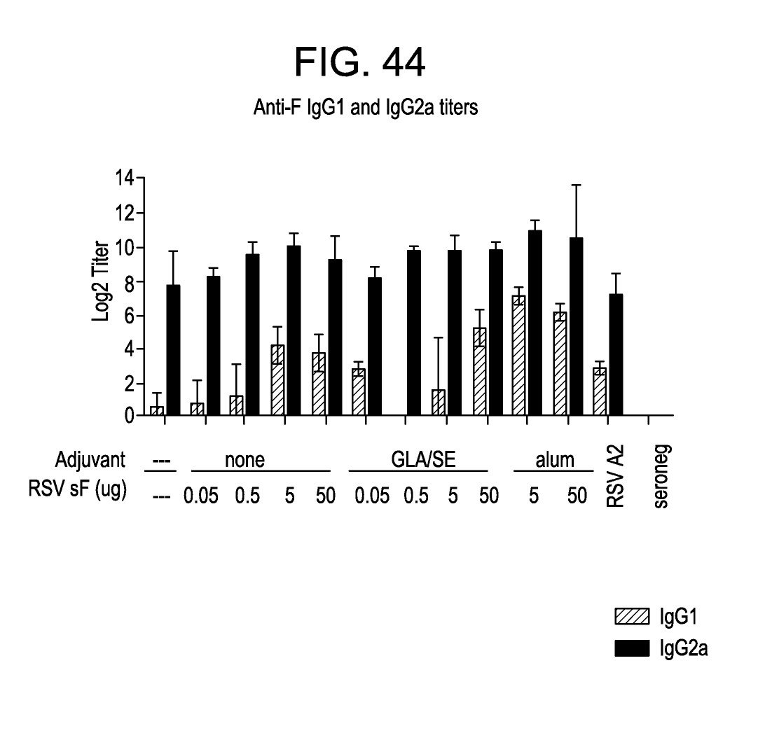

The anti RSV sF-specific IgG1 and IgG2a serum titers were measured at day 84, 24 days post-immunization (FIG. 44). In previous studies in naive mice, infection with live RSVA2 resulted in a Th1 biased response while immunization with RSV sF alone or RSV sF adsorbed on alum generated a Th2 biased response. In contrast in this study, Th-1 biased seropositive BALB/c mice maintained the Th1 bias following immunization with RSV sF alone or RSV sF adsorbed on alum. Therefore, pre-established host Th 1 skewing was not altered by immunization with RSV sF or RSV sF+alum.

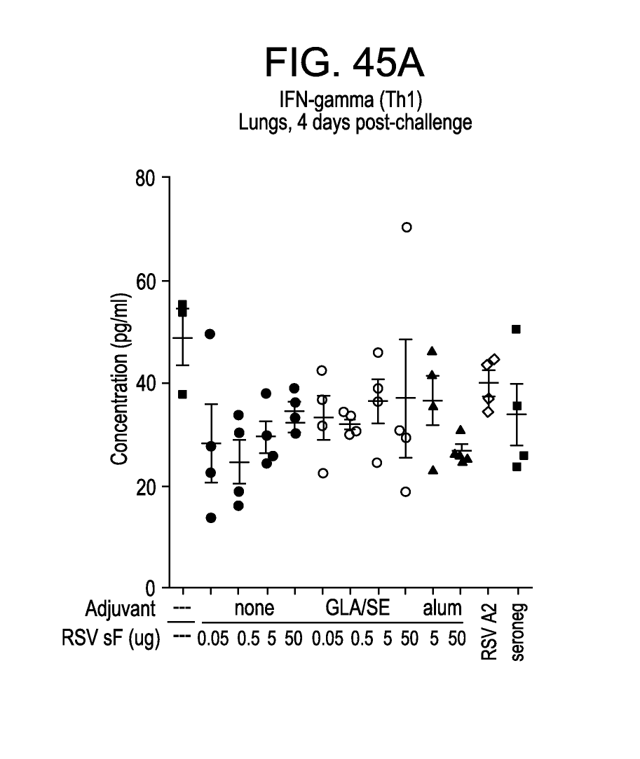

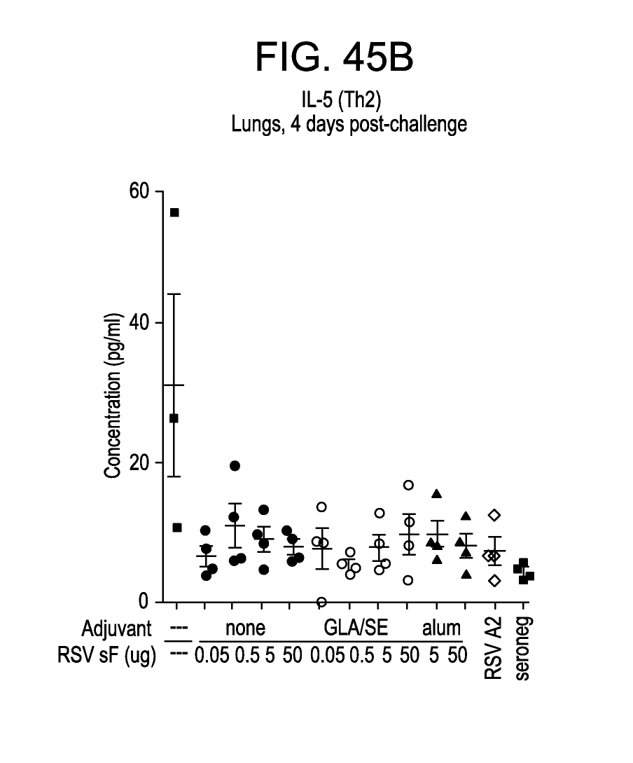

Previous immunization studies in RSV naive mice with RSV sF alone, RSV sF+GLA-SE, RSV sF+alum or primary infection with RSV resulted in high IFN γ levels at 4 days post challenge. In addition, immunization with RSV sF alone or RSV sF adsorbed on alum resulted in induction of IL-5 responses post RSV challenge, indicative of a Th2-biased response for these two groups. In this study the IFN γ and IL-5 titers in lungs were measured at day 88, 4 days post challenge with 1×10⁶ PFU RSVA2 (FIG. 45). Unlike naive BALB/c mice, immunization with RSV sF or RSV sF absorbed on alum did not set up mice to induce IL-5 in response to RSV challenge, consistent with the IgG1 to IgG2a ratio measured in the blood that indicated a Th-1 biased immune response. Therefore, RSV infected BALB/c mice appear to maintain the Th1 bias immune response established by prior RSV infection and continue to show the same Th response following immunization with RSV sF alone or RSV sF+alum.

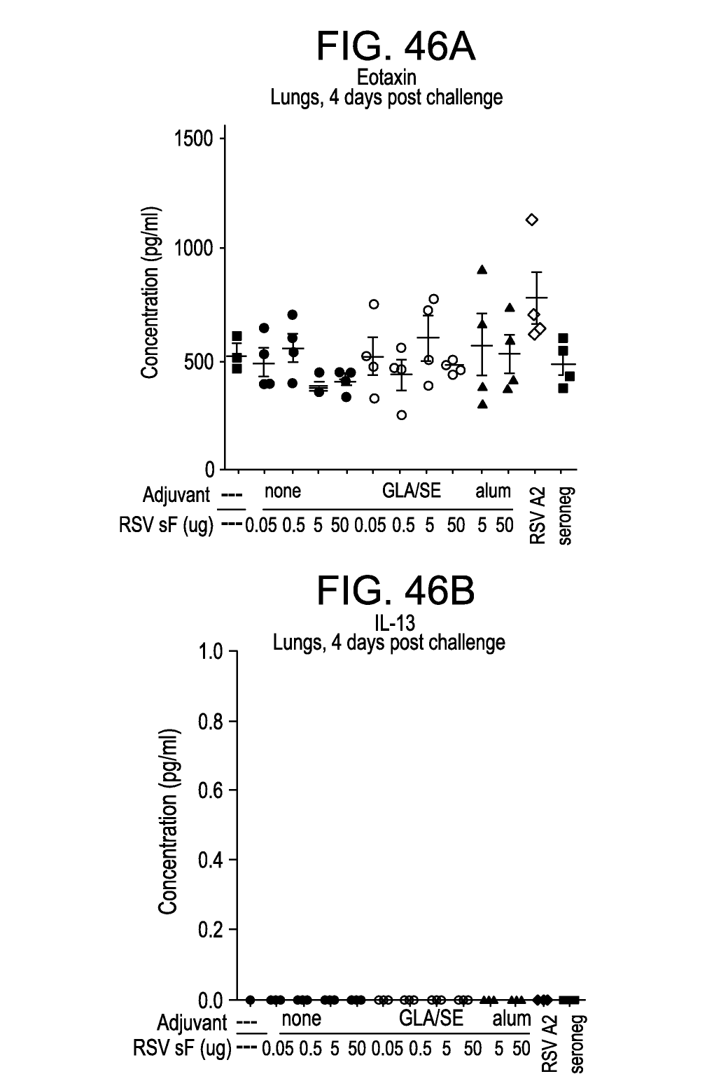

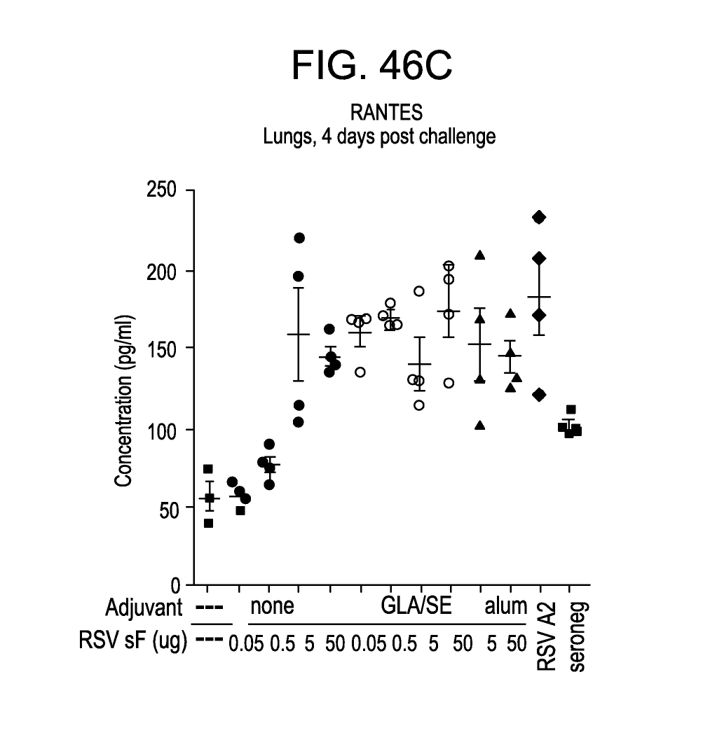

In the naive BALB/c mouse model, eotaxin and IL-13 were measured at 4 day post challenge as a surrogate immune marker for eosinophil recruitment, a potential indicator of vaccine safety. These previous studies showed that immunization with RSV sF alone or RSV sF+alum both set up mice to have eotaxin and IL-13 responses upon RSV challenge that were higher than that induced by primary RSVA2 infection. In contrast, RSV seropositive mice immunized with RSV sF alone or RSV sF+alum did not induce eotaxin or IL-13 levels higher than any of the other groups upon RSV challenge, including the cohort infected with RSV (FIG. 46).

Both the IgG1/IgG2a data and the lung cytokine data suggest that the formulation of the vaccine article does not influence the pre-existing Th-1 bias in a seropositive mouse. The only lung cytokine that was found to be differentially affected by either the RSV sF dose or the presence of an adjuvant was RANTES (FIG. 46). All adjuvanted RSV sF vaccine articles induced expression of RANTES following RSVA2 exposure but in the group immunized with RSV sF alone, the level of induced RANTES increased with increasing amounts of RSV sF.

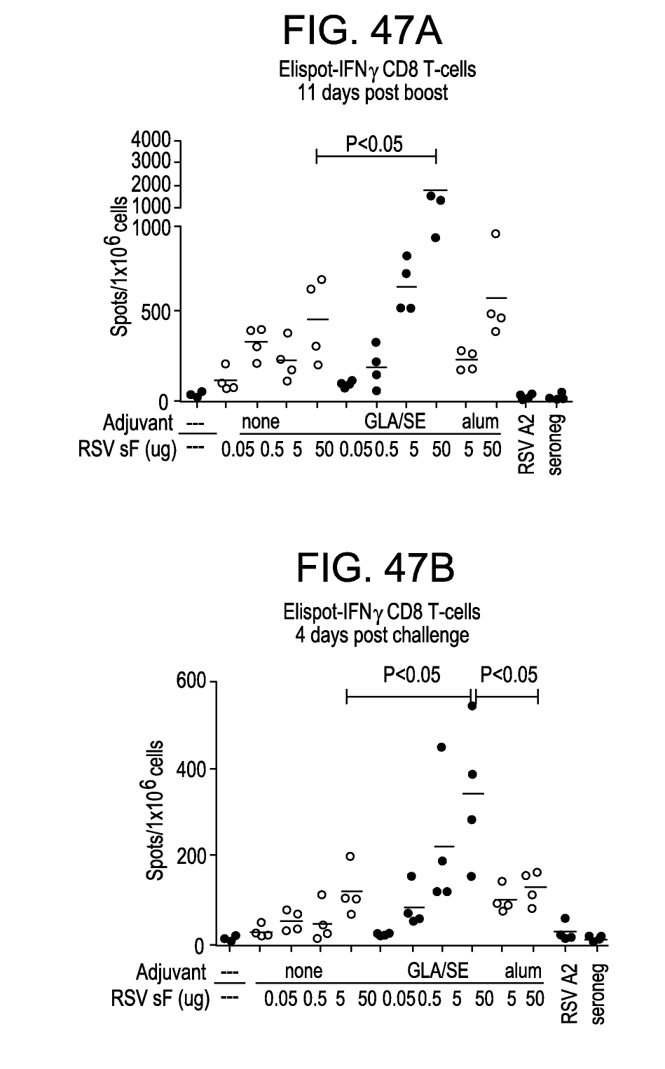

Systemic recall responses for F-specific CD8 T-cells were measured both at 11 days post immunization (Day 67) and at 4 days post challenge (Day 88) to compare magnitude of the responses elicited by the different vaccine articles. A CD8 specific, RSV F peptide was used to stimulate splenocytes for 36 hours prior to detection of IFN γ secreting cells by ELISPOT (FIG. 47). For RSV sF alone, RSVsF +GLA-SE and RSV sF+alum, increasing doses of RSV sF increased the average number of F-specific CD8 T-cells. However, unlike the serological results in which there was little difference between the responses elicited by RSV sF alone or RSV sF+GLA-SE or alum, GLA-SE was clearly differentiated as the better adjuvant for boosting CMI responses in RSV seropositive BALB/c mice.

Conclusion

Using classical chromotography purified RSV sF, this study characterized the effect of RSV sF dose (range from 0.05 to 50 μg RSV sF) on serological responses in highly RSV seropositive BALB/c mice that had been serially infected twice with live RSV. In RSV seropositive mice that showed relatively high RSV F IgG and neutralizing RSV titers, the 1000 fold range of RSV sF dose with or without adjuvant had minimal effect on boosting the neutralizing titers. The 0.05 μg dose with or without adjuvant was almost as effective as the 50 μg dose at boosting the neutralizing titers. All vaccine articles tested boosted the neutralization titers by 2 to 5 fold. For total RSV F specific IgG titers, higher doses of RSV sF with either GLA-SE or alum promoted a higher boost than 0.05 μg RSV F alone. However, this difference was modest accounting for about 5.7-fold enhancement further suggesting that boosting of serum antibodies can be achieved in the RSV seropositive mice with relatively small amount of RSV sF alone.

Since prior exposure to live RSVA2 elicits a Th1 biased response in the BALB/c mice it was of interest to determine if a known Th2 skewing vaccine article, such as unadjuvanted RSV sF or RSV sF+alum could switch the Th1 bias RSV responses to a Th2 biased response. Both the ratio of IgG1/IgG2a in the blood as well as the lung cytokine profile at 4 days post challenge suggest that immunization with RSV sF alone or RSV sF+Alum did not change the preexisting Th immune profile established by prior RSV infection. The type of immune response that RSV F+GLA/SE, a strong Th1 biasing vaccine, will generate in the Th2 biased RSV seropositive elderly population remains to be evaluated.

This study also characterized RSV sF dose as well as adjuvant on their ability to boost CD8 T-cell responses in RSV seropositive BALB/c mice. Similar to what was found in naive mice, larger doses of RSV sF did promote a higher magnitude boost than smaller RSV sF doses. In addition, RSV sF+GLA-SE resulted in the highest boost compared to the same unadjuvanted RSV sF or absorbed on alum.

### Example 2c

### RSV-F Subunit Vaccine Adjuvanted with GLA-SE in Seropositive Cotton Rats

In this study, a seropositive cotton rat model was used to evaluate how RSV sF dose affects response and whether adjuvant modulates the response following a protocol similar to that used in Example 2b.

Briefly, on Day 0, 96 cotton rats were administered 1e6pfuRSVA2 via an intrasal route. On Day 28, the animals were immunized intramuscularly with one of the following compositions: phosphate buffered saline (PBS); PBS+GLA-SE; 0.1 μg, 1.0 μg or 10 μg RSV-sF; 0.1 μg, 1.0 μg or 10 μg RSV-sF formulated GLA-SE; 10 μg RSV-sF+GLA; 10 μg RSV-sF+SE; 10 μg RSV-sF+alum; or live RSV A2. The animals were bled at D14, D28, D38, D49 and D56. The animals were then challenged at D67 with 1×106 PFU RSV A2 and spleen/lungs were harvested at D71. In another study, 64 cotton rats were administered 1×106 PFU RSV A2 via an intranasal route on Day 0. On Day 28, the animals were immunized intramuscularly with one of the following compositions: PBS; PBS+GLA-SE; 10 μg RSV-sF, 10 μg RSV-sF formulated GLA-SE; 10 μg RSV-sF+GLA; 10 μg RSV-sF+SE; 10 μg RSV-sF+alum; or live RSV A2. The animals were bled on D28 and D38.

RSV F protein containing amino acids 1-524 of the RSV A2 F sequence was expressed from a stable CHO clone and was purified via classical chromatography methods. The RSV F protein was >90% pure and used both for animal immunizations and coating in ELISA assays. Alum (Alhydrogel, Accurate Chemical and Scientific, NJ) was used at 100 g per vaccine dose, and adsorbed to protein by 30 minutes of mixing at room temperature. GLA in an aqueous formulation was used at 5 μg per dose. SE was used at a 2% concentration. GLA-SE was used at a dose of 5 μg GLA in 2% SE. All vaccine formulations were prepared within 2 hours of administration.

RSV-F-specific IgG antibodies were assessed using standard ELISA techniques. High binding 96 well plates were coated with purified RSV sF. After blocking, serial dilutions of serum were added to plates. Bound antibodies were detected using HRP conjugated chicken anti cotton rat IgG antibody (Immunology Consultants Lab) and developed with 3,3′,5,5′-tetramethylbenzidine (TMB, Sigma, St. Louis, Mo.). Absorbance was measured at 450 nm on a SpectraMax plate reader and analyzed using SoftMax Pro (Molecular Devices, Sunnyvale, Calif.). Titers are reported as the absorbance at a 1:1000 serum dilution or the log 2 endpoint titer using a cutoff of 2 times the mean of the blank wells. Site specific antibodies were quantified via a competition ELISA assay. Briefly, high binding 96 well plates were coated with purified RSV sF. After blocking, serial dilutions of serum were mixed with a constant concentration of biotinylated antibody that recognized Site A, Site B or Site C (Beeler J A, van Wyke Coelingh K. Neutralization epitopes of the F glycoprotein of respiratory syncytial virus: effect of mutation upon fusion function. J Virol. 1989; 63(7):2941-50). The percent competition for individual sera at a representative dilution was calculated (100×[1-{seraOD/mAbODmean}]). The microneutralization titers were determined as described previously for the naive mouse studies.

Results

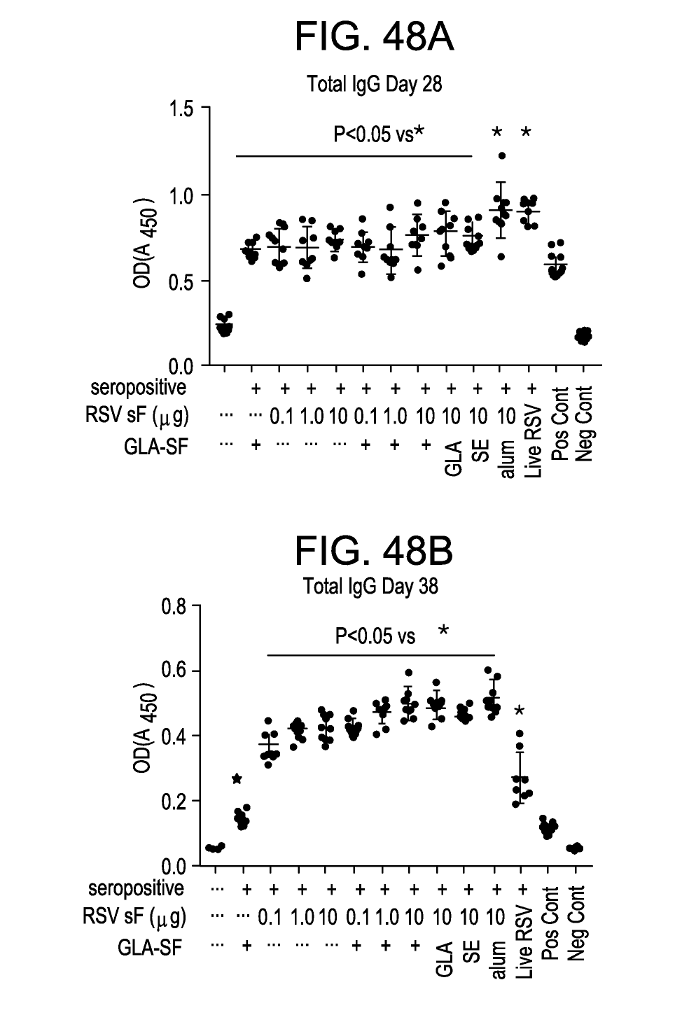

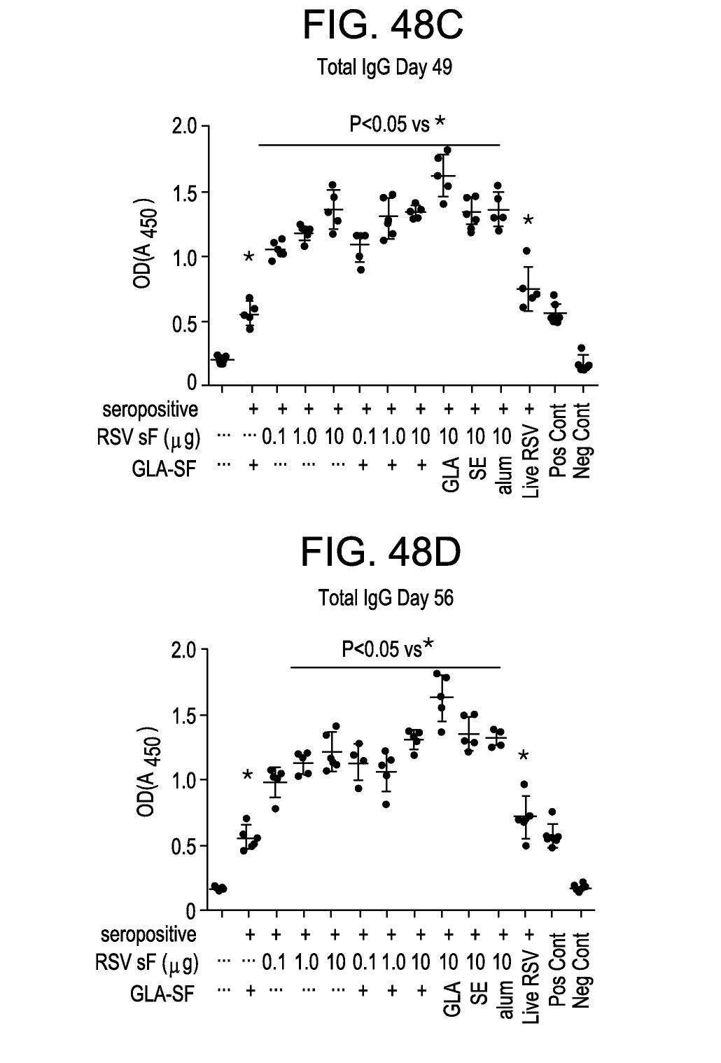

The level of total RSV F-specific IgG titers were measured 28 days following RSV infection to establish the baseline antibody titers prior to immunization and at Days 38, 49 and 56 to measure the boost in antibody titers post-immunization. On Day 28 there were significant levels of RSV F specific IgG after one exposure to live RSVA2. The data for Days 38, 49 and 56 demonstrate that all groups vaccinated with RSV sF, irrespective of dose boosted RSV sF specific IgG titers and boosting was not significantly enhanced by the presence of adjuvant (FIG. 48). On Days 38 and 49, the average A450 OD values at the 1:1000 dilutions were all significantly higher for these groups compared to placebo immunized group and the group that received a second exposure to live RSVA2. On Day 56, only the 0.1 μg RSV sF dose with no adjuvant was not statistically different from both the seropositive/placebo group and the group that received a second live dose of RSVA2. The trends for Day 38 and Day 49 suggest that 100-fold more RSV sF (10 μg vs 0.1 μg) results in minimally higher titers at the highest RSV sF dose for RSV sF ±GLA-SE. Overall these data suggest that neither the dose of RSV sF nor the presence of an adjuvant greatly affects the boost in RSV F specific serum titers, supporting similar conclusions in seropositive BALB/c mice.

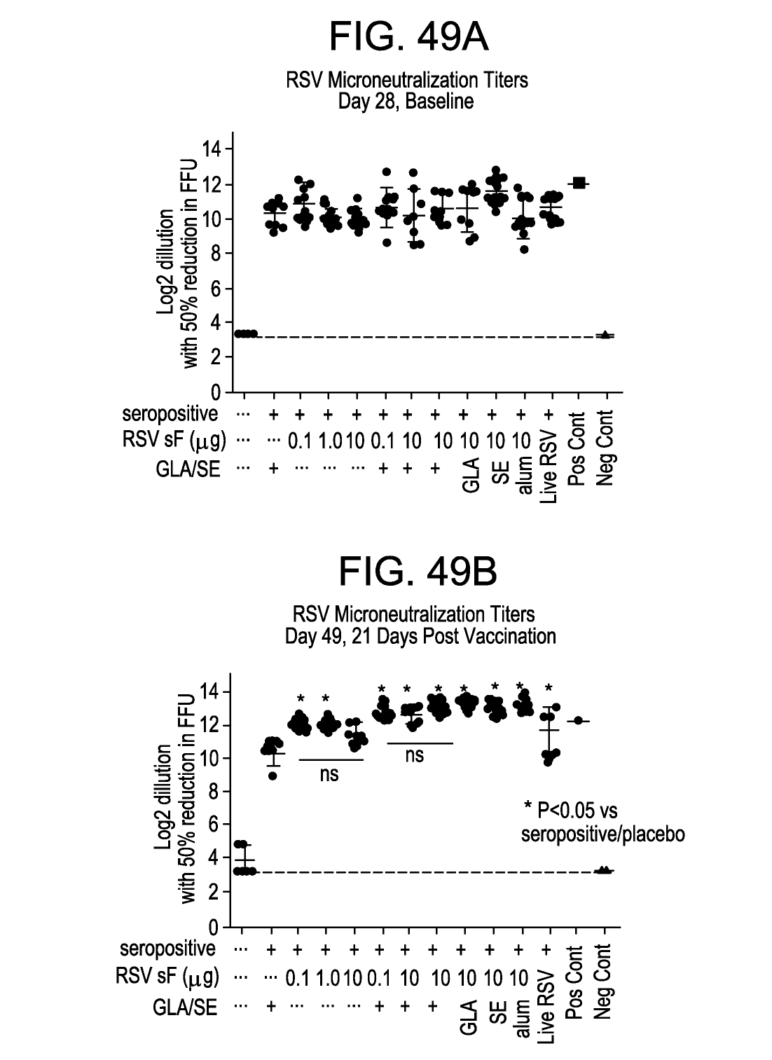

The level of RSV neutralizing antibody titers was measured on Day 28 to establish baseline neutralization titers and at Days 38, 49 and 56 to measure the boost in neutralizing antibody titers post-immunization. On Day 28 mean averages for each seropositive group were at least 10 log 2 (FIG. 49). Since cotton rats are more permissive for RSV replication, the neutralization titers following a single infection with RSV results in considerably higher mean titers than that observed in BALB/c mice. In BALB/c mice the average neutralizing titers following a single infection with 1×10⁶ PFU of live RSVA2 range between 4 log 2 and 6 log 2.

The neutralizing titers for Day 49, 21 days post-immunization, indicate that titers were boosted to mean averages between 11.4 and 13.1 (FIG. 49). All groups except the RSV sF (10 μg) cohort were boosted to titers significantly higher than the seropositive/placebo cohort. There was no statistical difference between any of the no adjuvant groups or between any of the RSV sF+GLA-SE groups, suggesting that increasing the dose of RSV sF from 0.1 to 10 g had no effect on the mean average neutralizing titer following immunization.

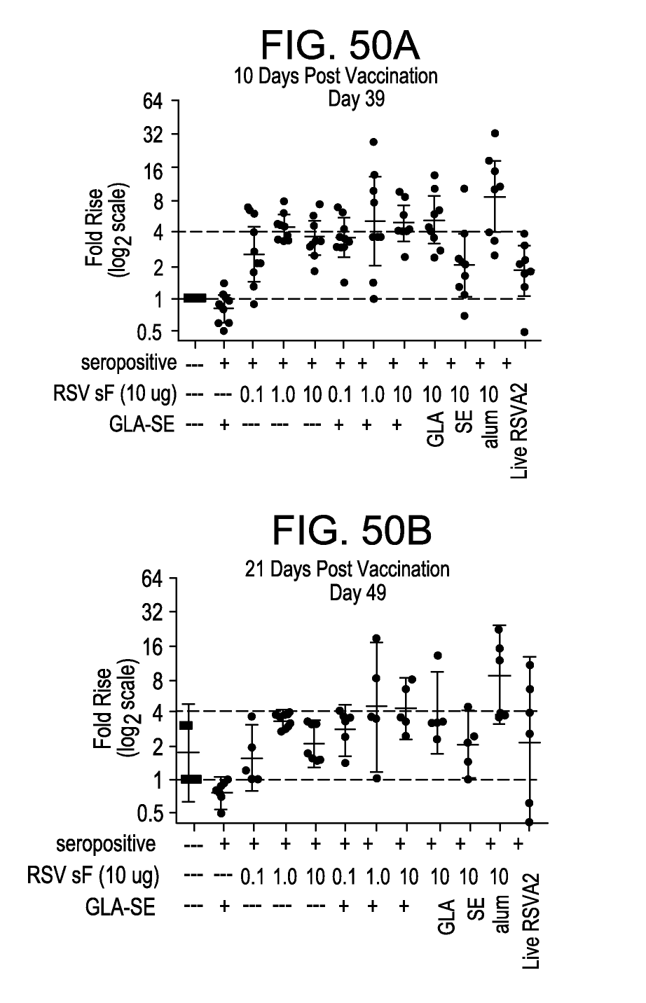

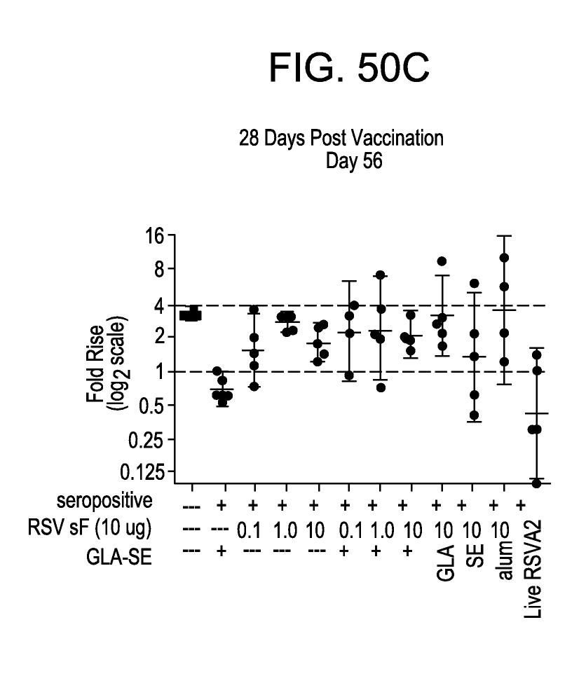

To evaluate the magnitude of the boost in neutralization titers, the fold rise in baseline titer for each animal at 10, 21 and 28 days post immunization were calculated (FIG. 50). All groups immunized with RSV sF with or without adjuvant had geometric mean average rises higher than the seropositive/placebo vaccinated group, however due to the wide spread in the data only the groups immunized with 1 μg RSV sF+GLA-SE and 10 μg RSV sF+alum were significantly higher than the seropositive/placebo vaccinated group at 10 days post immunization. At 21 days post immunization only the 10 μg RSV sF+alum group was significantly higher than placebo. Only RSV sF (10 μg), RSV sF (1 μg)+GLA-SE, RSV sF (10 μg)+GLA-SE, RSV sF (10 μg)+GLA and RSV sF (10 μg)+alum at 10 days post immunization had geometric mean rises of 4-fold or greater. This small to moderate boost in the neutralizing titers is likely due to the high baseline titers of 10 log 2. This titer is close to the maximum achievable RSV titer in cotton rat. Over all these data suggest the dose of RSV sF with or without adjuvant have minimal effects on boosting neutralizing titers, supporting the total RSV sF specific IgG results. The minimal boost is likely due to the fact that baseline titers were close to the maximum achievable RSV titers in cotton rats. Similar conclusions were made in the BALB/c seropositive animal model.

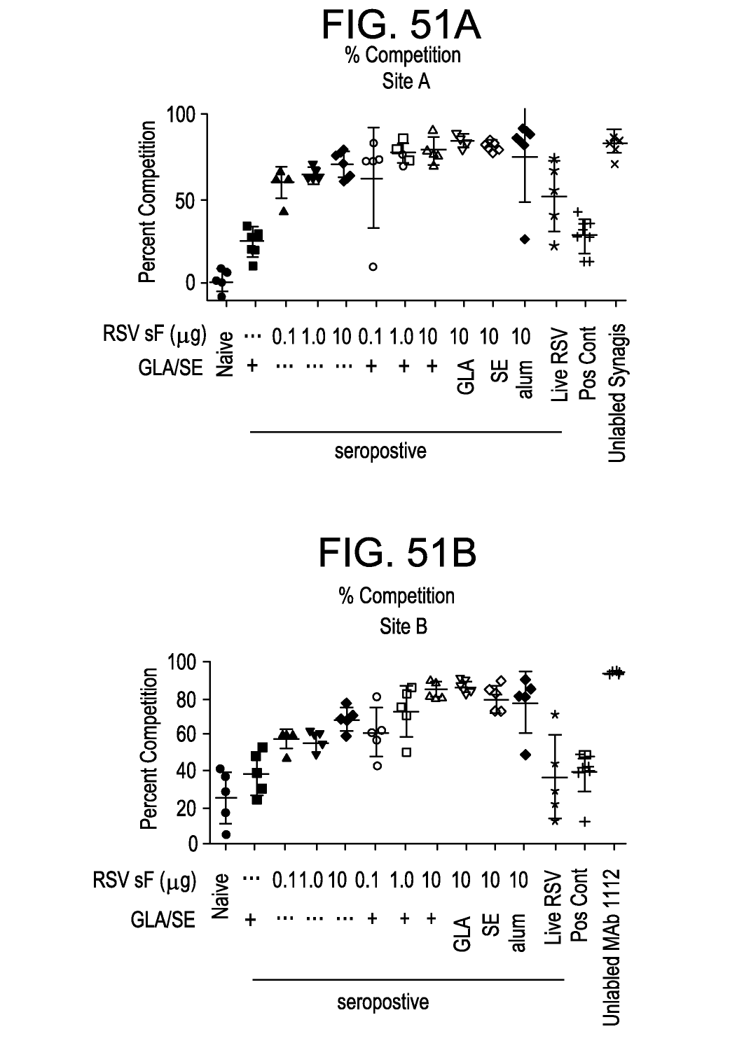

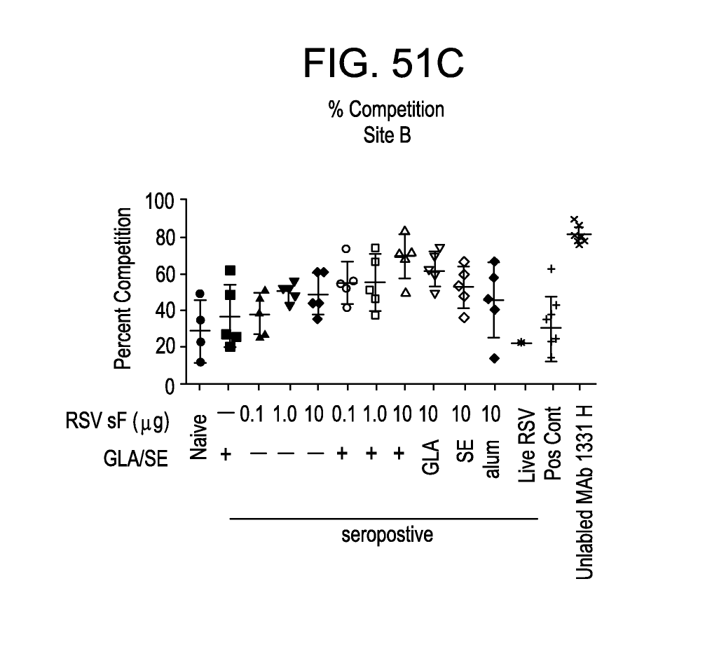

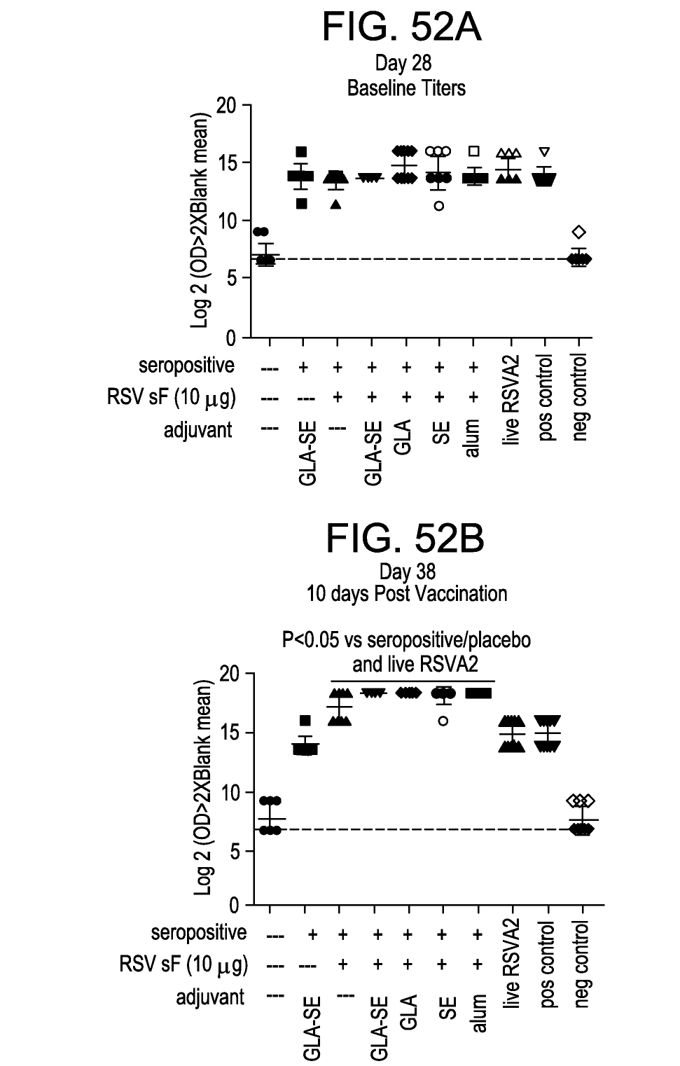

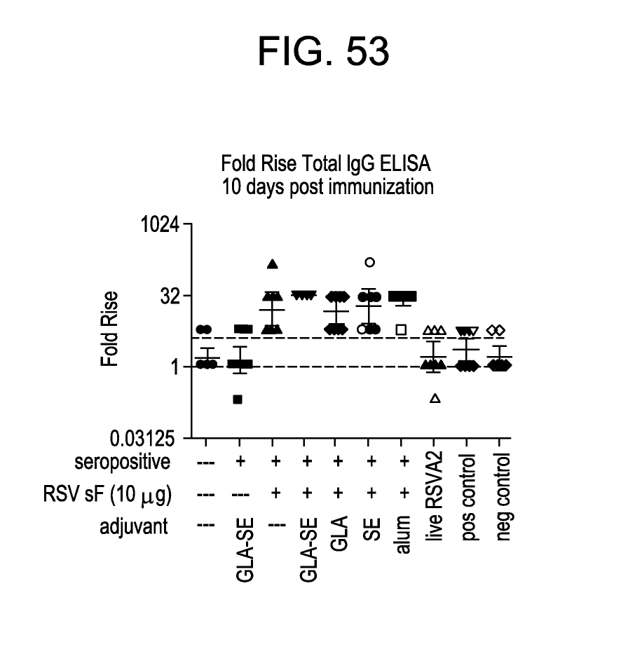

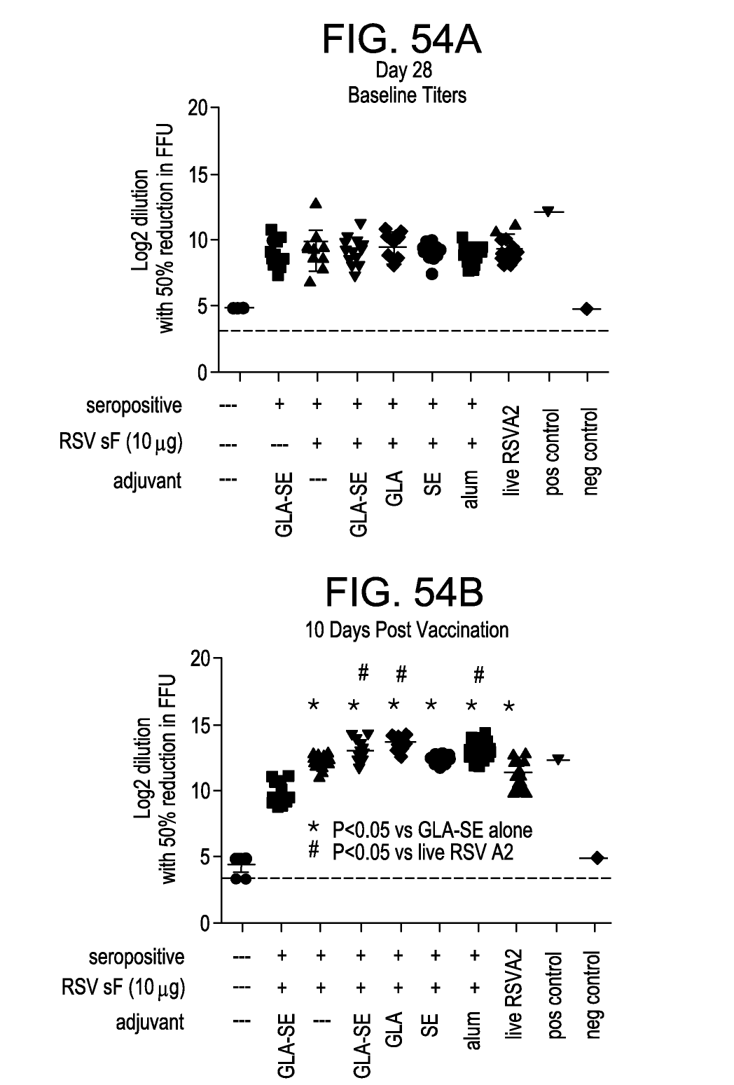

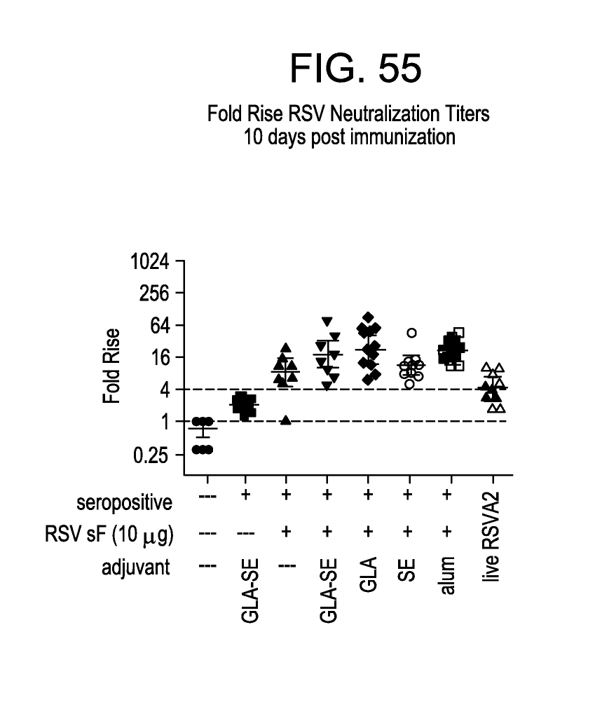

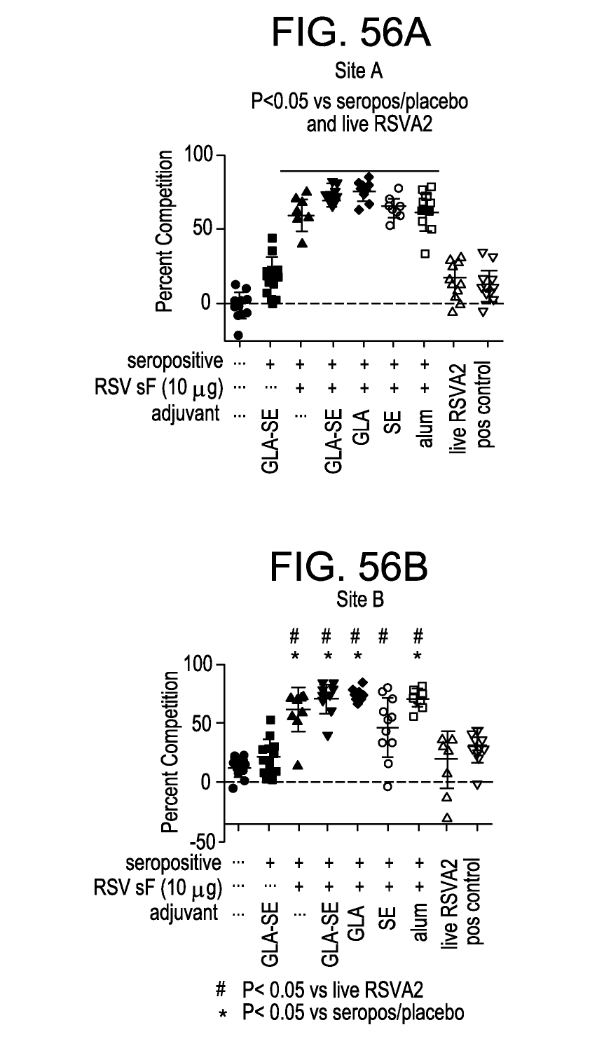

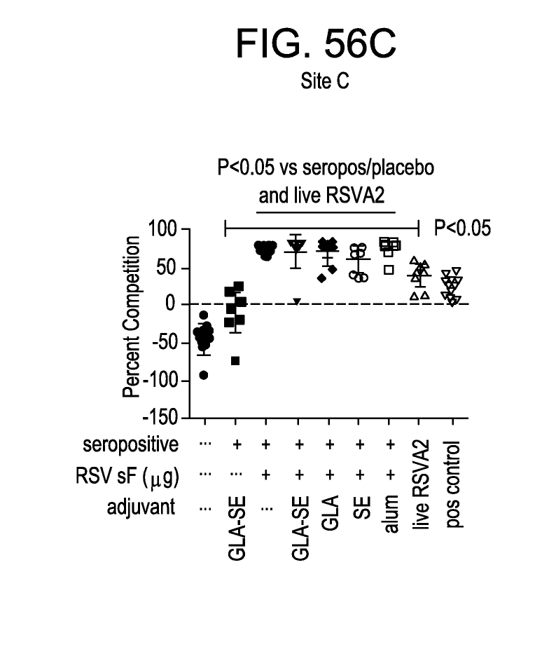

Neutralizing monoclonal antibodies (Mabs) specific for the RSV F protein have been generated and mapped to 3 major sites, Site A, Site B and Site C (Beeler J A, van Wyke Coelingh K. Neutralization epitopes of the F glycoprotein of respiratory syncytial virus: effect of mutation upon fusion function. J Virol. 1989; 63(7):2941-50). One Site A Mab (Synagis®) one site B (1112) and one Site C Mab (1331H) were each utilized in a competition ELISA to measure the relative amounts of antibodies generated to Site A, Site B or Site C in the cotton rats following the immunizations (FIG. 51). The mean averages suggest that both RSV sF alone and RSV sF with any of the adjuvants boost antibody responses to specific neutralizing sites better than placebo or a second exposure to RSV A2. In this assay the differences between 0.1, 1.0 and 10 g RSV sF cohorts with or without GLA-SE were not significantly different however the trends suggest that higher doses of RSV sF and the presence of an adjuvant may be beneficial for boosting site specific antibody responses.

In the second seropositive cotton rat study the level of total RSV F-specific IgG titers were measured 28 days following RSV infection to establish the baseline antibody titer prior to immunization and at Day 38 to measure the boost in antibody titers post-immunization. On Day 28 there were significant levels of RSV F specific IgG after one exposure to live RSVA2 (FIG. 48). All groups reach mean titers between 13.4 and 14.7 log 2. The data for Days 38 demonstrate that all groups vaccinated with RSV sF, irrespective of the adjuvant, boosted RSV sF specific IgG to titers significantly higher than placebo (PBS+GLA-SE) or a second dose of RSVA2.

To evaluate the magnitude of the boost in serum IgG titers, the fold rise from baseline titer for each animal at 10 days post immunization were calculated (FIG. 49). All groups immunized with RSV sF with or without adjuvant had geometric mean rises greater than 4-fold and ranged between 12.0 and 25.0. The control groups such as the naive and seropositive/placebo group as well as the group that received a second dose of live RSV A2 did not have a boost in serum titers and had calculated fold rises less than 2.

The level of RSV neutralizing antibody titers was measured on Day 28 to establish baseline neutralization titers and at Day 38 to measure the boost in neutralizing antibody titers post-immunization. On Day 28 averages for each seropositive group were at least 9 log 2 and ranged between 9.0 log 2 and 9.5 log 2 (FIG. 50). In the previous seropositive cotton rat study the average neutralizing titers on Day 28 were between 10.1 and 11.7. Since cotton rats are more permissive for RSV replication, the neutralization titers following a single infection with RSV results in considerably higher mean titers than that observed in BALB/c mice. In BALB/c mice the average neutralizing titers following a single infection with 1×10⁶ PFU of live RSVA2 typically range between 4 log 2 and 6 log 2.

The neutralizing titers for Day 38, 10 days post-immunization, indicate that titers were boosted to averages between 12.3 and 13.9 (FIG. 50). Similar post-immunization titers were observed in the previous seropositive cotton rat study. All RSV sF groups were boosted to significantly higher titers than the placebo group. Unlike the data for the serum IgG titers, the live RSV A2 group also had a boost in neutralizing titers that were significantly higher than the placebo group. In addition, the RSV sF+GLA-SE, RSV sF+GLA, and RSV sF+alum groups were boosted to titers higher than RSV A2. To evaluate the magnitude of the boost in neutralization titers, the fold rise from baseline titers for each animal at 10 days post immunization were calculated (FIG. 51). All groups immunized with RSV sF with or without adjuvant had mean fold rises between 8.1 and 21.9, a range similar to the fold-rise seen with the serum IgG titers. Unlike in the previous seropositive cotton rat study, the rise in neutralization titers was easier to observe since the starting baseline titers were lower at 9 log 2 compared to 10 log 2 and the variability in each group was smaller in this study. Over all these data suggest that the presence of an adjuvant has minimal effects on boosting neutralizing titers since mean RSV sF+adjuvant neutralization titers were only 2-3 fold higher than RSV sF alone. These data also support the total RSV sF specific IgG data. Similar conclusions were also made in the seropositive BALB/c mice studies.

Neutralizing monoclonal antibodies (Mabs) specific for the RSV F protein have been generated and mapped to 3 major sites, Site A, Site B and Site C (Beeler J A, van Wyke Coelingh K. Neutralization epitopes of the F glycoprotein of respiratory syncytial virus: effect of mutation upon fusion function. J Virol. 1989; 63(7):2941-50). One Site A Mab (Synagis®) one site B (1112) and one Site C Mab (1331H) were each utilized in a competition ELISA to measure the relative amounts of antibodies generated to Site A, Site B or Site C in the cotton rats following the immunizations (FIG. 51). Both the RSV sF alone and RSV sF with any of the adjuvants significantly boosted antibody responses to these specific neutralizing sites better than placebo or a second exposure to RSV A2. The only exception was with RSV sF+SE, in which the boost in Site B titers was significantly higher than a second dose of RSV A2, but was not statistically higher than the placebo group. Interestingly, the only site in which the second dose of RSV A2 boosted titers significantly higher than the placebo was for Site C.

Conclusion

Using classically purified RSV sF, this study characterized the effect of RSV sF dose over a 100-fold range (0.1 to 10 μg RSV sF) as well as the effect of adjuvant on serological responses in RSV seropositive cotton rats. Unlike the naive animal models, the RSV sF dose had minimal to no effect on the magnitude of the boost in total IgG, site specific responses or total neutralizing titers when dosed either with or without the adjuvant. Likewise the presence of any of the adjuvants at the highest RSV sF dose also had little to no effect on adjuvanting the magnitude of the responses further.

### Example 3

### RSV-sF Immunogenicity in Naïve Sprague Dawley Rats

This study evaluated the immunogenicity of a RSV-sF vaccine formulation in Sprague Dawley rats, a model routinely used for toxicology studies in drug and vaccine development. The goals of this study were: (A) to confirm that unvaccinated Sprague Dawley rats support RSV A2 replication in the lung and nose, and identify the day of peak RSV replication; (B) to quantify the level of F-specific humoral, cellular, and protective immune responses in naive Sprague Dawley rats when dosed with either 10 μg or 100 μg RSV sF with GLA-SE at 2.5 μg/2% SE; (C) to determine whether the dose of RSV sF affects the level of RSV-SF-induced humoral, cellular, and protective immune responses in naive Sprague Dawley rats; and (D) to demonstrate whether GLA-SE activity is required to induce humoral, cellular, and protective immune responses to RSV sF in naïve Sprague Dawley rats.

Viral replication of RSV A2 virus in the nose and lungs following intranasal inoculation was demonstrated in this animal model. RSV sF protein was produced from stably transfected Chinese hamster ovary (CHO) cells and column purified. 10 or 100 μg RSV sF unadjuvanted or adjuvanted with a 2.5 μg/2% dose of GLA-SE were administered to female Sprague Dawley rats intramuscularly at Day 0 and Day 22, Serological anti-F antibody responses and RSV neutralizing antibody responses were measured at Day 14, 22, and 42 following vaccination in all animals (n=4-6/group). F-specific T-cell responses were measured at Day 46, 4 days post RSV challenge in all animals (n=3-4/group). Local protective immunity post RSV challenge was demonstrated by the clearance of RSV-From the lung and the nose 4 days post challenge. This study showed that RSV-F-specific humoral immune responses were induced by both doses of antigen with and without adjuvant, while RSV-F-specific cellular immune responses were antigen- and adjuvant-dependent. The humoral and cellular immune responses induced by an RSV sF+GLA-SE vaccine candidate in Sprague Dawley rats provide full protection from RSV challenge in both the lung and the nose.

The vaccine composition contained purified RSV soluble F (sF) protein adjuvanted with Glucopyranosyl Lipid A/Stable Emulsion (GLA-SE) (Immune Design Corporation, Seattle, Wash.) for administration by intramuscular injection. Recombinant RSV sF protein was generated from a stable clonal Chinese hamster ovary (CHO) cell line. Classical column purification methods were used to purify RSV sF for this study.

An ideal toxicology animal species is one that (i) responds to the vaccine antigen and adjuvant with all the key immunological responses, (ii) is susceptible to the vaccine targeted pathogen, and (iii) will accommodate delivery of the full human dose. The toxicology model should demonstrate F-specific humoral immune responses, F-specific T cell responses, and be permissive for RSV infection in the unvaccinated state but protected from RSV challenge once vaccinated. Sprague Dawley rats are a standard toxicology species that can be dosed with up to 500 μL intramuscularly. In this study, we confirmed the replication of the RSV A2 strain in naive Sprague Dawley rats and found that RSV-sF induced humoral and cellular immunity that protects against RSV challenge, therefore satisfy all the criteria for a suitable toxicology model for evaluating RSV vaccine candidates.

An initial study was conducted to confirm RSV A2 replication and to determine the day of peak virus titer following RSV A2 infection in naive rats. 5 cohorts of RSV naïve female SD rats were infected intranasally with 2×10⁶ pfu RSV A2. On Days 1, 4, 6, 8, and 14 following infection, lungs and noses were harvested separately from 5 euthanized rats per group, homogenized on the same day and titered for RSV by plaque assay. This study showed that the day of peak virus replication was 4 days after RSV infection. No additional assays were performed in this study.

In a subsequent study, the immunogenicity and protection following a prime-boost regimen of RSV-SF was evaluated. 40 naive female Sprague Dawley rats were divided into designated vaccine cohorts of 5-6 animals per cohort. Briefly, test groups were given RSV sF (10 μg or 100 μg per animal) without adjuvant or RSV sF (10 μg or 100 μg per animal) with GLA-SE (2.5 μg in 2% SE). Negative control groups were dosed with placebo (PBS buffer) or adjuvant GLA-SE (2.5 μg/2%) without RSV sF. The positive control group was inoculated intranasally with 2×10⁶ pfu live RSV A2. Groups 1-6 were inoculated IM with 500 μL of designated vaccine article on Day 0 and Day 22, while Group 7 was inoculated IN with 200 μL of RSV A2 virus on day 0 only. All animals were challenged IN on day 42 with 2×10⁶ pfu live RSV A2 virus. Rats were euthanized at 4 days post challenge on Day 46, the day of peak viral replication determined from Study 1. Lungs (excluding 1 lobe which was formalin-fixed) and noses were homogenized and quantified for viral titers.

Reactogenicity of the adjuvanted vaccine formulations was assessed by direct observation of the rats following inoculation and by tracking animal weights 3 times per week over the course of the study (Data not shown).

Serological responses to vaccination were evaluated at 6 hours post immunization, D22, and D42 for all animals and at Day 14 for a subset of 3 animals per group. Animals were lightly anesthetized with isoflurane and bled intraorbitally. Serum was separated and stored at −20° C. and thawed for testing. Serum obtained 6 hours post-immunization was evaluated for cytokine titers by multiplexed ELISA. Serum from Days 14, 22, and 42 were measured for total anti-F IgG ELISA endpoint dilution titers. Day 42 serum was evaluated for the specific contribution of IgG1, IgG2a, and IgG2b anti-F responses by ELISA endpoint dilution titers. Serum RSV neutralization titers were determined on Days 22 and 42 by a RSV A2-GFP microneutralization assay.

Systemic cellular immune responses to vaccination were evaluated in all available animals at Day 46, 4 days post RSV challenge. For each of the groups, individual splenocyte samples were prepared. T-cell readouts were assessed by ELISPOT counts of IFNγ-secreting cells following a 36-48 hour restimulation with RSV sF. Significance was calculated using GraphPad Prism 1-way ANOVA with either Tukey or Bonferroni post test with a significance cutoff of p<0.05.

Test articles for IM administration were formulated to achieve the desired final amount of antigen and adjuvant in a 500 μL dose. The order of addition was as follows: PBS was added first, then GLA-SE adjuvant (when used) at a 1:3 final dilution, then RSV sF antigen (when used) at either a 1:500 final dilution (for a 10 μg dose) or a 1:50 final dilution (for a 100 μg dose). Formulated test articles were mixed by vortexing for 30 seconds and stored at 4° C. for up to 15 hours before administrating to animals. Stored test articles were thoroughly mixed by vortexing prior to transfer to ACF staff for administration to animals.

Live RSV A2 for IN inoculation and challenge was prepared less than 1 hour prior to administration to animals. RSV A2 aliquots were thawed on ice. For a 2×10⁶ pfu dose in 200 μL, 120.4 μL viral stock at 1.66×10⁷ pfu/mL was diluted with 79.6 μL Optimem plus 1×SP. An overage of 300 μL was prepared and transferred to ACF staff on wet ice for animal inoculations.

Residual vaccine formulations were subjected to Western blot analysis with an anti-F mAb (palivizumab) to confirm lack of RSV sF in the negative controls and presence of equivalent amounts of RSV sF in Groups 3 and 5 and in Groups 4 and 6 (data not shown). All test articles not consumed by western blot analysis were discarded.

Discussion

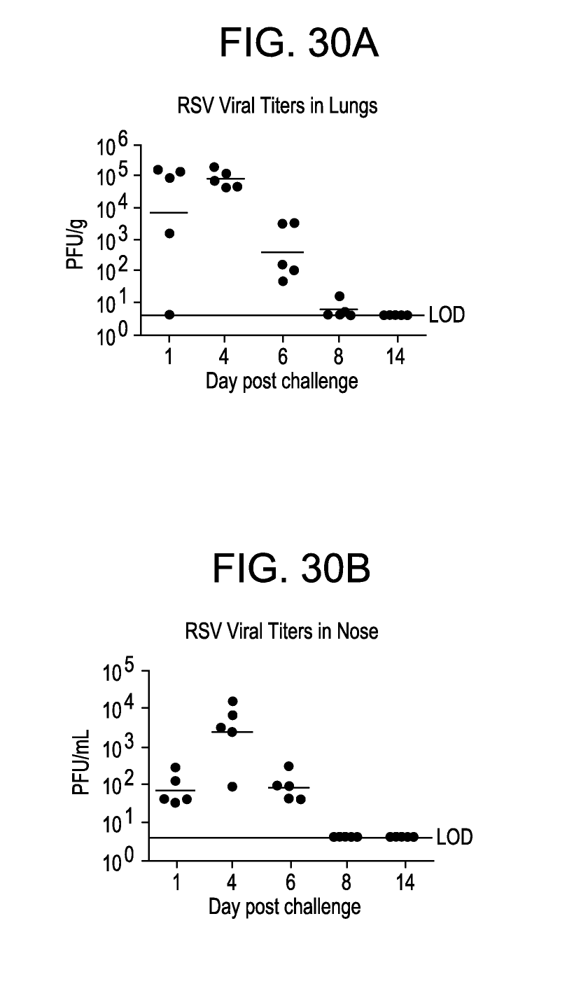

In the initial study to investigate the time course of RSV A2 strain replication in the lung and nose of Sprague Dawley rats, 25 rats were challenged IN with 2×10⁶ pfu of RSV A2 virus on Day 0. RSV viral titers were measured in homogenized lungs and noses harvested on Days 1, 4, 6, 8, and 14 post challenge. RSV viral replication was detected on Days 1, 4, and 6 in all tested animals and peaked at Day 4 in both the lung and the nose (FIG. 30). At Day 4 post challenge, peak viral loads averaging ˜10⁵ pfu/g of lung and ˜10^{3.4} pfu/mL of nose homogenate were detected. By Day 6, virus titer had decreased by about half. Only 1 of 5 animals had any detectable viral titers in the lung on day 8, and this animal had no detectable titers in the nose. Therefore, for the RSV-SF vaccine challenge study in Sprague Dawley rats, lungs and noses were harvested at Day 4 post RSV challenge which represented the day of peak virus replication.

Vaccines were prepared and given at Day 0 to all animals. Groups 1-6 received booster vaccines at Day 22. All vaccines were well tolerated with no reports of injection site reactions in any group. Animal weights were tracked and presented as group percentage change from initial starting weight. In general, animals gained weight rapidly over the course of the study, with no weight decreases following inoculation regardless of vaccine formulation administered. However, 3 animals were lost over the course of the study due to isofluorane anesthesia given prior to blood collection: 2 animals from group 5 at the 6-hour post inoculation timepoint on Day 0 and 1 animal from group 3 on Day 14.

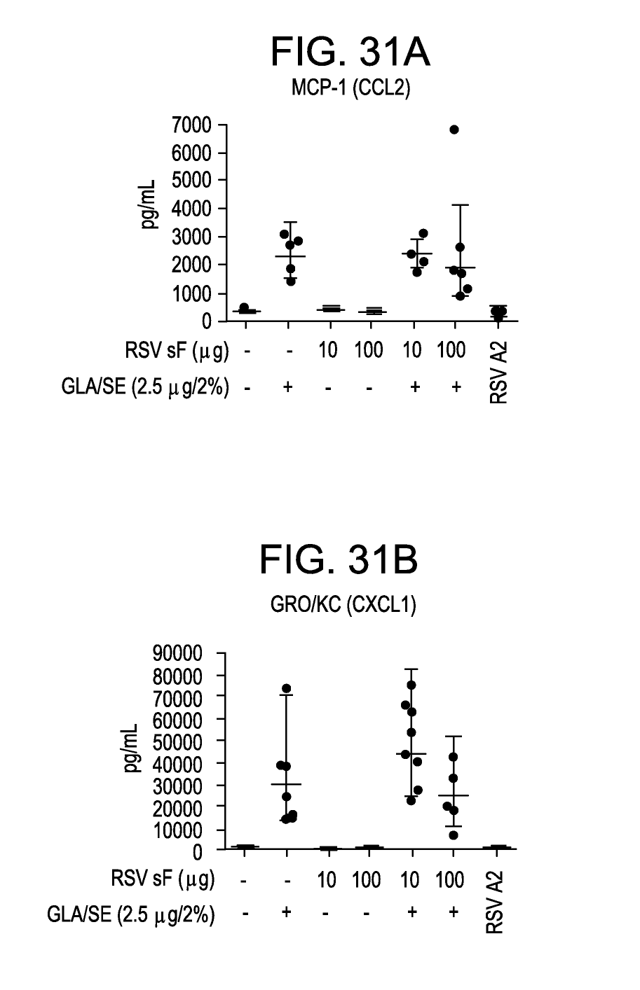

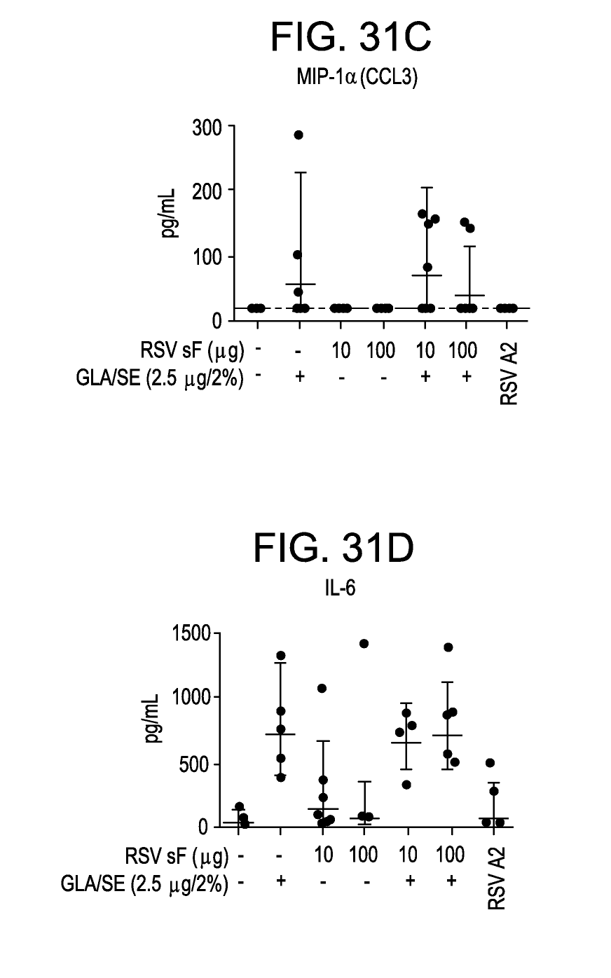

GLA-SE is a TLR4-stimulating adjuvant that has shown activity in mice, guinea pigs, rabbits, monkeys, and humans, but had not previously been evaluated in rats. It has been reported that TLR4 agonist Monophosphoryl Lipid A (MPL)-containing vaccine formulations induce detectable levels of IL-6 and MCP-1 in the serum of mice within the first 6 hours following vaccination (Didierlaurent et al, ASO4, an aluminum salt- and TLR4 agonist-based adjuvant system, induces a transient local immune response leading to enhanced adaptive immunity. J Immunol. 2009; 183:6186-97). These and other serum cytokines were consistently observed in BALB/c mice by 6 hours following GLA-SE administration. To determine whether GLA-SE has innate immune stimulatory activity in the Sprague Dawley rat, serum levels of cytokines including IL-6, MCP-1, MIP-1J3, and KC were evaluated 6 hours post-immunization by a bead-based multiplexed ELISA assay. GLA-SE-dependent serum cytokine responses were observed for each of these cytokines (FIG. 31). The most abundant of these cytokines detected in the serum was KC (CXCL1), a neutrophil chemotactic factor, followed by the monocyte chemotactic factors MCP-1 (CCL2) and MIP-1a (CCL3) and the multipotent cytokine IL-6. While several additional cytokines including IL-1β and TNFα were also investigated, the cytokines shown were the only ones modulated by GLA-SE that were detectable above assay baseline.

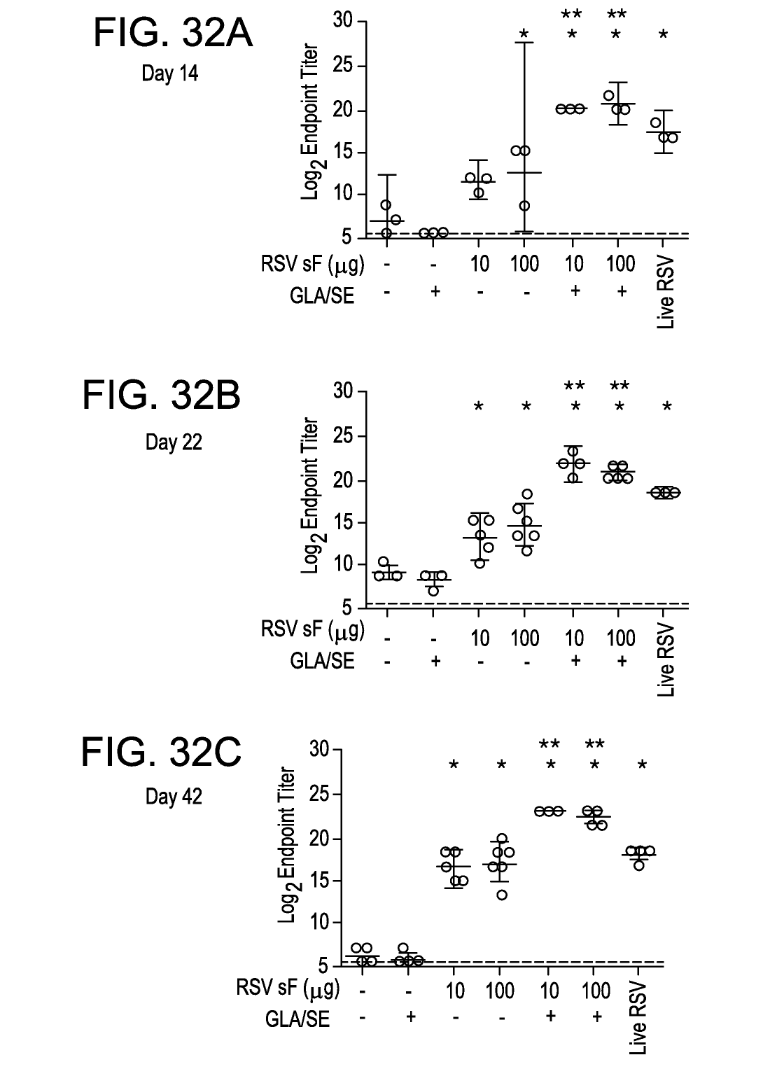

Induced F-directed antibody responses were assessed at Day 14, Day 22, and Day 42 post vaccination and compared to controls for each vaccine cohort (FIG. 32). At each timepoint, the response in the RSV sF+GLA-SE groups was significantly greater than in their matched unadjuvanted RSV sF group. Only the GLA-SE adjuvanted RSV sF cohorts developed serum anti-F IgG endpoint titers greater than that achieved by live RSV, and at Day 42 this difference was significant for both RSV sF+GLA-SE groups. However, at no timepoint was there a significant difference between the IgG titers induced by 10 and 100 g RSV sF, either unadjuvanted or adjuvanted. These results indicate that induction of serum anti-F IgG titers in Sprague Dawley rats was unaffected by increasing the dose of RSV sF from 10 to 100 g but was enhanced by the addition of GLA-SE adjuvant.

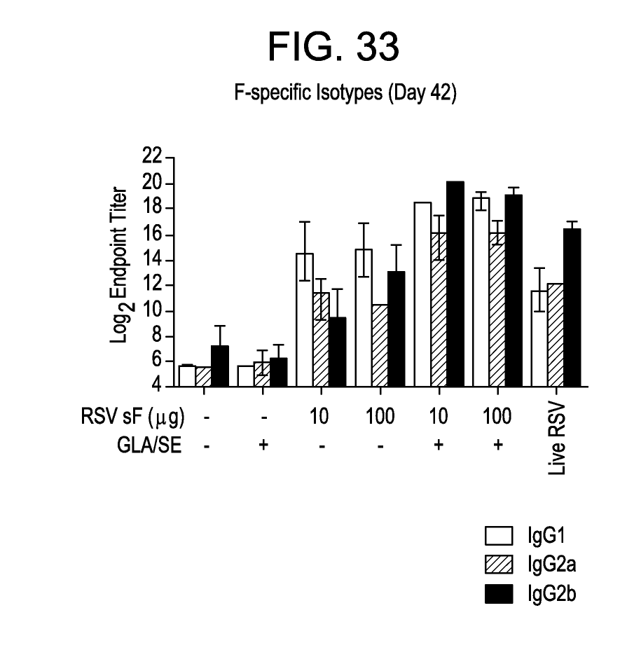

Serum F-specific antibodies at Day 42 were also evaluated for IgG1, IgG2a, and IgG2b isotypes as an indication of the T-helper type balance after vaccination. F-specific IgG1 titers (a Th2-type subtype) and F-specific IgG2a and IgG2b titers (Th1-type subtypes) were both present at Day 42 in rats that received adjuvanted RSV sF vaccines or live RSV A2 (FIG. 33). IgG2a titers were equivalent to IgG1 titers in live RSV groups, suggesting that the Th bias may not be as clearly defined in the rat compared with mice. However, IgG2b titers were higher than IgG1 titers in rats that received live RSV A2, consistent with a Th1-response. Rats that received unadjuvanted RSV sF had higher IgG1 titers than IgG2b titers, consistent with a Th2-response. Rats vaccinated with RSV sF+GLA-SE had higher levels of all isotypes compared to the unadjuvanted RSV sF group at the same dose. Overall, the increase in IgG2b titers (˜64-fold) was greater than the increase in IgG1 titers (˜16-fold) in the groups that were dosed with GLA-SE. This suggests that GLA-SE helps promote a more Th1-biased immune response to RSV sF in Sprague Dawley rats.

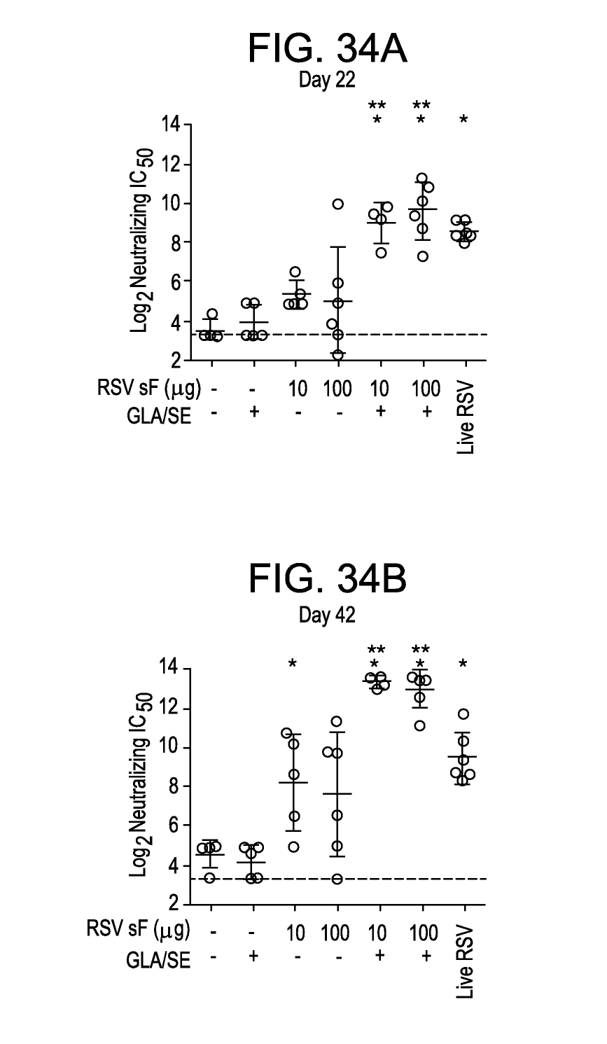

Serum RSV neutralizing titers, a key functional readout for RSV vaccines, were evaluated at Day 22 (22 days post Dose 1) and at Day 42 (20 days post Dose 2). The GMT log₂ IC₅₀ serum neutralizing titers for the different groups of immunized animals at Day 22 ranged from 2.96 in the placebo group to 9.47 in the sF (100 μg)+GLA-SE group (FIG. 34). Rats given unadjuvanted RSV sF vaccines had RSV neutralization titers not significantly different from placebo at Day 22. In contrast, high Day 22 neutralizing titers were achieved by the GLA-SE adjuvanted RSV sF vaccine groups (log₂ GMT 8.89-9.47) and the live RSV group (log₂ GMT 8.51) that were significantly greater than observed in the negative control groups or the paired unadjuvanted RSV sF groups. Neutralizing antibody titers were boosted with a second dose of vaccine as a 10-20 fold enhancement in RSV neutralizing titers in the RSV sF+GLA-SE groups was observed at Day 42 (log₂ GMT 13.25-12.86) compared to Day 22 (log₂ GMT 8.89-9.47). At the Day 42 timepoint as well, RSV sF+GLA-SE immunized groups showed significantly greater neutralizing titers compared to both negative controls and paired unadjuvanted RSV sF groups. The live RSV group also had significantly greater neutralizing titers (log₂ GMT 9.41) compared to negative controls. Interestingly, there was no RSV sF dose-dependence on the vaccine-induced serum neutralizing titers in this study.

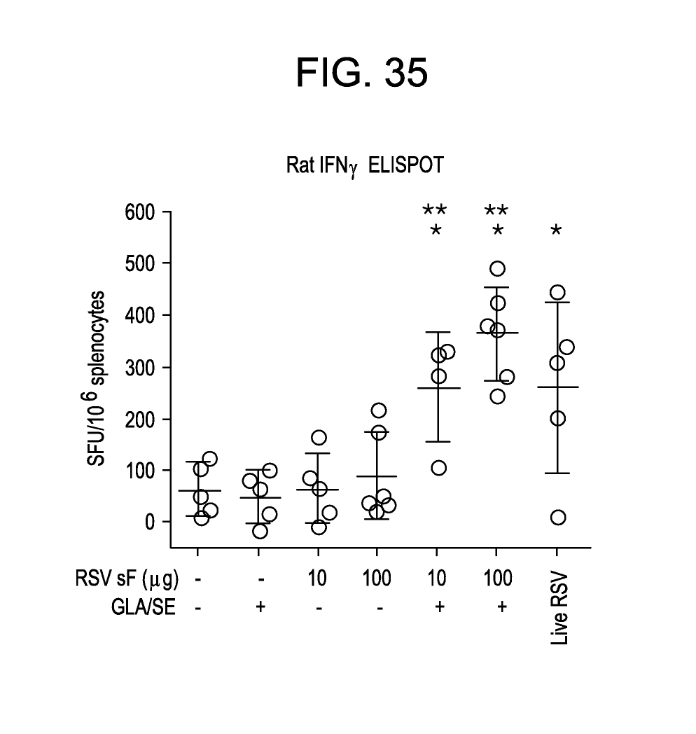

Systemic F-specific T-cell immune responses are another key functional response to RSV-SF vaccination. Splenocytes were harvested from individual animal in each group (n=4-6) at Day 46, 4 days post RSV challenge. Responses were evaluated by IFNγ ELISPOT using RSV sF protein restimulation. The placebo group, adjuvant-alone group, and unadjuvanted RSV sF groups (10 and 100 μg) had equivalent F-specific responses (61.07, 47.73, 64.00, and 87.78 SFU/million cells, respectively). However, both the GLA-SE adjuvanted RSV sF groups (10 and 100 μg) and the live RSV group showed significantly greater F-specific IFNγ ELISPOT responses than the placebo group (259, 362.67, and 258.13 SFU/million cells, respectively) (FIG. 35). This indicated that RSV-SF can prime a T cell response to RSV sF in Sprague Dawley rats in a GLA-SE-dependent manner. While the subtype of T cells (CD4 or CD8) cannot be determined from this assay, exogenous antigens such as the RSV sF protein is most likely restimulating a CD4 response.

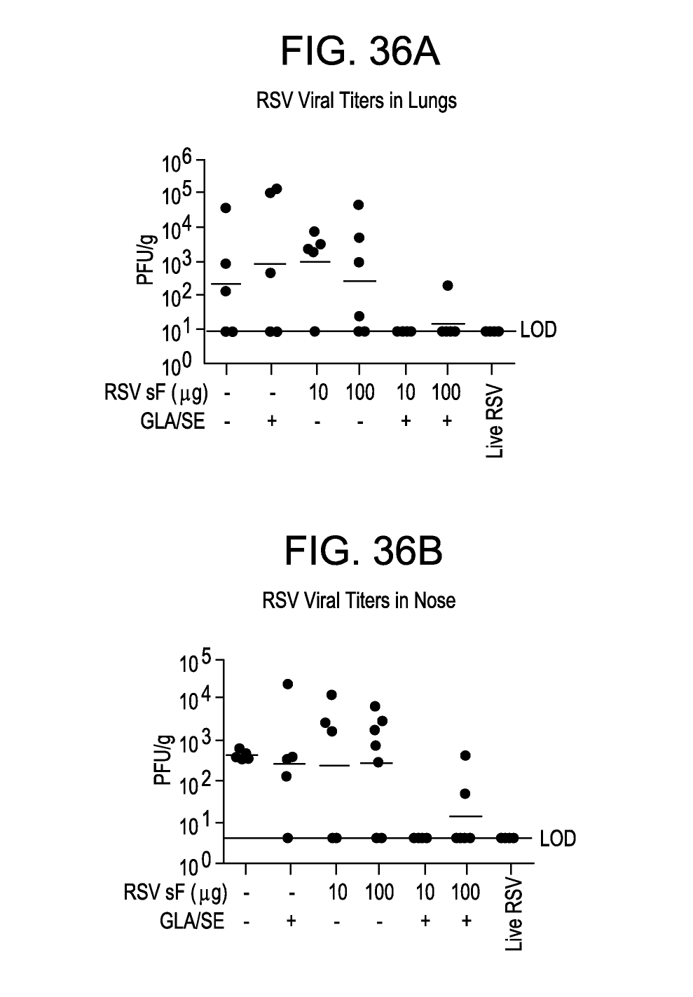

Protection from RSV challenge indicates that the measured immunological responses to vaccination are effective at neutralizing RSV replication in vivo. Following vaccination, all groups were challenged intranasally with 2×10⁶ pfu of RSV A2 virus on Day 42. RSV was titered in homogenized lungs and noses harvested at Day 46 (4 days post challenge). Viral replication in the lung, which was expected in all the negative control animals, was not as consistent in this study as in the initial viral replication timecourse study. In this study, only 3 of 5 placebo animals and 3 of 5 adjuvant-only animals had detectable RSV in the lungs post challenge (FIG. 36). Replication in the nose was more consistent with expected results, with detectable RSV viral loads in 5 of 5 placebo animals and 4 of 5 adjuvant-only animals. The placebo viral titers were 10^{2.30} in the lung (with a 10^{0.94} average LOD) and 10^{2.62} in the nose (with a 100.60 average LOD). Significant RSV protection in non-clinical animal models is historically defined as >10² titer reduction between vaccinated and placebo animals, but this difference was not achieved due to the low levels of replication in the placebo animals. However, prior infection with live RSV A2 fully inhibited RSV replication in the upper and lower respiratory tract of all the challenged animals in this group, with 6 of 6 animals showing no viral titers above the assay LOD in the lung or the nose. In the RSV sF (10 μg)+GLA-SE all 4 animals were also fully protected from RSV challenge in both upper and lower respiratory tract. RSV sF (100 μg)+GLA-SE vaccination inhibited virus replication in the lung in 5 of 6 animals and in the nose of 4 out 6 animals. In contrast, unadjuvanted RSV sF at 10 or 100 μg showed the same spread of viral titers as animals vaccinated with the placebo or GLA-SE alone with titers below the limit of detection in only 1-2 animals per group. This data is consistent with a protective effect of RSV-SF vaccination in Sprague Dawley rats.

Conclusions

This study found that prime-boost inoculations with RSV sF at 10 or 100 μg with 2.5 μg in 2% GLA-SE induces RSV-F-specific humoral and cellular immunity that protected Sprague Dawley rats from RSV challenge. F-specific IgG were detectable as early as Day 14 after a single inoculation with RSV-SF and were characterized as Th1-like (IgG2b>IgG1) by Day 42. Significant titers of RSV neutralizing antibodies were detectable by Day 22 after a single inoculation with RSV-SF and were boosted by a second inoculation with RSV-SF. F-specific T cell responses were detected following challenge in both RSV-SF immunized cohorts. While the high and low dose of RSV sF resulted in comparable humoral and cellular immune responses, the presence of GLA-SE significantly increased the humoral responses and was essential for the cellular response to RSV sF. GLA-SE has innate immune stimulating ability in the rat as demonstrated by the detection of cytokines such as IL-6, KC, MCP-1, and MIP-11a in the serum at 6 hours post inoculation. Innate responses to the vaccine did not result in any weight loss or injection site reactions. While GLA-SE given alone had similar innate immune stimulating ability as RSV sF+GLA-SE, it did not induce RSV specific humoral and cellular responses nor did it protect against RSV challenge. Thus, the Sprague Dawley rat is a suitable toxicology animal model for evaluating the safety of RSV-SF.

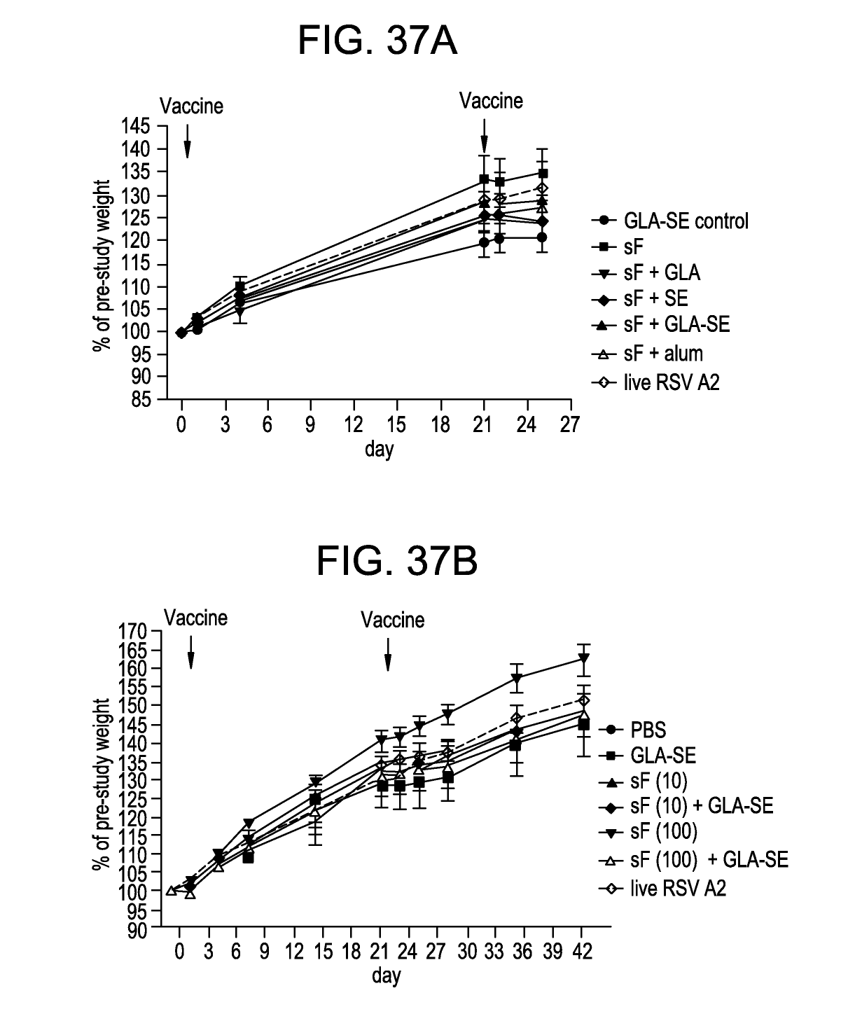

FIGS. 37 A and B are graphs showing injection tolerance for various compositions. (A) weight change in vaccinated cotton rats; and (B) weight change in vaccinated Sprague Dawley (SD) rats. sF+GLA-SE vaccine has acceptable reactogenicity in cotton rats (CR) and Sprague Dawley (SD) rats. No site response, <5% body weight decrease post vaccination.

### Example 4

### Non-Human Primate Immunogenicity Data

### An Adjuvanted RSV sF Vaccine Induces Long-Lasting F-Specific Humoral and Cellular Immunity in Non-Human Primates

Cynomolgus monkeys are a commonly used non-human primate (NHP) species for toxicology and were investigated in terms of their immune responses to an adjuvanted RSV sF candidate vaccine. In this non-GLP study the immunogenicity of an intramuscularly administered RSV vaccine candidate consisting of purified soluble F (sF) protein formulated with a TLR4 agonist glucopyranosyl lipid A (GLA) in a 2% stable emulsion (SE) adjuvant was compared to sF protein alone in cynomolgus monkeys. The first group of 4 NHPs (group 1) was immunized with 100 μg RSV sF without adjuvant while a second group of 4 monkeys (group 2) was immunized with 100 μg RSV sF formulated with 5 μg GLA in 2% SE adjuvant. Animals were immunized at days 0 and 28 and monitored for humoral and cellular responses from Day −7 pre-study through Day 169. The NHPs were then boosted at day 169 with either the unadjuvanted (group 1) or adjuvanted vaccine (group 2) respectively and followed for an additional 14 days (to Day 183) to evaluate long-term memory responses.

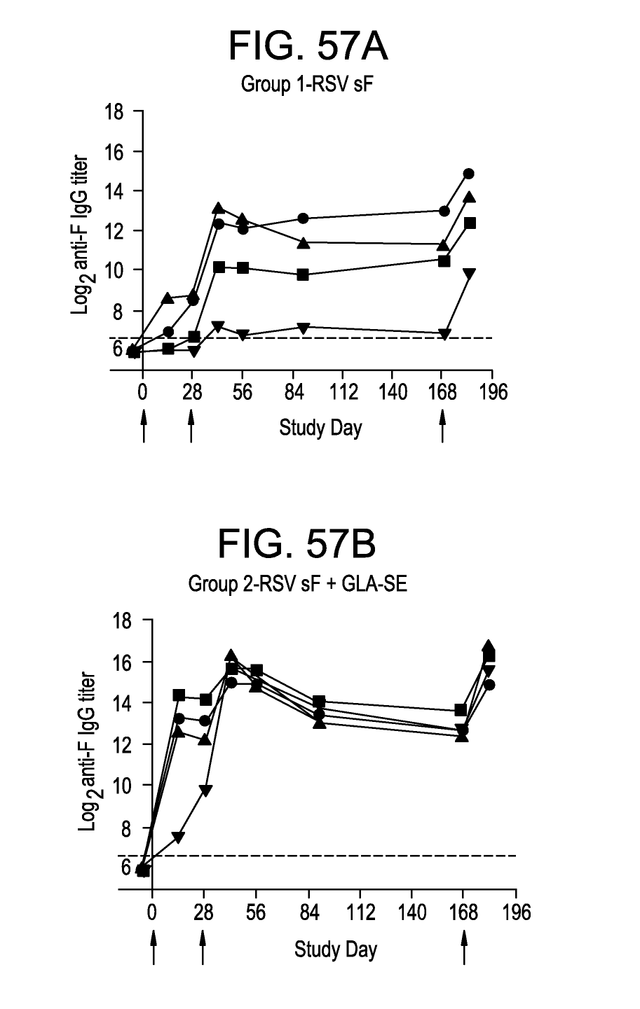

Serological responses were evaluated both in terms of vaccine-induced anti-F IgG titers and in terms of RSV neutralizing antibody (Ab) responses. All the animals in both groups had undetectable anti-F IgG or RSV neutralizing titers prior to immunization, indicating that they were RSV seronegative. Anti-F IgG titers were determined by an RSV sF protein ELISA. At the Day 42 peak of the response, the geomean anti-F IgG titer was significantly higher in group 2 which received RSV sF with GLA-SE (15.67±0.53 log 2) than in group 1 which received RSV sF alone (10.45±2.68 log 2) (p=0.032) (FIG. 57). RSV sF-specific IgG Ab titers dropped over time in both groups (to 12.85 log 2 in Group 2 and 10.13 log 2 in Group 2), but detectable responses were still observed out to Day 169, 5 months post vaccination, at which point the booster vaccination was given. 14 days post recall at Day 183, greater responses were again observed in the sF+GLA-SE group (geomean 15.86±0.85 log 2) compared to the sF alone group (geomean 12.55±2.16 log 2). All the animals in the sF+GLA-SE group demonstrated a ≧4-fold rise in IgG titers at Day 183 compared to Day 169, whereas only 2 of 4 animals in the sF alone group demonstrated a ≧4-fold rise in IgG titers at Day 183 compared to Day 169. These data demonstrate that GLA-SE both enhances the IgG response to sF compared to sF alone and results in a more homogenous response to immunization in the cynomolgus NHP model.

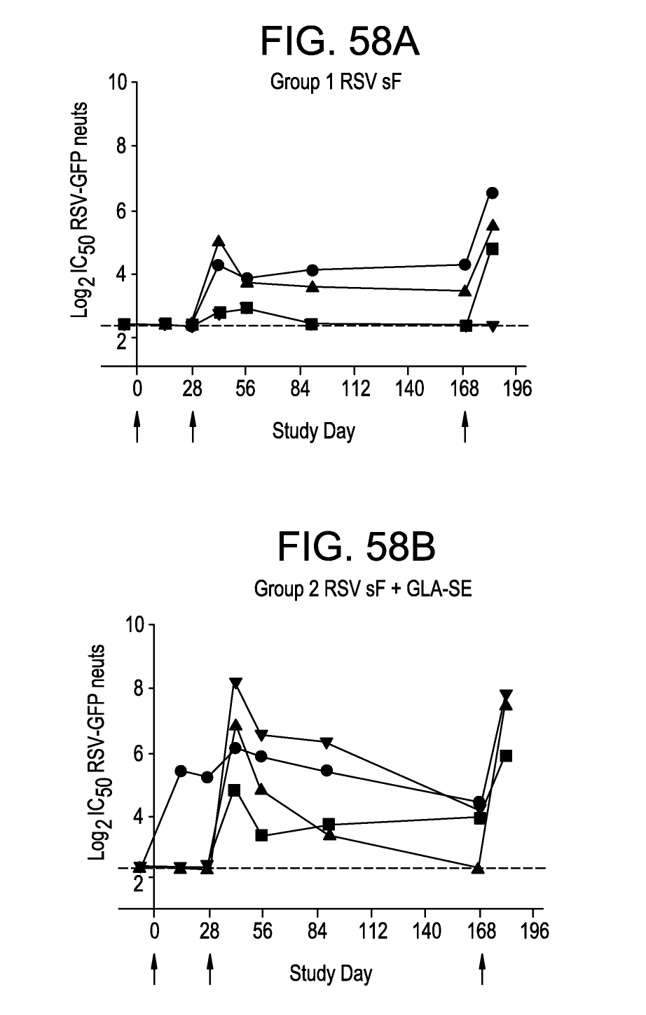

To determine if the addition of GLA-SE to sF also enhanced serum RSV neutralizing titers, RSV neutralizing Ab levels were measured in terms of the log 2 IC50 serum dilution titers necessary to neutralize infection of Vero cells with an RSV A2 strain engineered to express a green fluorescent protein (RSV A2-GFP). At the Day 42 peak of the response, the geometric mean RSV neutralizing Ab titer was significantly higher in the group that received RSV sF with GLA-SE (6.36±1.42 log 2) compared to the group that received RSV sF alone (3.52±1.14 log 2) (p=0.022) (FIG. 58). At the Day 42 peak, 4/4 animals in the RSV sF+GLA-SE group demonstrated a 4-fold boost in neutralizing titers from the Day −7 levels, while only 1/4 animals in the RSV sF alone group demonstrated this 4-fold boost in neutralizing titers. RSV neutralizing Ab titers decreased over time in both groups (to 3.60 log 2 in Group 2 and 2.97 log 2 in Group 1 at Day 169). At Day 169, 5 months post vaccination, the booster vaccination was given. 14 days following the third immunization at Day 183, greater neutralizing Ab titers were observed in the sF+GLA-SE group (geomean 6.70±1.03 log 2) compared to the sF alone group (geomean 4.46±1.79 log 2). These data show that the addition of GLA-SE to sF increases both the magnitude and duration of both the F-specific IgG and RSV neutralizing Ab responses.

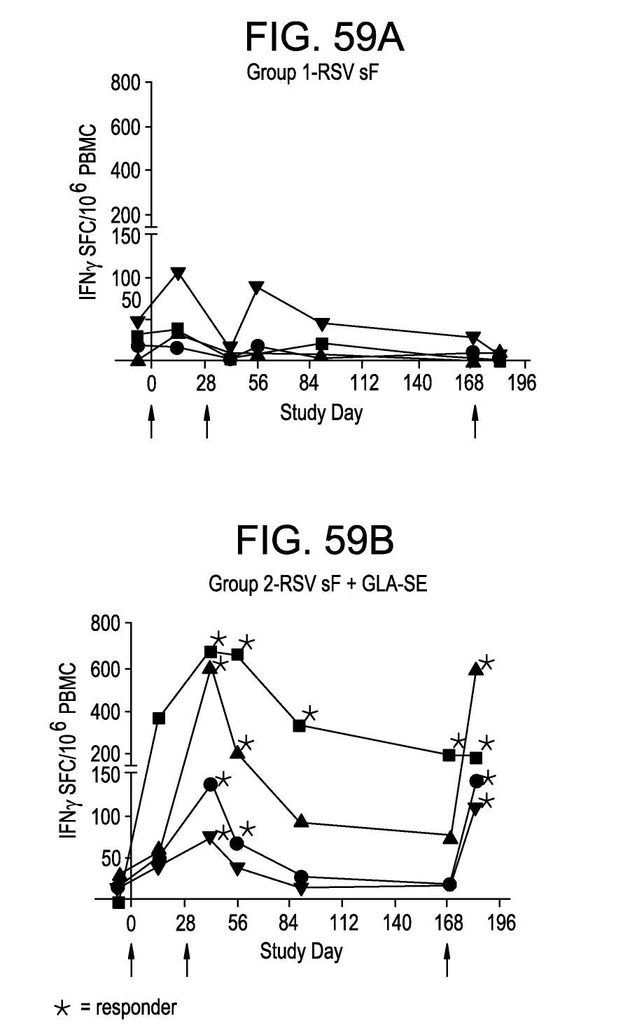

To determine whether immunization with sF formulated with GLA-SE enhanced an F-specific T cell response, F-specific IFNγ T cell responses were measured by ELISPOT following restimulation with a peptide pool of overlapping 15-mers derived from the RSV F protein sequence. At the Day 42 peak of the response, all 4 NHPs in the RSV sF+GLA-SE group showed a positive response, defined as a minimum increase of 50 spot forming counts (SFC)/million PBMC from pre-study baseline (Day −7) and a minimum 4-fold rise in SFC/million PBMC from day −7, while 0 of the 4 monkeys in the RSV sF alone group showed a positive response. At Day 42, the mean response in the sF+GLA-SE group was 392 SFC/million PBMC, significantly greater than that in the F alone group (8 SFC/million PBMC) (p=0.019) (FIG. 59). While the number of T cells in the sF+GLA-SE group decreased over time, one animal still met the definition of a positive responder out to day 169. At Day 169, 5 months post vaccination, a booster vaccination was given. 14 days following the third immunization at Day 183, IFNγ T cells were significantly higher in the 3 monkeys in the sF+GLA-SE group whose responses had waned, to give a total response rate of 4 of 4 animals in the sF+GLA-SE group (mean 261 SFC/million). In comparison, 0 of 4 monkeys in the sF alone group responded with an increase in IFNγ secreting F-specific T cells (mean 5 SFC/million).

In conclusion, robust serum anti-F IgG responses, RSV neutralizing responses, and F-specific IFNγ T cell responses were observed in the sF+GLA-SE immunized animals at levels significantly greater than observed in the unadjuvanted sF alone immunized group. These responses peaked 2 weeks following the second immunization and remained detectable for 3-5 months post vaccination, at which point they were boosted by a third immunization to equivalent or higher levels. These studies indicate that a protein subunit vaccine of RSV sF+GLA-SE can induce robust and long-lived humoral and cellular responses to RSV in non-human primates.

### INCORPORATION BY REFERENCE

All references cited herein, including patents, patent applications, papers, text books and the like, and the references cited therein, to the extent that they are not already, are hereby incorporated herein by reference in their entirety.

### EQUIVALENTS

The foregoing written specification is considered to be sufficient to enable one skilled in the art to practice the invention. The foregoing description and Examples detail certain preferred embodiments of the invention. It will be appreciated, however, that the invention may be practiced in many ways and the invention should be construed in accordance with the appended claims and any equivalents thereof.

1. A vaccine composition comprising: at least about 1 μg and up to about 200 μg RSV soluble F protein and at least about 1 μg and up to about 20 μg of an adjuvant comprising a lipid toll-like receptor (TLR) agonist.

2. The vaccine composition of claim 1, wherein the RSV soluble F protein lacks a C-terminal transmembrane domain.

3. The vaccine composition of claim 2, wherein the RSV soluble F protein lacks a cytoplasmic tail domain.

4. The vaccine composition of claim 3, wherein the RSV soluble F protein comprises amino acids 1-524 of RSV soluble F protein from human strain A2 (SEQ ID NO: 2).

5. The vaccine composition of claim 4, wherein the RSV soluble F protein comprises SEQ ID NO. 7.

6. The vaccine composition of claim 1, wherein the adjuvant comprises a (TLR)4 agonist.

7. The vaccine composition of claim 6, wherein the adjuvant comprises a synthetic hexylated Lipid A derivative.

8. The vaccine composition of claim 7, wherein the adjuvant comprises Glucopyraonsyl Lipid A (GLA).

9. The vaccine composition of claim 8, wherein the adjuvant comprises a compound having a formula: 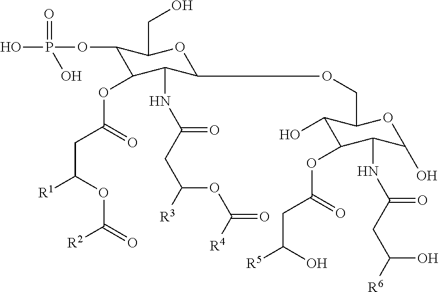
wherein R¹, R³, R⁵ and R⁶, are C₁₁-C₂₀ alkyl; and R² and R⁴ are C₁₂-C₂₀ alkyl.

10. The vaccine composition of claim 9, wherein the adjuvant comprises GLA in a stable oil-in-water emulsion (GLA-SE).

11. The vaccine composition of claim 10, wherein the adjuvant comprises GLA in a stabilized squalene based emulsion.

12. The vaccine composition of claim 11, wherein the adjuvant comprises GLA in a stabilized oil-in-water emulsion having a concentration of at least about 1% and up to about 5%.

13. The vaccine composition of claim 12, wherein the adjuvant comprises GLA in a stabilized oil-in-water emulsion having a mean particle size of at least about 50 nm and up to about 200 nm.

14. The vaccine composition of claim 1, comprising at least about 5 μg, at least about 10 μg, at least about 20 μg, at least about 30 μg, at least about 50 μg, or at least about 100 μg RSV soluble F protein.

15.-19. (canceled)

20. The vaccine composition of claim 6, comprising at least about 2.5 g or at least about 5 g adjuvant.

21. (canceled)

22. The vaccine composition of claim 6, comprising between about 10 g and about 100 g RSV soluble F protein and between about 1 g and about 5 g GLA-SE.

23. The vaccine composition of claim 1, comprising between about 10 g and about 100 g RSV soluble F protein, wherein RSV soluble F protein comprises amino acids 1-524 of RSV soluble F protein from human strain A2 (SEQ ID NO: 2) and between about 1 g and about 5 g GLA in a stabilized oil-in-water emulsion having a concentration between about 1% and 5%.

24.-27. (canceled)

28. The vaccine composition of claim 23, comprising a volume of between about 50 μl and about 500 μl.

29. A method of preventing respiratory syncytial virus (RSV) infection in a mammal, the method comprising: administering to the mammal a therapeutically effective amount of a vaccine composition comprising: at least about 1 g and up to about 200 g RSV soluble F protein at a concentration of and at least about 1 g and up to about 20 g of an adjuvant comprising a lipid toll-like receptor (TLR) agonist, sufficient to prevent RSV infection in the mammal.

30.-83. (canceled)


SEQUENCE LISTING (13 sequences)

SEQ ID NO: 1 - DNA, 1725 bp, Human respiratory syncytial virus
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagtcagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgccggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 2 - protein, 1621 aa, Human respiratory syncytial virus
METGLLELEILELELYSALAASNALAILETHRTHRILELETHRMETGLLELEILELELYSALAASNALAILETHRTHRILELETHRTYRGLNSERTHRCYSSERALAVALSERLYSGLYTYRLESERALALEARGTHRGLYTRPTYRTHRSERVALILETHRILEGLLESERASNILELYSLYSASNLYSCYSASNGLYTHRASPALALYSVALLYSLEILELYSGLNGLLEASPLYSTYRLYSASNALAVALTHRGLLEGLNLELEMETGLNSERTHRPRALATHRASNASNARGALAARGARGGLLEPRARGPHEMETASNTYRTHRLEASNASNALALYSLYSTHRASNVALTHRLESERLYSLYSARGLYSARGARGPHELEGLYPHELELEGLYVALGLYSERALAILEALASERGLYVALALAVALSERLYSVALLEHISLEGLGLYGLVALASNLYSILELYSSERALALELESERTHRASNLYSALAVALVALSERLESERASNGLYVALSERVALLETHRSERLYSVALLEASPLELYSASNTYRILEASPLYSGLNLELEPRILEVALASNLYSGLNSERCYSSERILESERASNILEGLTHRVALILEGLPHEGLNGLNLYSASNASNARGLELEGLILETHRARGGLPHESERVALASNALAGLYVALTHRTHRPRVALSERTHRTYRMETLETHRASNSERGLLELESERLEILEASNASPMETPRILETHRASNASPGLNLYSLYSLEMETSERASNASNVALGLNILEVALARGGLNGLNSERTYRSERILEMETSERILEILELYSGLGLVALLEALATYRVALVALGLNLEPRLETYRGLYVALILEASPTHRPRCYSTRPLYSLEHISTHRSERPRLECYSTHRTHRASNTHRLYSGLGLYSERASNILECYSLETHRARGTHRASPARGGLYTRPTYRCYSASPASNALAGLYSERVALSERPHEPHEPRGLNALAGLTHRCYSLYSVALGLNSERASNARGVALPHECYSASPTHRMETASNSERLETHRLEPRSERGLVALASNLECYSASNVALASPILEPHEASNPRLYSTYRASPCYSLYSILEMETTHRSERLYSTHRASPVALSERSERSERVALILETHRSERLEGLYALAILEVALSERCYSTYRGLYLYSTHRLYSCYSTHRALASERASNLYSASNARGGLYILEILELYSTHRPHESERASNGLYCYSASPTYRVALSERASNLYSGLYVALASPTHRVALSERVALGLYASNTHRLETYRTYRVALASNLYSGLNGLGLYLYSSERLETYRVALLYSGLYGLPRILEILEASNPHETYRASPPRLEVALPHEPRSERASPGLPHEASPALASERILESERGLNVALASNGLLYSILEASNGLNSERLEALAPHEILEARGLYSSERASPGLLELEHISASNVALASNALAGLYLYSSERTHRTHRASNILEMETILETHRTHRILEILEILEVALILEILEVALILELELESERLEILEALAVALGLYLELELETYRCYSLYSALAARGSERTHRPRVALTHRLESERLYSASPGLNLESERGLYILEASNASNILEALAPHESERASN

SEQ ID NO: 3 - DNA, 1575 bp, Artificial Sequence
  Synthetic polynucleotide
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacaccagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagtcagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgccggtaaatccaccacaaattaa

SEQ ID NO: 4 - DNA, 1575 bp, Artificial Sequence
  Synthetic polynucleotide
atggaacttcttattctcaaagccaatgcgattacaacaatccttactgctgtaaccttctgcttcgcatctggacagaatatcaccgaggaattctatcaatccacctgcagcgcggtgtcaaaggggtatctttccgcattgagaacaggttggtatacatccgttattactattgagctgtctaacatcaagaagaataaatgtaatggaactgacgcaaaagtgaagctgatcaagcaggagcttgataagtacaaaaacgctgtgacagaactccagctcctcatgcagagcaccccggcgacgaacaatagagcgcggcgcgagctgcctaggtttatgaattatacccttaacaacgctaagaagacaaacgtgacgctctcaaagaagaggaaacgaaggtttcttggattcctgctcggggtgggatccgctattgcaagcggcgtggcggtttcaaaggtcctccacctggagggggaagtgaacaagattaagtcagcactcctgagtacaaacaaagcagtggtttctctgagcaacggagtgtcagtattgacgagcaaggtgcttgacctcaagaactacattgacaaacagctgctgcccatagtgaacaaacagtcatgctccatctccaatatcgagacagtcatcgaattccagcagaagaacaacagactcctggaaatcacacgggagtttagcgtgaatgcgggcgtaacaactcccgtgtccacctacatgctgacaaattctgagctgctgagtctgataaatgatatgcctattacaaatgaccagaagaagttgatgtccaacaatgtgcaaatagtcagacagcagtcttatagtattatgagcatcatcaaagaggaagttcttgcctatgttgtacaactgcccctctacggggtcatcgacacaccctgttggaagctgcacacctcacctctgtgcaccaccaacacgaaagagggtagcaacatctgtctgactaggactgacaggggttggtactgcgataacgccggtagcgtgtcatttttcccacaagcagagacttgtaaagtacagtccaacagggtcttttgtgacacaatgaattctcttaccctgcccagcgaagttaatctgtgtaacgtcgatatctttaatccaaagtacgattgtaaaatcatgacatctaaaaccgatgtgagcagcagcgttattacaagtcttggcgctatcgtcagctgttacggaaaaaccaagtgcacggcatccaacaagaatagaggcattataaagaccttcagtaatgggtgtgactacgttagcaataagggcgtagacaccgtctccgtaggaaacacactgtactatgtaaataaacaagaaggcaaatccctttatgtgaagggggagcctatcattaatttctacgaccctctggttttcccgagtgacgagttcgatgccagcatatcccaagtgaatgagaaaatcaaccagtccttggcctttataaggaaaagcgatgagcttctgcacaacgtgaatgccggtaaatccaccacaaactag

SEQ ID NO: 5 - DNA, 1575 bp, Artificial Sequence
  Synthetic polynucleotide
atggagttgctcatcctcaaggccaacgccatcaccacgatcctcacggccgtcacgttctgcttcgcgtccggccagaacatcaccgaggagttctaccagtcgacgtgcagcgccgtgagcaagggctacctcagcgcgctgaggacgggctggtacaccagcgtcatcacgatcgagttgagcaacatcaagaagaacaagtgcaacggcaccgacgcgaaggtcaagttgatcaagcaggagttggacaagtacaagaacgccgtgaccgagttgcagttgctcatgcagagcacgccggcgacgaacaaccgcgccaggagggagctcccgaggttcatgaactacacgctcaacaacgccaagaagaccaacgtgaccttgagcaagaagaggaagaggaggttcctcggcttcttgttgggcgtcggctcggccatcgccagcggcgtggccgtctcgaaggtcctgcacctggagggcgaggtgaacaagatcaagagcgcgctgctctccacgaacaaggccgtcgtcagcttgtccaacggcgtcagcgtcttgaccagcaaggtgttggacctcaagaactacatcgacaagcagttgttgccgatcgtgaacaagcagagctgcagcatctcgaacatcgagaccgtgatcgagttccagcagaagaacaacaggctgctcgagatcaccagggagttcagcgtcaacgccggcgtcacgacgccggtcagcacctacatgttgaccaacagcgagttgttgtccttgatcaacgacatgccgatcaccaacgaccagaagaagttgatgtccaacaacgtgcagatcgtcaggcagcagagctactcgatcatgtccatcatcaaggaggaggtcttggcctacgtcgtgcagttgccgctgtacggcgtcatcgacacgccctgctggaagctgcacacgtccccgctgtgcacgaccaacacgaaggaggggtccaacatctgcttgaccaggaccgacaggggctggtactgcgacaacgccggctccgtgtcgttcttcccgcaggccgagacctgcaaggtccagtccaaccgcgtcttctgcgacacgatgaacagcttgacgttgccgagcgaggtcaacctctgcaacgtcgacatcttcaaccccaagtacgactgcaagatcatgacgtccaagaccgacgtcagcagctccgtgatcacgtcgctcggcgccatcgtgtcctgctacggcaagaccaagtgcaccgcgtccaacaagaaccgcggcatcatcaagacgttctcgaacgggtgcgactacgtctcgaacaagggggtggacaccgtgtccgtcggcaacacgttgtactacgtcaacaagcaggagggcaagagcctctacgtcaagggcgagccgatcatcaacttctacgacccgttggtcttcccctcggacgagttcgacgcgtcgatctcgcaggtcaacgagaagatcaaccagagcctggcgttcatccggaagtccgacgagttgttgcacaacgtgaacgccggcaagtccaccacgaactaa

SEQ ID NO: 6 - DNA, 3150 bp, Artificial Sequence
  Synthetic polynucleotide
atggagttgctcatcctcaaggccaacgccatcaccacgatcctcacggcagtcacattctgtttcgcttctggtcagaacatcactgaggaattctaccaatcgacgtgcagtgcagttagcaagggctatctcagtgctctgagaacgggttggtataccagtgtcatcactatcgagttgagtaacatcaagaagaacaagtgtaacggaaccgatgcgaaggtaaagttgatcaagcaggagttggacaagtacaagaacgctgtaacagagttgcagttgctcatgcagagcacaccagcgacgaacaaccgagccaggagagagctaccaaggttcatgaactacacgctcaacaacgccaagaagaccaacgtgacattgagcaagaagaggaagaggagattcctcggtttcttgttgggtgtcggatctgcaatcgccagtggcgttgctgtctcgaaggtcctgcacctagaaggggaagtgaacaagatcaagagtgctctgctatccacgaacaaggctgtcgtcagcttgtcaaacggagtcagtgtcttgaccagcaaggtgttggacctcaagaactacatcgacaagcagttgttacctatcgtgaacaagcaaagctgcagcatctcaaacatcgagactgtgatcgagttccagcagaagaacaacagactactagagatcaccagggagttcagtgtcaacgcaggtgtaacgacacctgtcagcacttacatgttgactaacagtgagttgttgtcattgatcaacgacatgcctatcaccaacgatcagaagaagttgatgtccaacaacgtgcagatcgtcagacagcagagctactcgatcatgtccatcatcaaggaggaagtcttggcatacgtagtacagttgccactgtatggtgtcatcgacacaccctgctggaagctgcacacgtcccctctatgtacgaccaacacgaaggaagggtccaacatctgcttgaccaggactgacagaggatggtactgcgacaacgcaggatccgtgtcgttcttcccacaggctgagacctgcaaggtccagtccaaccgagtcttctgcgacacgatgaacagcttgacgttgccgagtgaggtaaacctctgcaacgtcgacatcttcaaccccaagtacgactgcaagatcatgacgtccaagaccgatgtcagcagctccgtgatcacatcgctcggagccatcgtgtcatgctacggcaagaccaagtgcacagcgtccaacaagaaccgtggaatcatcaagacgttctcgaacgggtgcgactacgtctcaaacaagggggtggacactgtgtctgtaggcaacacattgtactacgtaaacaagcaggaaggtaagagcctctacgtcaagggtgaaccaatcatcaacttctacgacccgttggtcttcccctctgacgagttcgacgcatcgatctctcaggtcaacgagaagatcaaccagagcctagcattcatccggaagtccgacgagttgttgcacaacgtgaatgccggtaagtccaccacaaactaaatggagttgctcatcctcaaggccaacgccatcaccacgatcctcacggcagtcacattctgtttcgcttctggtcagaacatcactgaggaattctaccaatcgacgtgcagtgcagttagcaagggctatctcagtgctctgagaacgggttggtataccagtgtcatcactatcgagttgagtaacatcaagaagaacaagtgtaacggaaccgatgcgaaggtaaagttgatcaagcaggagttggacaagtacaagaacgctgtaacagagttgcagttgctcatgcagagcacaccagcgacgaacaaccgagccaggagagagctaccaaggttcatgaactacacgctcaacaacgccaagaagaccaacgtgacattgagcaagaagaggaagaggagattcctcggtttcttgttgggtgtcggatctgcaatcgccagtggcgttgctgtctcgaaggtcctgcacctagaaggggaagtgaacaagatcaagagtgctctgctatccacgaacaaggctgtcgtcagcttgtcaaacggagtcagtgtcttgaccagcaaggtgttggacctcaagaactacatcgacaagcagttgttacctatcgtgaacaagcaaagctgcagcatctcaaacatcgagactgtgatcgagttccagcagaagaacaacagactactagagatcaccagggagttcagtgtcaacgcaggtgtaacgacacctgtcagcacttacatgttgactaacagtgagttgttgtcattgatcaacgacatgcctatcaccaacgatcagaagaagttgatgtccaacaacgtgcagatcgtcagacagcagagctactcgatcatgtccatcatcaaggaggaagtcttggcatacgtagtacagttgccactgtatggtgtcatcgacacaccctgctggaagctgcacacgtcccctctatgtacgaccaacacgaaggaagggtccaacatctgcttgaccaggactgacagaggatggtactgcgacaacgcaggatccgtgtcgttcttcccacaggctgagacctgcaaggtccagtccaaccgagtcttctgcgacacgatgaacagcttgacgttgccgagtgaggtaaacctctgcaacgtcgacatcttcaaccccaagtacgactgcaagatcatgacgtccaagaccgatgtcagcagctccgtgatcacatcgctcggagccatcgtgtcatgctacggcaagaccaagtgcacagcgtccaacaagaaccgtggaatcatcaagacgttctcgaacgggtgcgactacgtctcaaacaagggggtggacactgtgtctgtaggcaacacattgtactacgtaaacaagcaggaaggtaagagcctctacgtcaagggtgaaccaatcatcaacttctacgacccgttggtcttcccctctgacgagttcgacgcatcgatctctcaggtcaacgagaagatcaaccagagcctagcattcatccggaagtccgacgagttgttgcacaacgtgaatgccggtaagtccaccacaaactaa

SEQ ID NO: 7 - protein, 1483 aa, Artificial Sequence
  Synthetic polypeptide
METGLLELEILELELYSALAASNALAILETHRTHRILELETHRALAVALTHRPHECYSPHEALASERGLYGLNASNILETHRGLGLPHETYRGLNSERTHRCYSSERALAVALSERLYSGLYTYRLESERALALEARGTHRGLYTRPTYRTHRSERVALILETHRILEGLLESERASNILELYSLYSASNLYSCYSASNGLYTHRASPALALYSVALLYSLEILELYSGLNGLLEASPLYSTYRLYSASNALAVALTHRGLLEGLNLELEMETGLNSERTHRPRALATHRASNASNARGALAARGARGGLLEPRARGPHEMETASNTYRTHRLEASNASNALALYSLYSTHRASNVALTHRLESERLYSLYSARGLYSARGARGPHELEGLYPHELELEGLYVALGLYSERALAILEALASERGLYVALALAVALSERLYSVALLEHISLEGLGLYGLVALASNLYSILELYSSERALALELESERTHRASNLYSALAVALVALSERLESERASNGLYVALSERVALLETHRSERLYSVALLEASPLELYSASNTYRILEASPLYSGLNLELEPRILEVALASNLYSGLNSERCYSSERILESERASNILEGLTHRVALILEGLPHEGLNGLNLYSASNASNARGLELEGLILETHRARGGLPHESERVALASNALAGLYVALTHRTHRPRVALSERTHRTYRMETLETHRASNSERGLLELESERLEILEASNASPMETPRILETHRASNASPGLNLYSLYSLEMETSERASNASNVALGLNILEVALARGGLNGLNSERTYRSERILEMETSERILEILELYSGLGLVALLEALATYRVALVALGLNLEPRLETYRGLYVALILEASPTHRPRCYSTRPLYSLEHISTHRSERPRLECYSTHRTHRASNTHRLYSGLGLYSERASNILECYSLETHRARGTHRASPARGGLYTRPTYRCYSASPASNALAGLYSERVALSERPHEPHEPRGLNALAGLTHRCYSLYSVALGLNSERASNARGVALPHECYSASPTHRMETASNSERLETHRLEPRSERGLVALASNLECYSASNVALASPILEPHEASNPRLYSTYRASPCYSLYSILEMETTHRSERLYSTHRASPVALSERSERSERVALILETHRSERLEGLYALAILEVALSERCYSTYRGLYLYSTHRLYSCYSTHRALASERASNLYSASNARGGLYILEILELYSTHRPHESERASNGLYCYSASPTYRVALSERASNLYSGLYVALASPTHRVALSERVALGLYASNTHRLETYRTYRVALASNLYSGLNGLGLYLYSSERLETYRVALLYSGLYGLPRILEILEASNPHETYRASPPRLEVALPHEPRSERASPGLPHEASPALASERILESERGLNVALASNGLLYSILEASNGLNSERLEALAPHEILEARGLYSSERASPGLLELEHISASNVALASNALAGLYLYSSERTHRTHRASN

SEQ ID NO: 8 - DNA, 1725 bp, Artificial Sequence
  Synthetic polynucleotdie
atggaacttcttattctcaaagccaatgcgattacaacaatccttactgctgtaaccttctgcttcgcatctggacagaatatcaccgaggaattctatcaatccacctgcagcgcggtgtcaaaggggtatctttccgcattgagaacaggttggtatacatccgttattactattgagctgtctaacatcaagaagaataaatgtaatggaactgacgcaaaagtgaagctgatcaagcaggagcttgataagtacaaaaacgctgtgacagaactccagctcctcatgcagagcaccccggcgacgaacaatagagcgcggcgcgagctgcctaggtttatgaattatacccttaacaacgctaagaagacaaacgtgacgctctcaaagaagaggaaacgaaggtttcttggattcctgctcggggtgggatccgctattgcaagcggcgtggcggtttcaaaggtcctccacctggagggggaagtgaacaagattaagtcagcactcctgagtacaaacaaagcagtggtttctctgagcaacggagtgtcagtattgacgagcaaggtgcttgacctcaagaactacattgacaaacagctgctgcccatagtgaacaaacagtcatgctccatctccaatatcgagacagtcatcgaattccagcagaagaacaacagactcctggaaatcacacgggagtttagcgtgaatgcgggcgtaacaactcccgtgtccacctacatgctgacaaattctgagctgctgagtctgataaatgatatgcctattacaaatgaccagaagaagttgatgtccaacaatgtgcaaatagtcagacagcagtcttatagtattatgagcatcatcaaagaggaagttcttgcctatgttgtacaactgcccctctacggggtcatcgacacaccctgttggaagctgcacacctcacctctgtgcaccaccaacacgaaagagggtagcaacatctgtctgactaggactgacaggggttggtactgcgataacgccggtagcgtgtcatttttcccacaagcagagacttgtaaagtacagtccaacagggtcttttgtgacacaatgaattctcttaccctgcccagcgaagttaatctgtgtaacgtcgatatctttaatccaaagtacgattgtaaaatcatgacatctaaaaccgatgtgagcagcagcgttattacaagtcttggcgctatcgtcagctgttacggaaaaaccaagtgcacggcatccaacaagaatagaggcattataaagaccttcagtaatgggtgtgactacgttagcaataagggcgtagacaccgtctccgtaggaaacacactgtactatgtaaataaacaagaaggcaaatccctttatgtgaagggggagcctatcattaatttctacgaccctctggttttcccgagtgacgagttcgatgccagcatatcccaagtgaatgagaaaatcaaccagtccttggcctttataaggaaaagcgatgagcttctgcacaacgtgaatgccggtaaatccaccacaaacataatgatcaccactatcattatcgtcattattgtgatcttgctgagcctcatcgctgtggggctcctcttgtattgcaaagcccgctcaaccccagtcactctctctaaagaccaactgtctgggatcaataacatagccttttcaaattag

SEQ ID NO: 9 - DNA, 1725 bp, Artificial Sequence
  Synthetic polynucleotide
atggagttgctcatcctcaaggccaacgccatcaccacgatcctcacggccgtcacgttctgcttcgcgtccggccagaacatcaccgaggagttctaccagtcgacgtgcagcgccgtgagcaagggctacctcagcgcgctgaggacgggctggtacaccagcgtcatcacgatcgagttgagcaacatcaagaagaacaagtgcaacggcaccgacgcgaaggtcaagttgatcaagcaggagttggacaagtacaagaacgccgtgaccgagttgcagttgctcatgcagagcacgccggcgacgaacaaccgcgccaggagggagctcccgaggttcatgaactacacgctcaacaacgccaagaagaccaacgtgaccttgagcaagaagaggaagaggaggttcctcggcttcttgttgggcgtcggctcggccatcgccagcggcgtggccgtctcgaaggtcctgcacctggagggcgaggtgaacaagatcaagagcgcgctgctctccacgaacaaggccgtcgtcagcttgtccaacggcgtcagcgtcttgaccagcaaggtgttggacctcaagaactacatcgacaagcagttgttgccgatcgtgaacaagcagagctgcagcatctcgaacatcgagaccgtgatcgagttccagcagaagaacaacaggctgctcgagatcaccagggagttcagcgtcaacgccggcgtcacgacgccggtcagcacctacatgttgaccaacagcgagttgttgtccttgatcaacgacatgccgatcaccaacgaccagaagaagttgatgtccaacaacgtgcagatcgtcaggcagcagagctactcgatcatgtccatcatcaaggaggaggtcttggcctacgtcgtgcagttgccgctgtacggcgtcatcgacacgccctgctggaagctgcacacgtccccgctgtgcacgaccaacacgaaggaggggtccaacatctgcttgaccaggaccgacaggggctggtactgcgacaacgccggctccgtgtcgttcttcccgcaggccgagacctgcaaggtccagtccaaccgcgtcttctgcgacacgatgaacagcttgacgttgccgagcgaggtcaacctctgcaacgtcgacatcttcaaccccaagtacgactgcaagatcatgacgtccaagaccgacgtcagcagctccgtgatcacgtcgctcggcgccatcgtgtcctgctacggcaagaccaagtgcaccgcgtccaacaagaaccgcggcatcatcaagacgttctcgaacgggtgcgactacgtctcgaacaagggggtggacaccgtgtccgtcggcaacacgttgtactacgtcaacaagcaggagggcaagagcctctacgtcaagggcgagccgatcatcaacttctacgacccgttggtcttcccctcggacgagttcgacgcgtcgatctcgcaggtcaacgagaagatcaaccagagcctggcgttcatccggaagtccgacgagttgttgcacaacgtgaacgccggcaagtccaccacgaacatcatgatcacgacgatcatcatcgtgatcatcgtgatcttgttgtcgttgatcgccgtcggcctgctcttgtactgcaaggccaggagcacgcccgtcacgctgagcaaggaccagctgagcggcatcaacaacatcgcgttcagcaactaa

SEQ ID NO: 10 - protein, 16 aa, Artificial Sequence
  Synthetic peptide
GWYTSVITIELSNIKE

SEQ ID NO: 11 - protein, 15 aa, Artificial Sequence
  Synthetic peptide
VSVLTSKVLDLKNYI

SEQ ID NO: 12 - protein, 9 aa, Artificial Sequence
  Synthetic peptide
KYKNAVTEL

SEQ ID NO: 13 - protein, 9 aa, Artificial Sequence
  Synthetic peptide
SYIGSINNI